(19) United States
(12) Patent Application Publication
(10) Pub. No.: US 2012/0129875 A1
(43) Pub. Date: May 24, 2012

(54) SUBSTITUTED QUINAZOLINES AS FUNGICIDES

(75) Inventors: Laura Quaranta, Stein (CH); Clemens Lamberth, Stein (CH); David Guillaume Claude François Lefranc, Vancouver (CA); Jayant Umarye, Ilhas (IN); Peter Renold, Stein (CH); Andrew Edmunds, Stein (CH); Martin Pouliot, Stein (CH)

(73) Assignee: SYNGENTA CROP PROTECTION, LLC, Greensboro, NC (US)

(21) Appl. No.: 13/322,949

(22) PCT Filed: May 26, 2010
(86) PCT No.: PCT/EP2010/057220

§ 371 Date: Feb. 13, 2012

(30) Foreign Application Priority Data
May 29, 2009 (IN) 1102/DEL/2009

(51) Int. Cl. A01N 43/54; C07D 401/14; A01P 3/00; C07D 401/04

(52) U.S. Cl. 514/266.21; 544/284

(57) ABSTRACT

The present invention relates to a compound of formula (I) wherein the substituents have the definitions as defined in claim 1 or a salt or a N-oxide thereof, their use and methods for the control and/or prevention of microbial infection, particularly fungal infection, in plants and to processes for the preparation of these compounds.

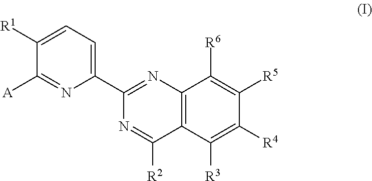

The present invention relates to novel quinazoline containing compounds, their use in compositions and methods for the control and/or prevention of microbial infection, particularly fungal infection, in plants and to processes for the preparation of these compounds.

The incidence of serious microbial infections, particularly fungal infections, either systemic or topical, continues to increase for plants.

Fungicides are compounds, of natural or synthetic origin, which act to protect plants against damage caused by fungi. Current methods of agriculture rely heavily on the use of fungicides. In fact, some crops cannot be grown usefully without the use of fungicides. Using fungicides allows a grower to increase the yield of the crop and consequently, increase the value of the crop. Numerous fungicidal agents have been developed. However, the treatment of fungal infestations continues to be a major problem. Furthermore, fungicide resistance has become a serious problem, rendering these agents ineffective for some agricultural uses. As such, a need exists for the development of new fungicidal compounds.

Accordingly, the present invention provides a compound of formula I:

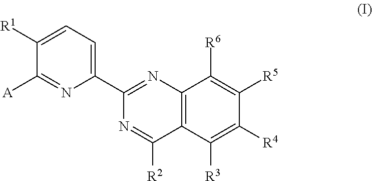

wherein:
R¹ is hydrogen, hydroxyl, halo, cyano, C₁₋₈ alkyl, C₁₋₈ haloalkyl, C₁₋₈ alkoxy, C₁₋₈ haloalkoxy, C₁₋₈ alkylthio or C₃₋₁₀ cycloalkyl;
R² is hydrogen, hydroxyl, halo, C₁₋₈ alkyl, C₃₋₁₀ cycloalkyl C₁₋₈ alkoxy, C₁₋₈ alkenyloxy or C₁₋₈ alkynyloxy;
R³, R⁴, R⁵ and R⁶ are, independently, hydrogen, hydroxyl, halo, cyano, nitro, amino, mono- and bis-C₁₋₈ alkyl amino, C₁₋₈ alkyl, C₂₋₈ alkenyl, C₂₋₈ alkynyl, C₁₋₈ haloalkyl, C₁₋₈ alkoxy, C₁₋₈ haloalkoxy, C₁₋₈ alkylthio or C₃₋₁₀ cycloalkyl;
A is halo, C₁₋₁₀ alkyl, C₂₋₁₀ alkenyl, C₂₋₁₀ alkynyl, C₁₋₈ haloalkyl, C₁₋₈ alkoxy, C₃₋₁₀ cycloalkyl, C₃₋₁₀ cycloalkyloxy, aryl, arylalkyl, aryloxy, arylalkyloxy or arylthio; preferably A is halo, C₁₋₈ alkyl, C₂₋₈ alkenyl, C₂₋₈ alkynyl, C₁₋₈ haloalkyl, C₁₋₈ alkoxy, C₃₋₁₀ cycloalkyl, C₃₋₁₀ cycloalkyloxy, aryl, arylalkyl, aryloxy, arylalkyloxy or arylthio;
or a salt or a N-oxide thereof, provided that if A is methyl and each R¹, R³, R⁴, R⁵ and R⁶ is hydrogen R² is not chlorine.

Unless otherwise stated, the substituents are unsubstituted or substituted, preferably the substituents are unsubstituted or substituted by the substituents given below. Unless otherwise stated, the following terms used in the specification and claims have the meanings given below:

“Alkyl” means a linear saturated monovalent hydrocarbon radical of one to eight carbon atoms or a branched saturated monovalent hydrocarbon radical of three to ten carbon atoms, or the number of carbon atoms as indicated, e.g. methyl, ethyl, n-propyl, iso-propyl, n-butyl, sec-butyl, iso-butyl, tert-butyl, n-pentyl, iso-amyl, n-hexyl and the like. It is noted that this definition applies both when the term is used alone and when it is used as part of a compound term, such as “haloalkyl” and similar terms. Preferably, linear alkyl groups contain one to six carbon atoms, more preferably one to three carbon atoms and most preferably are selected from methyl, ethyl or n-propyl. Preferably, branched alkyl groups contain three to six carbon atoms and more preferably are selected from iso-propyl(1-methylethyl), sec-butyl(1-methylpropyl), iso-butyl(2-methylpropyl), tert-butyl(1,1-dimethylethyl) or iso-amyl(3-methylbutyl).

“Cycloalkyl” means a monovalent cyclic hydrocarbon radical of three to eight ring carbons and, more preferably, three to six ring carbons. Cycloalkyl groups are fully saturated. Preferably, cycloalkyl groups are selected from cyclopropyl, cyclobutyl, cyclopentyl and cyclohexyl.

“Heterocyclic” means a heterocyclic moiety containing at least one atom of carbon, and at least one element other than carbon, such as sulfur, oxygen or nitrogen within a ring structure. These structures may comprise either simple aromatic rings or non-aromatic rings. Some examples are pyridine, pyrimidine and dioxane.

“Alkenyl” means a linear monovalent saturated hydrocarbon radical of two to eight carbon atoms, or a branched monovalent hydrocarbon radical of three to eight carbon atoms containing at least one double bond, e.g. ethenyl, propenyl and the like. Where appropriate, an alkenyl group can be of either the (E)- or (Z)-configuration. Preferably, linear alkenyl groups contain two to six carbon atoms and more preferably are selected from ethenyl, prop-1-enyl, prop-2-enyl, prop-1,2-dienyl, but-1-enyl, but-2-enyl, but-3-enyl, but-1,2-dienyl, but-1,3-dienyl, pent-1-enyl, pent-3-enyl and hex-1-enyl. Preferably, branched alkenyl groups contain three to six carbon atoms and more preferably are selected from 1-methylethenyl, 1-methylprop-1-enyl, 1-methylprop-2-enyl, 2-methylprop-1-enyl, 2-methylprop-2-enyl and 4-methyl-pent-3-enyl.

“Cycloalkenyl” means a monovalent cyclic hydrocarbon radical of three to eight ring carbons and, more preferably, three to six ring carbons containing at least one double bond. Preferably, cycloalkenyl groups are selected from cyclopropenyl, cyclobutenyl, cyclopentenyl and cyclohexenyl.

“Alkynyl” means a linear monovalent saturated hydrocarbon radical of two to eight carbon atoms, or a branched monovalent hydrocarbon radical of five to eight carbon atoms, containing at least one triple bond, e.g. ethynyl, propynyl and the like. Preferably, linear alkynyl groups contain two to six carbon atoms and more preferably are selected from ethynyl, prop-1-ynyl, prop-2-ynyl, but-1-ynyl, but-2-ynyl and but-3-ynyl. Preferably, branched alkynyl groups contain four to six carbon atoms and more preferably are selected from 1-methylprop-2-ynyl, 3-methylbut-1-ynyl, 1-methylbut-2-ynyl, 1-methylbut-3-ynyl and 1-methylbut-3-ynyl.

“Alkoxy” means a radical —OR, where R is alkyl, alkenyl or alkynyl as defined above and, preferably, wherein R is alkyl. Alkoxy groups include, but are not limited to, methoxy, ethoxy, 1-methylethoxy, propoxy, butoxy, 1-methylpropoxy and 2-methylpropoxy. Preferably alkoxy means methoxy or ethoxy.

“Alkenoxy” means a radical —OR, where R is alkenyl as defined above.

“Alkynoxy” means a radical —OR, where R is alkynyl as defined above.

“Cycloalkyloxy” means a radical —OR, where R is cycloalkyl as defined above.

“Alkoxyalkyl” means a radical —ROR, where each R is, independently, alkyl as defined above

“Aryl” or “aromatic ring moiety” refers to an aromatic substituent which may be a single ring or multiple rings which are fused together, linked covalently, thus aryl groups derived from arenes by removal of a hydrogen atom from a ring carbon atom, and arenes are monocyclic and polycyclic aromatic hydrocarbons. The term “Aryl” may mean substituted or unsubstituted aryl unless otherwise indicated and hence the aryl moieties may be unsubstituted or substituted with one or more of the same or different substituents. Representative examples of aryl include, for example, phenyl, naphthyl, azulenyl, indanyl, indenyl, anthracenyl, phenanthrenyl, tetrahydronaphthyl, biphenyl, diphenylmethyl and 2,2-diphenyl-1-ethyl. therefore

Suitably, substituents for “aryl” groups may be selected from the list including aryl, cycloalkyl, cycloalkenyl and heterocyclic moiety containing at least one atom of carbon, and at least one element other than carbon, such as sulfur, oxygen or nitrogen within a ring structure, halogen, alkyl, haloalkyl, cycloalkyl, cycloalkylalkyl, alkenyl, haloalkenyl, cycloalkenyl, alkynyl, haloalkynyl, alkoxy, haloalkoxy, cycloalkyloxy, haloalkenyloxy, haloalkynyloxy, alkylthio, haloalkylthio, cycloalkylthio, alkylcarbonyl, haloalkylcarbonyl, cycloalkylcarbonyl, alkenylcarbonyl, alkynylcarbonyl, formyl, alkoxyalkyl, cyano, nitro, hydroxy, mercapto, amino, alkylamino, dialkylamino, —C(O)(C₁₋₄ alkoxy), —C(O)NH₂, —C(O)NH(C₁₋₄ alkyl), —C(O)N(C₁₋₄ alkyl)(C₁₋₄ alkyl), —OC(O)NH(C₁₋₄ alkyl), —OC(O)N(C₁₋₄ alkyl)(C₁₋₄ alkyl), —NHC(O)(C₁₋₄ alkyl), —NHC(O)(C₁₋₄ alkoxy), —N(C₁₋₄ alkyl)C(O)(C₁₋₄ alkyl), —N(C₁₋₄ alkyl)C(O)(C₁₋₄ alkoxy), —OC(O)(C₁₋₄ alkyl), —OC(O)(C₁₋₄alkoxy), —Si(C₁₋₄ alkyl)₃, —Si(C₁₋₄ alkoxy)₃, and aryloxy. Preferred substituents are aryl, cycloalkyl, cycloalkenyl and heterocyclic moiety containing at least one atom of carbon, and at least one element other than carbon, such as sulfur, oxygen or nitrogen within a ring structure, alkyl, alkenyl, alkynyl, cycloalkyl, halo, haloalkyl, alkoxy, haloalkoxy, nitro and cyano and are more preferably halogen (in particular, fluoro or chloro), cyano, alkyl (in particular, methyl and ethyl), haloalkyl (in particular, trifluoromethyl), alkoxy (in particular, methoxy or ethoxy) and haloalkoxy.

The aryl, cycloalkyl, cycloalkenyl or heterocyclic substituent of the aryl, cycloalkyl, cycloalkenyl or heterocyclic group may be unsubstituted or further substituted, wherein the substituents are selected from the list including halogen, alkyl, haloalkyl, cycloalkyl, cycloalkylalkyl, alkenyl, haloalkenyl, cycloalkenyl, alkynyl, haloalkynyl, alkoxy, haloalkoxy, cycloalkyloxy, haloalkenyloxy, haloalkynyloxy, alkylthio, haloalkylthio, cycloalkylthio, alkylcarbonyl, haloalkylcarbonyl, cycloalkylcarbonyl, alkenylcarbonyl, alkynylcarbonyl, alkoxycarbonyl, alkoxyalkyl, cyano, nitro, hydroxy, mercapto, amino, alkylamino and dialkylamino. Preferred aryl substituent of the aryl group may be unsubstituted aryl or aryl substituted by substituents selected from the list including halogen, alkyl, alkenyl, alkynyl, cycloalkyl, halo, haloalkyl, alkoxy, haloalkoxy and cyano and are more preferably halogen (in particular, fluoro or chloro), cyano, alkyl (in particular, methyl and ethyl), haloalkyl (in particular, trifluoromethyl), alkoxy (in particular, methoxy or ethoxy) and haloalkoxy.

Typical examples for unsubstituted or substituted aryl include 2-fluorophenyl, 3-fluorophenyl, 4-fluorophenyl, 2-chlorophenyl, 3-chlorophenyl, 4-chlorophenyl, 2-bromophenyl, 3-bromophenyl, 4-bromophenyl, 2-methylphenyl, 3-methylphenyl, 4-methylphenyl, 2-methoxyphenyl, 3-methoxyphenyl, 4-methoxyphenyl, 2-cyanophenyl, 3-cyanophenyl, 4-cyanophenyl, 2-trifluoromethylphenyl, 3-trifluoromethylphenyl, 4-trifluoromethylphenyl, 2-trifluoromethoxyphenyl, 3-trifluoromethoxyphenyl, 4-trifluoromethoxyphenyl, 2,3-difluorophenyl, 2,4-difluorophenyl, 2,5-difluorophenyl, 2,6-difluorophenyl, 3,4-difluorophenyl, 3,5-difluorophenyl, 2,3-dichlorophenyl, 2,4-dichlorophenyl, 2,5-dichlorophenyl, 2,6-dichlorophenyl, 3,4-dichlorophenyl, 3,5-dichlorophenyl, 2,3-dibromophenyl, 2,4-dibromophenyl, 2,5-dibromophenyl, 2,6-dibromophenyl, 3,4-dibromophenyl, 3,5-dibromophenyl, 2,3-dimethylphenyl, 2,4-dimethylphenyl, 2,5-dimethylphenyl, 2,6-dimethylphenyl, 3,4-dimethylphenyl, 3,5-dimethylphenyl, 2,3-dimethoxyphenyl, 2,4-dimethoxyphenyl, 2,5-dimethoxyphenyl, 2,6-dimethoxyphenyl, 3,4-dimethoxyphenyl, 3,5-dimethoxyphenyl, 2,3-dicyanophenyl, 2,4-dicyanophenyl, 2,5-dicyanophenyl, 2,6-dicyanophenyl, 3,4-dicyanophenyl, 3,5-dicyanophenyl, 2,3-di(trifluoromethyl)phenyl, 2,4-di(trifluoromethyl)phenyl, 2,5-di(trifluoromethyl)phenyl, 2,6-di(trifluoromethyl)phenyl, 3,4-di(trifluoromethyl)phenyl, 3,5-di(trifluoromethyl)phenyl, 2,3-di(trifluoromethoxy)phenyl, 2,4-di(trifluoromethoxy)phenyl, 2,5-di(trifluoromethoxy)phenyl, 2,6-di(trifluoromethoxy)phenyl, 3,4-di(trifluoromethoxy)phenyl, 3,5-di(trifluoromethoxy)phenyl, 4-chloro-3-fluorophenyl, 3-fluoro-4-methylphenyl, 3-fluoro-4-methoxyphenyl, 3-chloro-4-fluorophenyl, 3-chloro-4-methylphenyl, 3-chloro-4-methoxyphenyl, 4-fluoro-3-methylphenyl, 4-chloro-3-methylphenyl, 4-methoxy-3-methylphenyl, 4-fluoro-3-methoxyphenyl, 4-chloro-3-methoxyphenyl, 3-methoxy-4-methylphenyl, 3-chloro-5-fluorophenyl, 3-chloro-5-methylphenyl, 3-chloro-5-methoxyphenyl, 3-fluoro-5-methylphenyl, 3-fluoro-5-methoxyphenyl, 3-methoxy-5-methylphenyl.

“Halo” or “halogen” means fluoro, chloro, bromo or iodo, preferably chloro or fluoro.

“Haloalkyl” means alkyl as defined above substituted with one or more of the same or different halo atoms. Therefore this definition of haloalkyl may also include perhalogenated alkyl groups. Examples of haloalkyl groups include, but are not limited to chloromethyl, fluoromethyl, dichloromethyl, difluoromethyl, trichloromethyl, trifluoromethyl, 2-fluoroethyl, 2-trifluoroethyl, 1-difluoroethyl, 2-trifluoro-1-difluoroethyl, 2-chloro-ethyl, 2-trichloro-1-dichloroethyl 2-iodoethyl, 3-fluoropropyl, 3-chloropropyl, 2-trifluoro-1-chloroethyl and 1-difluoro-2-difluoro-3-trifluoropropyl.

“Haloalkenyl” means alkenyl as defined above substituted with one or more of the same or different halo atoms.

“Haloalkynyl” means alkynyl as defined above substituted with one or more of the same or different halo atoms.

“Haloalkoxy” means a radical —OR, wherein R is haloalkyl as defined above.

“Haloalkenyloxy” means a radical —OR, wherein R is haloalkenyl as defined above.

“Haloalkynyloxy” means a radical —OR, wherein R is haloalkynyl as defined above.

“Arylalkyl” means a radical —R^{a}R^{b} where R^{a} is an alkylene group and R^{b} is an unsubstituted or substituted aryl group as defined above; “Arylalkenyl” means a radical —R^{a}R^{b} where R^{a} is an alkenylene group as defined below and R^{b} is an unsubstituted or substituted aryl group as defined above; “Arylalkynyl” means a radical —R^{a}R^{b} where R^{a} is an alkynylene group as defined below and R^{b} is an unsubstituted or substituted aryl group as defined above. An example of an arylalkyl group is the benzyl group. When R^{a} is an alkylene group or an alkenylene group or an alkynylene, this group may also be substituted with one or more of the same or different substitutents, suitably, the substituents being as defined above for “aryl”.

“Cycloalkylalkyl” means a radical —R^{a}R^{b} where R^{a} is an alkylene group, as defined below and R^{b} is a cycloalkyl group as defined above.

“Cycloalkylalkenyl” means a radical —R^{a}R^{b} where R^{a} is a an alkenylene group as defined below and R^{b} is a cycloalkyl group as defined above.

“Cycloalkylalkenyl” means a radical —R^{a}R^{b} where R^{a} is an alkynylene group as defined below and R^{b} is a cycloalkyl group as defined above.

“Alkylene” means a linear saturated divalent hydrocarbon radical of one to six carbon atoms or a branched saturated divalent hydrocarbon radical of three to six carbon atoms, e.g. methylene, ethylene, propylene, 2-methylpropylene and the like. Preferred alkylene groups are the divalent radicals of the alkyl groups defined above.

“Alkenylene” means a linear divalent hydrocarbon radical of two to six carbon atoms or a branched divalent hydrocarbon radical of three to six carbon atoms, containing at least one double bond, e.g. ethenylene, propenylene and the like. Preferred alkenylene groups are the divalent radicals of the alkenyl groups defined above.

“Alkynylene” means a linear divalent hydrocarbon radical of two to six carbon atoms or a branched divalent hydrocarbon radical of three to six carbon atoms, containing at least one triple bond, e.g. ethynylene, propynylene and the like. Preferred alkynylene groups are the divalent radicals of the alkynyl groups defined above.

“Aryloxy” means a radical —OR, wherein R is an aryl group as defined above.

“Arylalkyloxy” means a radical —OR wherein R is an arylalkyl group as defined above.

“Arylalkenyleneoxy” means a radical —OR wherein R is an arylalkenylene group as defined above.

“Arylalkynyleneoxy” means a radical —OR wherein R is an arylalkynylenel group as defined above.

“Alkylthio” means a radical —SR, where R is an alkyl as defined above. Alkylthio groups include, but are not limited to, methylthio, ethylthio, propylthio, tert-butylthio, hexylthio, and the like.

“Alkenylthio” means a radical —SR, where R is an alkenyl as defined above.

“Alkynylthio” means a radical —SR, where R is an alkynyl as defined above.

“Cycloalkylthio” means a radical —SR, where R is a cycloalkyl group as defined above.

“Haloalkylthio” means a radical —SR, where R is a haloalkyl group as defined above.

“Arylthio” means a radical —SR, where R is an aryl group as defined above

“Alkylcarbonyl” means a radical —C(O)R, wherein R is alkyl as defined above.

“Alkenylcarbonyl” means a radical —C(O)R, wherein R is alkenyl as defined above.

“Alkynylcarbonyl” means a radical —C(O)R, wherein R is alkynyl as defined above.

“Cycloalkylcarbonyl” means a radical —C(O)R, wherein R is cycloalkyl as defined above.

“Alkoxycarbonyl” means a radical —C(O)OR, wherein R is alkyl as defined above.

“Haloalkylcarbonyl” means a radical —C(O)R, wherein R is haloalkyl as defined above.

“Cyano” means a —CN group.

“Hydroxy” or “hydroxyl” means an —OH group.

“Nitro” means an —NO₂ group.

“Amino” means an —NH₂ group.

“Alkylamino” means a radical —NRH, where R is alkyl as defined above.

“Dialkylamino” means a radical —NRR, where each R is, independently, alkyl as defined above.

“Mercapto” means an —SH group.

The groups defined above (as already noted for ‘aryl’ and ‘arylalkyl’ groups) when used alone or as part of a compound term (e.g. alkyl when used alone or as part of, for example, haloalkyl) may be unsubstituted or substituted by one or more substituents. In particular, alkyl, alkenyl, alkynyl, cycloalkyl, cycloalkenyl, alkoxy, cycloalkyloxy, haloalkyl, haloalkoxy, alkylthio, aryl, arylalkyl, aryloxy and arylalkyloxy groups may be unsubstituted or substituted.

Suitably, these optional substituents are independently selected from halogen, alkyl, haloalkyl, cycloalkyl, cycloalkylalkyl, alkenyl, haloalkenyl, cycloalkenyl, alkynyl, haloalkynyl, alkoxy, haloalkoxy, cycloalkyloxy, haloalkenyloxy, haloalkynyloxy, alkylthio, haloalkylthio, cycloalkylthio, formyl, alkylcarbonyl, haloalkylcarbonyl, cycloalkylcarbonyl, alkenylcarbonyl, alkynylcarbonyl, alkoxyalkyl, cyano, nitro, hydroxy, mercapto, amino, alkylamino, dialkylamino, aryl, cycloalkyl, cycloalkenyl and heterocyclic moiety containing at least one atom of carbon, and at least one element other than carbon, such as sulfur, oxygen or nitrogen within a ring structure, —C(O)(C₁₋₄alkoxy), —C(O)NH₂, —C(O)NH(C₁₋₄ alkyl), —C(O)N(C₁₋₄ alkyl)(C₁₋₄ alkyl), —OC(O)NH(C₁₋₄ alkyl), —OC(O)N(C₁₋₄ alkyl)(C₁₋₄ alkyl), —NHC(O)(C₁₋₄ alkyl), —NHC(O)(C₁₋₄ alkoxy), —N(C₁₋₄ alkyl)C(O)(C₁₋₄ alkyl), —N(C₁₋₄ alkyl)C(O)(C₁₋₄ alkoxy), —OC(O)(C₁₋₄ alkyl), —OC(O)(C₁₋₄ alkoxy), —Si(C₁₋₄ alkyl)₃, —Si(C₁₋₄ alkoxy)₃, and aryloxy. Preferred substituents are alkyl, alkenyl, alkynyl, cycloalkyl, halo, haloalkyl, alkoxy, haloalkoxy and cyano and are more preferably halogen (in particular, fluoro or chloro), cyano, alkyl (in particular, methyl and ethyl), haloalkyl (in particular, trifluoromethyl), alkoxy (in particular, methoxy or ethoxy), haloalkoxy, aryl, cycloalkyl, cycloalkenyl and heterocyclic moiety containing at least one atom of carbon, and at least one element other than carbon, such as sulfur, oxygen or nitrogen within a ring structure.

The compounds of formula I may exist in different geometric or optical isomeric forms or in different tautomeric forms. One or more centres of chirality may be present, in which case compounds of the formula I may be present as pure enantiomers, mixtures of enantiomers, pure diastereomers or mixtures of diastereomers. There may be double bonds present in the molecule, such as C═C or C═N bonds, in which case compounds of formula I may exist as single isomers or mixtures of isomers. Centres of tautomerisation may be present. This invention covers all such isomers and tautomers and mixtures thereof in all proportions as well as isotopic forms such as deuterated compounds. Also atropisomerism may occur as a result of a restricted rotation about a single bond.

Suitable salts of the compounds of formula I include acid addition salts such as those with an inorganic acid such as hydrochloric, hydrobromic, sulphuric, nitric or phosphoric acid, or an organic carboxylic acid such as oxalic, tartaric, lactic, butyric, toluic, hexanoic or phthalic acid, or a sulphonic acid such as methane, benzene or toluene sulphonic acid. Other examples of organic carboxylic acids include haloacids such as trifluoroacetic acid.

N-oxides are oxidised forms of tertiary amines or oxidised forms of nitrogen containing heteroaromatic compounds. They are described in many books for example in “Heterocyclic N-oxides” by Angelo Albini and Silvio Pietra, CRC Press, Boca Raton, Fla., 1991.

In particularly preferred embodiments of the invention, the preferred groups for R¹ to R⁶ and A in any combination thereof, are as set out below.

In one embodiment according to formula (I), R¹ is hydrogen, halo, cyano, C₁₋₈ alkyl, C₁₋₈ alkoxy, C₁₋₈ alkenyloxy, C₁₋₈ alkynyloxy, C₁₋₈ haloalkyl, or C₁₋₈ alkylthio. In a further embodiment, R¹ is hydrogen, halo, C₁₋₃ alkyl, C₁₋₃ alkoxy, C₁₋₃ alkenyloxy, C₁₋₃ alkynyloxy, C₁₋₃ haloalkyl, or C₁₋₃ alkylthio.

In a further embodiment according to formula (I), R¹ is hydrogen, halo, C₁₋₃ alkyl, C₁₋₃ alkoxy, C₁₋₃ alkenyloxy, C₁₋₃ alkynyloxy, or C₁₋₄ haloalkyl. In a still further embodiment, R¹ is hydrogen, fluoro, chloro, methyl, ethyl, methoxy, ethoxy or trifluoromethyl and, more preferably, hydrogen, methyl or methoxy.

In one embodiment, R² is hydrogen according to formula (I), hydroxyl, halo, C₁₋₅ alkyl C₁₋₅ alkoxy, C₁₋₅ alkenyloxy or C₁₋₅ alkynyloxy. In a further embodiment, R² is hydrogen, hydroxyl, chloro, methyl or methoxy and, more preferably, hydrogen, methyl or methoxy.

In one embodiment according to formula (I), R³, R⁴, R⁵ and R⁶ are, independently, hydrogen, halo, cyano, C₁₋₈ alkyl, C₁₋₈ haloalkyl, C₁₋₈ alkoxy, C₁₋₈ alkenyloxy, C₁₋₈ alkynyloxy, or C₁₋₈ haloalkoxy. In a further embodiment, R³, R⁴, R⁵ and R⁶ are, independently, hydrogen, halo, cyano, C₁₋₃ alkyl, C₁₋₃ haloalkyl, C₁₋₃ alkoxy, C₁₋₃ alkenyloxy, C₁₋₃ alkynyloxy, or C₁₋₃ haloalkoxy. In a further embodiment, R³, R⁴, R⁵ and R⁶ are, independently, hydrogen, halo, cyano, C₁₋₃ alkyl or C₁₋₃ alkoxy, C₁₋₃ alkenyloxy, C₁₋₃ alkynyloxy. In a still further embodiment, R³, R⁴, R⁵ and R⁶ are, independently, hydrogen, bromo, cyano, chloro, fluoro, methyl or methoxy.

In one embodiment according to formula (I), A is halo, C₁₋₈ haloalkyl, unsubstituted or substituted aryl, unsubstituted or substituted arylalkyl or unsubstituted or substituted aryloxy. In a further embodiment, A is halo, unsubstituted or substituted phenyl, unsubstituted or substituted naphthyl, unsubstituted or substituted benzyl, unsubstituted or substituted phenoxy, unsubstituted or substituted phenylthio or unsubstituted or substituted arylethynyl (in particular, phenylethynyl). In a further embodiment, A is halogen, unsubstituted or substituted phenyl, unsubstituted or substituted benzyl or unsubstituted or substituted phenoxy and, more preferably, unsubstituted or substituted phenyl and unsubstituted or substituted benzyl. Suitable substituents are as defined above but, more suitably, may be halo, cyano, nitro, hydroxyl, C₁₋₃ alkyl, C₁₋₃ haloalkyl, C₁₋₃ alkoxy, C₁₋₃ alkylcarbonyl, C₁₋₃ alkoxycarbonyl or a combination of any of these substituents or, even more suitably, chloro, fluoro, methyl, trifluoromethyl or methoxy or a combination of any of these substituents.

In one more preferred embodiment according to formula (I), R¹ is hydrogen, halo, cyano, C₁₋₃ alkyl, C₁₋₃ alkoxy, C₁₋₃ haloalkyl, or C₁₋₃ alkylthio; R² is hydrogen, hydroxyl, halo, C₁₋₅ alkyl, C₃₋₅ cycloalkyl, C₁₋₅ alkynyloxy or C₁₋₅ alkoxy; R³, R⁴, R⁵ and R⁶ are, independently, hydrogen, halo, hydroxyl, cyano, C₁₋₈ alkyl, C₁₋₈ haloalkyl, C₁₋₈ alkoxy, C₁₋₈ haloalkoxy, amino or mono- or di-C₁₋₈ alkyl amino and A is halo, C₁₋₈ alkyl, C₂₋₈ alkenyl, C₂₋₈ alkynyl, C₁₋₈ haloalkyl, C₁₋₈ alkoxy, C₃₋₁₀ cycloalkyl, C₃₋₁₀ cycloalkyloxy, aryl, arylalkyl, aryloxy, arylalkyloxy or arylthio;

In one even more preferred embodiment according to formula (I), R¹ is hydrogen, fluoro, chloro, methyl, ethyl, methoxy, ethoxy or trifluoromethyl, preferably hydrogen, methyl or methoxy. R² is hydrogen, hydroxyl, chloro, methyl or methoxy, preferably hydrogen, methyl or methoxy; R³, R⁴, R⁵ and R⁶ are, independently, hydrogen, halo, cyano, C₁₋₃ alkyl, C₁₋₃ haloalkyl, C₁₋₃ alkoxy, C₁₋₃ haloalkoxy, amino or mono- or di-C₁₋₈ alkyl amino, preferably independently, hydrogen, halo, cyano, C₁₋₃ alkyl or C₁₋₃ alkoxy, more preferably independently, hydrogen, halo, cyano, C₁₋₃ alkyl or C₁₋₃ alkoxy; A is halo, C₁₋₈ alkyl, unsubstituted or substituted aryl, unsubstituted or substituted arylalkyl or unsubstituted or substituted aryloxy, preferably halo, unsubstituted or substituted phenyl, unsubstituted or substituted naphthyl, unsubstituted or substituted benzyl, unsubstituted or substituted phenoxy, unsubstituted or substituted phenylthio or unsubstituted or substituted arylethynyl, more preferably unsubstituted or substituted phenyl, unsubstituted or substituted naphthyl, unsubstituted or substituted benzyl, unsubstituted or substituted phenoxy, unsubstituted or substituted phenylthio or unsubstituted or substituted arylethynyl.

In a preferred embodiment according to formula (I), R¹ is hydrogen, halo, C₁₋₃ alkyl, C₁₋₃ haloalkyl or C₁₋₃ alkoxy, R² is hydrogen, hydroxyl, halo, C₁₋₅ alkyl, C₃₋₅ cycloalkyl or C₁₋₅ alkoxy, R³, R⁴, R⁵ and R⁶ are, independently hydrogen, halo, C₁₋₃ alkyl, C₁₋₃ haloalkyl or C₁₋₃ alkoxy and A is halo, unsubstituted or substituted aryl, unsubstituted or substituted arylalkyl, unsubstituted or substituted aryloxy or unsubstituted or substituted arylthio, wherein the optional substituents are selected from halo, cyano, nitro, hydroxyl, C₁₋₃ alkyl, C₁₋₃ haloalkyl, C₁₋₃ alkylcarbonyl, C₁₋₃ alkoxycarbonyl and C₁₋₃ alkoxy or a combination of any of these substituents.

In a more preferred embodiment according to formula (I), R¹ is hydrogen, fluoro, chloro, methyl, ethyl, trifluoromethyl, ethoxy or methoxy, preferably hydrogen, fluoro, chloro, methyl, ethyl, ethoxy or methoxy, R² is hydrogen, chloro, methyl or methoxy, R³, R⁴, R⁵ and R⁶ are, independently, hydrogen, fluoro, chloro, methyl, hydroxyl, trifluoromethyl or methoxy and A is bromo, chloro, iodo, unsubstituted or substituted phenyl, unsubstituted or substituted phenylmethyl, unsubstituted or substituted phenoxy, unsubstituted or substituted phenylthio or unsubstituted or substituted phenylethynyl, wherein the optional substituents are selected from fluoro, chloro, cyano, methyl, trifluoromethyl or methoxy or a combination of any of these substituents.

In a most preferred embodiment according to formula (I) A is halogen, unsubstituted or substituted phenyl, unsubstituted or substituted benzyl or unsubstituted or substituted phenoxy, especially A is unsubstituted or substituted phenyl and unsubstituted or substituted benzyl.

Accordingly, the preferred compound of formula I of the present invention is a compound of formula (I′):

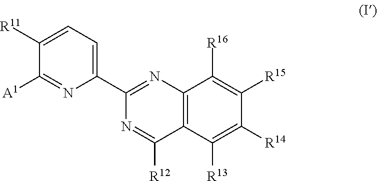

wherein:
R¹¹ is hydrogen, hydroxyl, halo, cyano, unsubstituted C₁₋₈ alkyl, substituted C₁₋₈ alkyl, C₁₋₈ haloalkyl, unsubstituted C₁₋₈ alkoxy, substituted C₁₋₈ alkoxy, C₁₋₈ haloalkoxy, unsubstituted C₁₋₈ alkylthio, substituted C₁₋₈ alkylthio, unsubstituted C₃₋₁₀ cycloalkyl or substituted C₃₋₁₀ cycloalkyl;
R¹² is hydrogen, hydroxyl, halo, unsubstituted C₁₋₈ alkyl, substituted C₁₋₈ alkyl, substituted C₃₋₁₀ cycloalkyl, unsubstituted C₃₋₁₀ cycloalkyl, C₁₋₈ haloalkyl, unsubstituted C₁₋₈ alkoxy, substituted C₁₋₈ alkoxy, unsubstituted C₂₋₈ alkenyloxy, substituted C₂₋₈ alkenyloxy, unsubstituted C₂₋₈ alkynyloxy; or substituted C₂₋₈ alkynyloxy;
R¹³, R¹⁴, R¹⁵ and R¹⁶ are, independently, hydrogen, hydroxyl, halo, cyano, nitro, —NR¹⁷R¹⁸ where R¹⁷ and R¹⁸ are independently H, C₁₋₄alkyl or substituted C₁₋₄alkyl or combine with the interjacent nitrogen to form a five- or six-membered ring which may comprise one or two or three heteroatoms (one or two N, O or S atoms in addition to the interjacent nitrogen atom), in which case the heterocyclic ring is unsubstituted or the heterocyclic ring is substituted by one or two C₁₋₄alkyl groups, unsubstituted C₁₋₈ alkyl, substituted C₁₋₈ alkyl, unsubstituted C₂₋₈ alkenyl, substituted C₂₋₈ alkenyl, unsubstituted C₂₋₈ alkynyl, substituted C₂₋₈ alkynyl, C₁₋₈ haloalkyl, unsubstituted C₁₋₈ alkoxy, substituted C₁₋₈ alkoxy, C₁₋₈ haloalkoxy, unsubstituted C₁₋₈ alkylthio, substituted C₁₋₈ alkylthio, unsubstituted C₃₋₁₀ cycloalkyl or substituted C₃₋₁₀ cycloalkyl;
A¹ is halo, unsubstituted C₁₋₈ alkyl, substituted C₁₋₈ alkyl, unsubstituted C₂₋₁₀ alkenyl, substituted C₂₋₈ alkenyl, unsubstituted C₂₋₈ alkynyl, substituted C₂₋₈ alkynyl, C₁₋₈ haloalkyl, unsubstituted C₁₋₈ alkoxy, substituted C₁₋₈ alkoxy, unsubstituted C₃₋₁₀ cycloalkyl, substituted C₃₋₁₀ cycloalkyl, unsubstituted C₃₋₁₀ cycloalkyloxy, substituted C₃₋₁₀ cycloalkyloxy, unsubstituted aryl, substituted aryl, unsubstituted arylalkyl, substituted arylalkyl, unsubstituted arylalkenyl, substituted arylalkenyl, unsubstituted arylalkynyl, substituted arylalkynyl, unsubstituted aryloxy, substituted aryloxy, unsubstituted arylalkyloxy, substituted arylalkyloxy, unsubstituted arylthio or substituted arylthio;
or a salt or a N-oxide thereof, provided that if A¹ is methyl and each R¹¹, R¹³, R¹⁴, R¹⁵ and R¹⁶ is hydrogen R¹² is not chlorine.

The alkyl groups, the alkenyl groups, the alkynyl groups and the alkoxy group in the compound of formula (I′) are either linear or branched.

The preferred substituents of the substituted alkyl groups, the substituted alkenyl groups, the substituted alkynyl groups and the substituted alkoxy group in the compound of formula (I′) are selected from the following substituents F, Cl, Br, I, —OH, —CN, nitro, —C₁₋₄alkoxy, —C₁₋₄ alkylthio, —NR¹⁷R¹⁸ where R¹⁷ and R¹⁸ are independently H, —C₁₋₄alkyl or substituted —C₁₋₄alkyl or combine with the interjacent nitrogen to form a five- or six-membered ring which may comprise one or two or three heteroatoms (one or two N, O or S atoms in addition to the interjacent nitrogen atom), in which case the heterocyclic ring is unsubstituted or the heterocyclic ring is substituted by one or two C₁₋₄ alkyl groups, —C(O)H, —C(O)(C₁₋₄ alkyl), —C(O)(C₁₋₄ alkoxy), —C(O)NH₂, —C(O)NH(C₁₋₄ alkyl), —C(O)N(C₁₋₄ alkyl)(C₁₋₄ alkyl), —OC(O)NH(C₁₋₄ alkyl), —OC(O)N(C₁₋₄ alkyl)(C₁₋₄ alkyl), —NHC(O)(C₁₋₄ alkyl), —NHC(O)(C₁₋₄ alkoxy), —N(C₁₋₄ alkyl)C(O)(C₁₋₄ alkyl), —N(C₁₋₄ alkyl)C(O)(C₁₋₄ alkoxy), —OC(O)(C₁₋₄ alkyl), —OC(O)(C₁₋₄alkoxy), —Si(C₁₋₄ alkyl)₃, —Si(C₁₋₄ alkoxy)₃, aryl, aryloxy, —(C₁₋₈-perhaloalkyl), arylC₁₋₄alkynyl, —C₁₋₆alkynyl, wherein the alkyl, alkenyl, alkynyl, alkoxy, aryl groups are either substituted or unsubstituted, preferably these substituents of the substituted groups bear only one further substituent, more preferably are these substituents of the substituted groups not further substituted.

The more preferred substituents of the substituted C₁ to C₄ alkyl groups are selected from the following substituents —OH, CN, F, Cl, C₁₋₄alkoxy, C₁₋₄alkylamino. The alkyl groups are branched or linear. The most preferred alkyl groups are methyl, ethyl, propyl, iso-propyl, butyl, iso-butyl (2-methylpropyl), pentyl, 1-methylpentyl, 1-ethylpentyl, iso-pentyl (3-methylbutyl), hexyl, heptyl, octyl, or nonyl.

Preferably the alkyl groups in the compound of formula (I′) and/or the alkoxy groups in the compound of formula (I′) bear not more than two further substituents, more preferably the alkyl groups in the compound of formula (I′) and/or the alkoxy groups in the compound of formula (I′) bear not more than one further substituent, most preferred the alkyl groups in the compound of formula (I′) and/or the alkoxy groups in the compound of formula (I′) are not further substituted.

In the preferred compounds of the formula (I′) the preferred alkyl groups and the preferred alkoxy groups are methyl, ethyl, propyl, methoxy and ethoxy groups. Methyl, ethyl and methoxy groups are very particularly preferred.

The preferred substituents in the compound of formula (I′) of the substituted aryl groups in the compound of formula (I′) are selected from the following substituents F, Cl, Br, I, —OH, —CN, nitro, —C₁₋₄ alkyl, —C₁₋₄ alkoxy, C₁₋₄ alkenyloxy, —C₁₋₄ alkynyloxy, —C₁₋₄ alkoxyC₁₋₄ alkyl, —C₁₋₄ alkylthio, —NR¹⁷R¹⁸ where R¹⁷ and R¹⁸ are independently H, —C₁₋₄alkyl or substituted —C₁₋₄ alkyl or combine with the interjacent nitrogen to form a five- or six-membered ring which may comprise one or two or three heteroatoms (one or two N, O or S atoms in addition to the interjacent nitrogen atom), in which case the heterocyclic ring is unsubstituted or the heterocyclic ring is substituted by one or two —C₁₋₄alkyl groups, —C(O)H, —C(O)(C₁₋₄ alkyl), —C(O)(C₁₋₄ alkoxy), —C(O)NH₂, —C(O)NH(C₁₋₄ alkyl), —C(O)N(C₁₋₄ alkyl)(C₁₋₄ alkyl), —NHC(O)(C₁₋₄alkyl), —N(C₁₋₄ alkyl)C(O)(C₁₋₄ alkyl), —NHC(O)(C₁₋₄ alkoxy), —N(C₁₋₄ alkyl)C(O)(C₁₋₄ alkoxy), —OC(O)NH(C₁₋₄ alkyl), —OC(O)N(C₁₋₄ alkyl) (C₁₋₄ alkyl), —C(O)H, OC(O)(C1-4 alkyl), —OC(O)(C1-4 alkoxy), —Si(C₁₋₄ alkyl)₃, —Si(C₁₋₄ alkoxy)₃, aryl, aryloxy, —(C₁₋₈-perhaloalkyl), —C₁₋₈ alkynyl, wherein the alkyl, alkenyl, alkenyl, aryl groups are either substituted or unsubstituted.

The more preferred substituents of the substituted aryl groups are selected from the following substituents F, Cl, CN, —OH, nitro, —C₁₋₄ alkyl, —C₁₋₄ alkoxy, —C(O)(C₁₋₄ alkoxy), —C(O)H, —C(O)(C₁₋₄ Alkyl) wherein the alkyl groups are either substituted or unsubstituted.

The aryl groups are preferably naphthyl, phenanthrenyl or phenyl groups, more preferably phenyl groups.

The preferred substituents of the substituted aryl groups in the compound of formula (I′) are selected from the following substituents, F, Cl, —C₁₋₄Alkyl, C₁₋₄alkoxy, —CN, —C(O)(C₁₋₄ alkoxy), —C(O)(C₁₋₄ Alkyl).

In formula (I′) preferably
R¹¹ is hydrogen, halo, unsubstituted C₁₋₄ alkyl, substituted C₁₋₄ alkyl, C₁₋₄ haloalkyl, unsubstituted C₁₋₄ alkoxy, substituted C₁₋₄ alkoxy, C₁₋₄ haloalkoxy;
R¹² is hydrogen, hydroxyl, halo, unsubstituted C₁₋₈ alkyl, substituted C₁₋₈ alkyl, unsubstituted C₃₋₁₀ cycloalkyl, substituted C₃₋₁₀ cycloalkyl C₁₋₈ haloalkyl, unsubstituted C₁₋₈ alkoxy, substituted C₁₋₈ alkoxy, unsubstituted C₂₋₈ alkenyloxy, substituted C₂₋₈ alkenyloxy, unsubstituted C₂₋₈ alkynyloxy; or substituted C₂₋₈ alkynyloxy;
R¹³, R¹⁴, R¹⁵ and R¹⁶ are, independently, hydrogen, halo, nitro, amino, unsubstituted C₁₋₄ alkyl, substituted C₁₋₄ alkyl, unsubstituted C₂₋₄ alkenyl, substituted C₂₋₄ alkenyl, unsubstituted C₂₋₄ alkynyl, substituted C₂₋₄ alkynyl, unsubstituted C₁₋₄ alkoxy, substituted C₁₋₄ alkoxy;
A¹ is halo, unsubstituted C₁₋₄ alkyl, substituted C₁₋₄ alkyl, unsubstituted C₂₋₄ alkenyl, substituted C₂₋₄ alkenyl, unsubstituted C₂₋₄ alkynyl, substituted C₂₋₄ alkynyl, C₁₋₄ haloalkyl, unsubstituted C₁₋₄ alkoxy, substituted C₁₋₄ alkoxy, unsubstituted C₃₋₆ cycloalkyl, substituted C₃₋₆ cycloalkyl, unsubstituted C₃₋₆ cycloalkyloxy, substituted C₃₋₆ cycloalkyloxy, unsubstituted aryl, substituted aryl, unsubstituted arylalkyl, substituted arylalkyl, unsubstituted arylalkynyl, substituted arylalkynyl, unsubstituted aryloxy, substituted aryloxy, unsubstituted arylalkyloxy, substituted arylalkyloxy, unsubstituted arylthio or substituted arylthio;
or a salt or a N-oxide thereof, provided that if A¹ is methyl and each R¹¹, R¹³, R¹⁴, R¹⁵ and R¹⁶ is hydrogen R¹² is not chlorine.

More preferably in formula (I′)
R¹¹ is hydrogen, F, Cl, CN, unsubstituted C₁₋₃ alkyl, substituted C₁₋₃alkyl, C₁₋₃ haloalkyl, C₁₋₃ alkoxy;
R¹² is hydrogen, unsubstituted C₁₋₄ alkyl, substituted C₁₋₄ alkyl, C₁₋₄ haloalkyl, unsubstituted C₁₋₄ alkoxy, substituted C₁₋₄ alkoxy;
R¹³, R¹⁴, R¹⁵ and R¹⁶ are, independently, hydrogen, halo, nitro, amino, unsubstituted C₁₋₄ alkyl, substituted C₁₋₄ alkyl, unsubstituted C₂₋₄ alkenyl, substituted C₂₋₄ alkenyl, unsubstituted C₂₋₄ alkynyl, substituted C₂₋₄ alkynyl, unsubstituted C₁₋₄ alkoxy, substituted C₁₋₄ alkoxy;
A¹ is halo, unsubstituted C₁₋₄ alkyl, substituted C₁₋₄ alkyl, unsubstituted aryl, substituted aryl, unsubstituted arylalkyl, substituted arylalkyl, unsubstituted arylalkynyl, substituted arylalkynyl, unsubstituted aryloxy, substituted aryloxy, unsubstituted arylalkyloxy, substituted arylalkyloxy, unsubstituted arylthio or substituted arylthio;
or a salt or a N-oxide thereof.

Preferably at least two of the substituents R¹³, R¹⁴, R¹⁵ and R¹⁶ are H, more preferably at least three of the substituents R¹³, R¹⁴, R¹⁵ and R¹⁶ are H.

More preferably in formula (I′)
R¹¹ is hydrogen, F, Cl, unsubstituted C₁₋₂ alkyl, substituted C₁₋₂ alkyl, C₁₋₂ alkoxy;
R¹² is hydrogen, unsubstituted C₁₋₄ alkyl, substituted C₁₋₄ alkyl, C₁₋₄ haloalkyl, unsubstituted C₁₋₄ alkoxy, substituted C₁₋₄ alkoxy;
R¹³, R¹⁴, R¹⁵ and R¹⁶ are, independently, hydrogen, halo, nitro, amino, unsubstituted C₁₋₄ alkyl, substituted C₁₋₄ alkyl, unsubstituted C₂₋₄ alkenyl, substituted C₂₋₄ alkenyl, unsubstituted C₂₋₄ alkynyl, substituted C₂₋₄ alkynyl, C₁₋₄ alkoxy wherein at least two (more preferably at least three) of the substituents R¹³, R¹⁴, R¹⁵ and R¹⁶ are H
A¹ is halo, unsubstituted aryl, substituted aryl, unsubstituted arylalkyl, substituted arylalkyl, unsubstituted arylalkynyl, substituted arylalkynyl, unsubstituted aryloxy, substituted aryloxy, unsubstituted arylalkyloxy, substituted arylalkyloxy, unsubstituted arylthio or substituted arylthio;
or a salt or a N-oxide thereof.

More particularly, compounds for use in the present invention are shown in Table 1 below. In Table 1 the free valencies are the point of attachment of the relevant substituent. Therefore the compound I.a 016 is the following compound (2-(6-phenyl-pyridin-2-yl)-quinazoline):

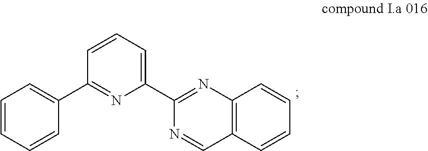

Likewise the compound I.a 001 is the following compound (2-(6-chloro-pyridin-2-yl)-quinazoline):

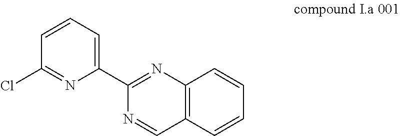

and the compound I.a 035 is the following compound (2-(5-trifluormethyl-6-phenylethynyl-pyridin-2-yl)-quinazoline):

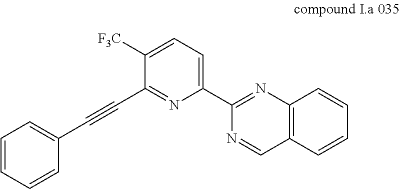

TABLE 1

| No. | A | R¹ |
|---|---|---|
| 001 | Cl | H |
| 002 | Cl | CH₃ |
| 003 | Cl | CH₂CH₃ |
| 004 | Cl | CF₃ |
| 005 | Cl | OCH₃ |
| 006 | Br | H |
| 007 | Br | CH₃ |
| 008 | Br | CH₂CH₃ |
| 009 | Br | CF₃ |
| 010 | Br | OCH₃ |
| 011 | I | H |
| 012 | I | CH₃ |
| 013 | I | CH₂CH₃ |
| 014 | I | CF₃ |
| 015 | I | OCH₃ |
| 016 | 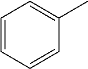 | H |
| 017 | 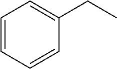 | H |
| 018 | 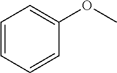 | H |
| 019 | 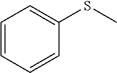 | H |
| 020 | 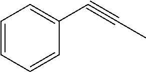 | H |
| 021 | 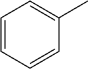 | CH₃ |
| 022 | 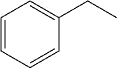 | CH₃ |
| 023 | 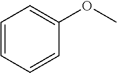 | CH₃ |
| 024 | 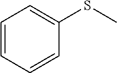 | CH₃ |
| 025 | 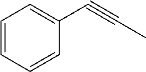 | CH₃ |
| 026 | 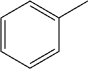 | CH₂CH₃ |
| 027 | 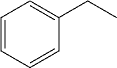 | CH₂CH₃ |
| 028 | 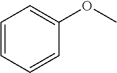 | CH₂CH₃ |
| 029 | 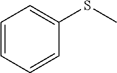 | CH₂CH₃ |
| 030 | 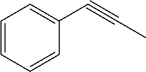 | CH₂CH₃ |
| 031 | 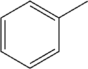 | CF₃ |
| 032 | 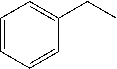 | CF₃ |
| 033 | 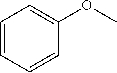 | CF₃ |
| 034 | 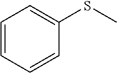 | CF₃ |
| 035 | 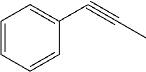 | CF₃ |
| 036 | 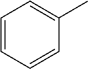 | OCH₃ |
| 037 | 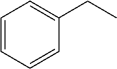 | OCH₃ |
| 038 | 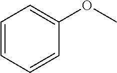 | OCH₃ |
| 039 | 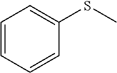 | OCH₃ |
| 040 | 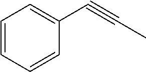 | OCH₃ |
| 041 | 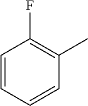 | H |
| 042 | 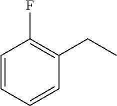 | H |
| 043 | 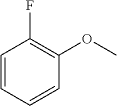 | H |
| 044 | 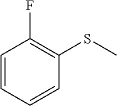 | H |
| 045 | 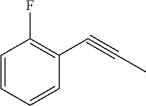 | H |
| 046 | 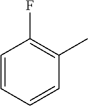 | CH₃ |
| 047 | 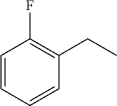 | CH₃ |
| 048 | 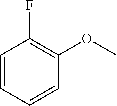 | CH₃ |
| 049 | 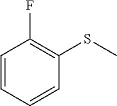 | CH₃ |
| 050 | 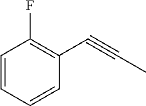 | CH₃ |
| 051 | 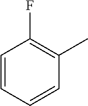 | CH₂CH₃ |
| 052 | 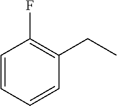 | CH₂CH₃ |
| 053 | 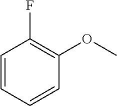 | CH₂CH₃ |
| 054 | 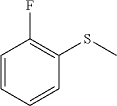 | CH₂CH₃ |
| 055 | 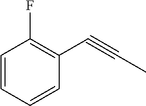 | CH₂CH₃ |
| 056 | 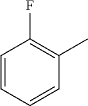 | CF₃ |
| 057 | 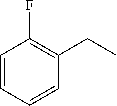 | CF₃ |
| 058 | 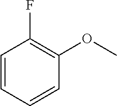 | CF₃ |
| 059 | 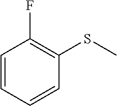 | CF₃ |
| 060 | 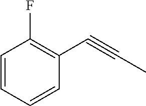 | CF₃ |
| 061 | 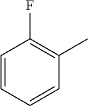 | OCH₃ |
| 062 | 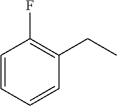 | OCH₃ |
| 063 | 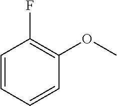 | OCH₃ |
| 064 | 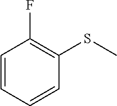 | OCH₃ |
| 065 | 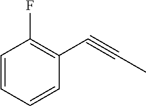 | OCH₃ |
| 066 | 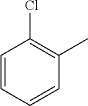 | H |
| 067 | 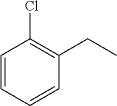 | H |
| 068 | 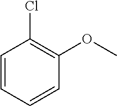 | H |
| 069 | 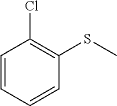 | H |
| 070 | 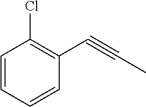 | H |
| 071 | 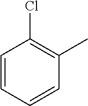 | CH₃ |
| 072 | 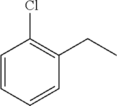 | CH₃ |
| 073 | 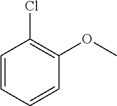 | CH₃ |
| 074 | 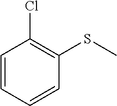 | CH₃ |
| 075 | 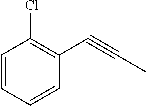 | CH₃ |
| 076 | 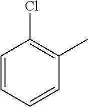 | CH₂CH₃ |
| 077 | 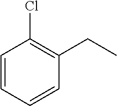 | CH₂CH₃ |
| 078 | 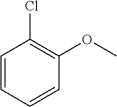 | CH₂CH₃ |
| 079 | 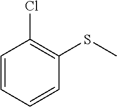 | CH₂CH₃ |
| 080 | 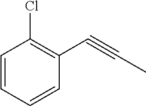 | CH₂CH₃ |
| 081 | 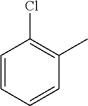 | CF₃ |
| 082 | 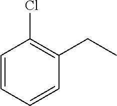 | CF₃ |
| 083 | 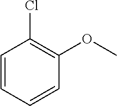 | CF₃ |
| 084 | 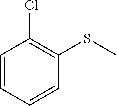 | CF₃ |
| 085 | 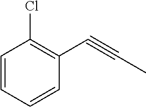 | CF₃ |
| 086 | 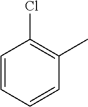 | OCH₃ |
| 087 | 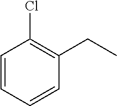 | OCH₃ |
| 088 | 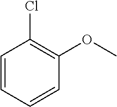 | OCH₃ |
| 089 | 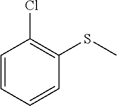 | OCH₃ |
| 090 | 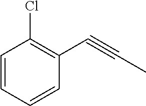 | OCH₃ |
| 091 | 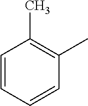 | H |
| 092 | 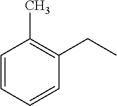 | H |
| 093 | 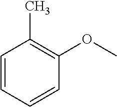 | H |
| 094 | 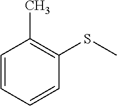 | H |
| 095 | 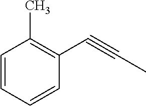 | H |
| 096 | 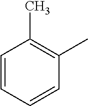 | CH₃ |
| 097 | 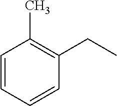 | CH₃ |
| 098 | 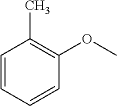 | CH₃ |
| 099 | 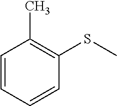 | CH₃ |
| 100 | 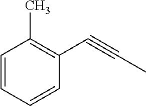 | CH₃ |
| 101 | 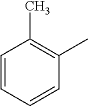 | CH₂CH₃ |
| 102 | 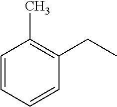 | CH₂CH₃ |
| 103 | 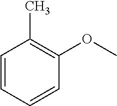 | CH₂CH₃ |
| 104 | 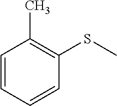 | CH₂CH₃ |
| 105 | 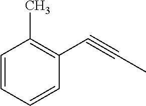 | CH₂CH₃ |
| 106 | 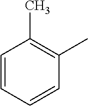 | CF₃ |
| 107 | 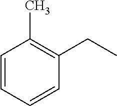 | CF₃ |
| 108 | 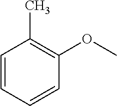 | CF₃ |
| 109 | 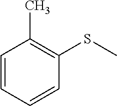 | CF₃ |
| 110 | 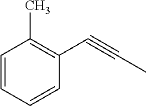 | CF₃ |
| 111 | 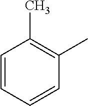 | OCH₃ |
| 112 | 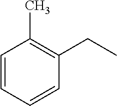 | OCH₃ |
| 113 | 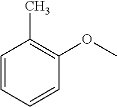 | OCH₃ |
| 114 | 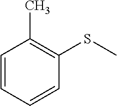 | OCH₃ |
| 115 | 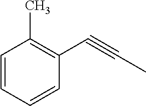 | OCH₃ |
| 116 | 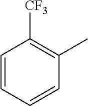 | H |
| 117 | 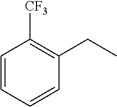 | H |
| 118 | 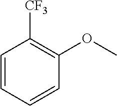 | H |
| 119 | 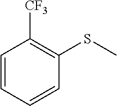 | H |
| 120 | 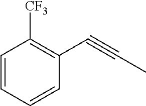 | H |
| 121 | 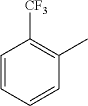 | CH₃ |
| 122 | 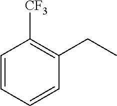 | CH₃ |
| 123 | 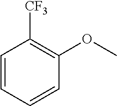 | CH₃ |
| 124 | 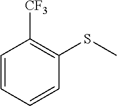 | CH₃ |
| 125 | 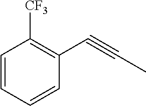 | CH₃ |
| 126 | 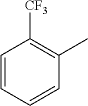 | CH₂CH₃ |
| 127 | 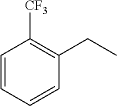 | CH₂CH₃ |
| 128 | 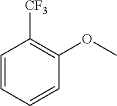 | CH₂CH₃ |
| 129 | 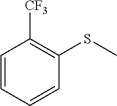 | CH₂CH₃ |
| 130 | 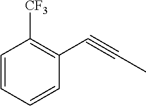 | CH₂CH₃ |
| 131 | 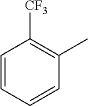 | CF₃ |
| 132 | 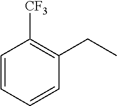 | CF₃ |
| 133 | 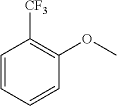 | CF₃ |
| 134 | 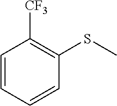 | CF₃ |
| 135 | 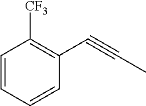 | CF₃ |
| 136 | 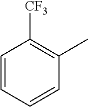 | OCH₃ |
| 137 | 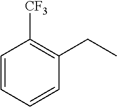 | OCH₃ |
| 138 | 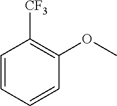 | OCH₃ |
| 139 | 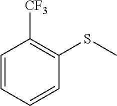 | OCH₃ |
| 140 | 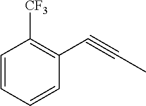 | OCH₃ |
| 141 | 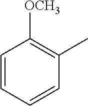 | H |
| 142 | 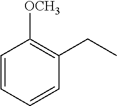 | H |
| 143 | 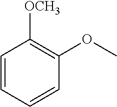 | H |
| 144 | 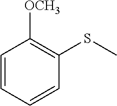 | H |
| 145 | 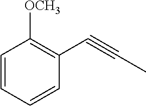 | H |
| 146 | 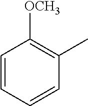 | CH₃ |
| 147 | 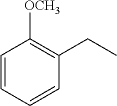 | CH₃ |
| 148 | 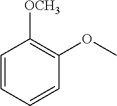 | CH₃ |
| 149 | 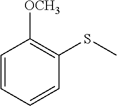 | CH₃ |
| 150 | 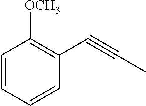 | CH₃ |
| 151 | 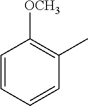 | CH₂CH₃ |
| 152 | 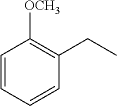 | CH₂CH₃ |
| 153 | 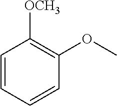 | CH₂CH₃ |
| 154 | 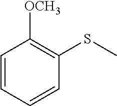 | CH₂CH₃ |
| 155 | 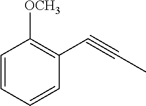 | CH₂CH₃ |
| 156 | 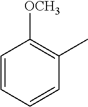 | CF₃ |
| 157 | 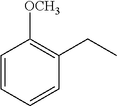 | CF₃ |
| 158 | 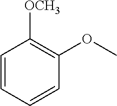 | CF₃ |
| 159 | 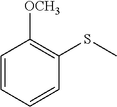 | CF₃ |
| 160 | 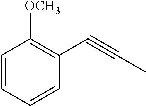 | CF₃ |
| 161 | 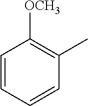 | OCH₃ |
| 162 | 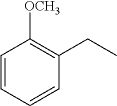 | OCH₃ |
| 163 | 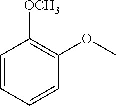 | OCH₃ |
| 164 | 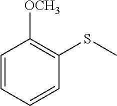 | OCH₃ |
| 165 | 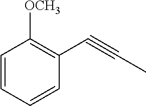 | OCH₃ |
| 166 | 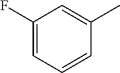 | H |
| 167 | 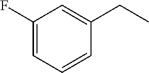 | H |
| 168 | 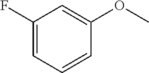 | H |
| 169 | 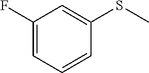 | H |
| 170 | 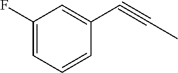 | H |
| 171 | 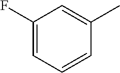 | CH₃ |
| 172 | 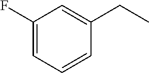 | CH₃ |
| 173 | 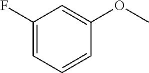 | CH₃ |
| 174 | 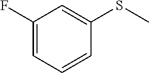 | CH₃ |
| 175 | 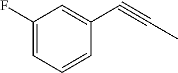 | CH₃ |
| 176 | 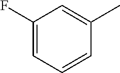 | CH₂CH₃ |
| 177 | 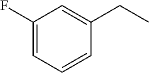 | CH₂CH₃ |
| 178 | 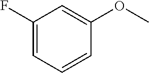 | CH₂CH₃ |
| 179 | 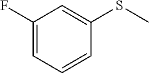 | CH₂CH₃ |
| 180 | 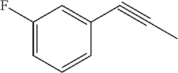 | CH₂CH₃ |
| 181 | 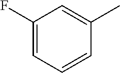 | CF₃ |
| 182 | 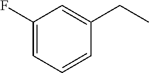 | CF₃ |
| 183 | 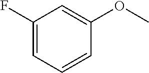 | CF₃ |
| 184 | 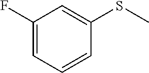 | CF₃ |
| 185 | 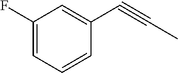 | CF₃ |
| 186 | 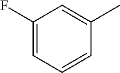 | OCH₃ |
| 187 | 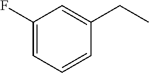 | OCH₃ |
| 188 | 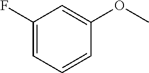 | OCH₃ |
| 189 | 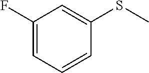 | OCH₃ |
| 190 | 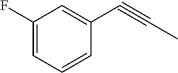 | OCH₃ |
| 191 | 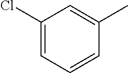 | H |
| 192 | 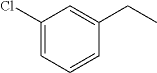 | H |
| 193 | 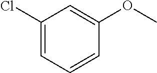 | H |
| 194 | 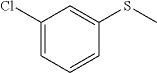 | H |
| 195 | 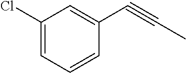 | H |
| 196 | 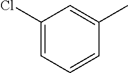 | CH₃ |
| 197 | 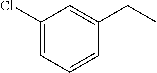 | CH₃ |
| 198 | 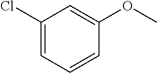 | CH₃ |
| 199 | 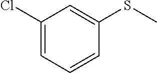 | CH₃ |
| 200 | 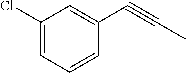 | CH₃ |
| 201 | 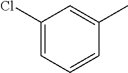 | CH₂CH₃ |
| 202 | 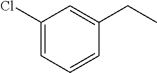 | CH₂CH₃ |
| 203 | 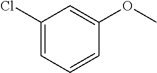 | CH₂CH₃ |
| 204 | 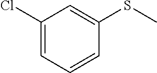 | CH₂CH₃ |
| 205 | 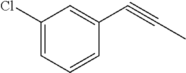 | CH₂CH₃ |
| 206 | 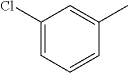 | CF₃ |
| 207 | 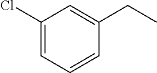 | CF₃ |
| 208 | 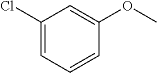 | CF₃ |
| 209 | 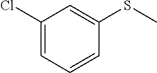 | CF₃ |
| 210 | 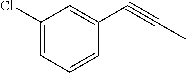 | CF₃ |
| 211 | 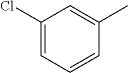 | OCH₃ |
| 212 | 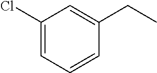 | OCH₃ |
| 213 | 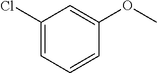 | OCH₃ |
| 214 | 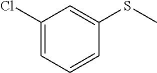 | OCH₃ |
| 215 | 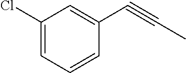 | OCH₃ |
| 216 | 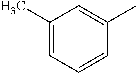 | H |
| 217 | 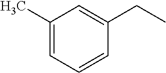 | H |
| 218 | 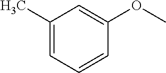 | H |
| 219 | 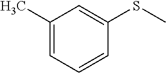 | H |
| 220 | 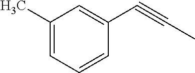 | H |
| 221 | 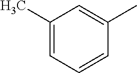 | CH₃ |
| 222 | 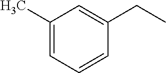 | CH₃ |
| 223 | 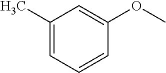 | CH₃ |
| 224 | 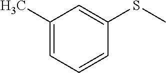 | CH₃ |
| 225 | 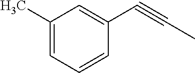 | CH₃ |
| 226 | 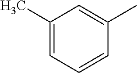 | CH₂CH₃ |
| 227 | 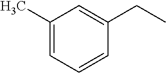 | CH₂CH₃ |
| 228 | 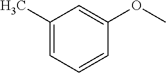 | CH₂CH₃ |
| 229 | 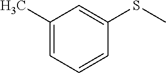 | CH₂CH₃ |
| 230 | 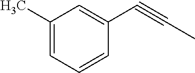 | CH₂CH₃ |
| 231 | 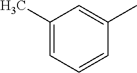 | CF₃ |
| 232 | 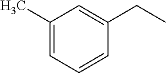 | CF₃ |
| 233 | 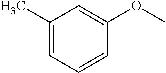 | CF₃ |
| 234 | 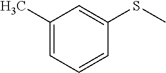 | CF₃ |
| 235 | 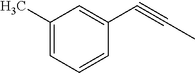 | CF₃ |
| 236 | 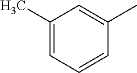 | OCH₃ |
| 237 | 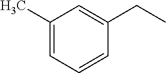 | OCH₃ |
| 238 | 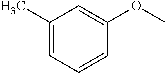 | OCH₃ |
| 239 | 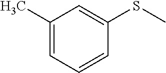 | OCH₃ |
| 240 | 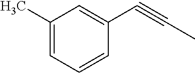 | OCH₃ |
| 241 | 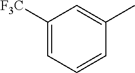 | H |
| 242 | 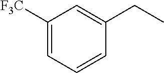 | H |
| 243 | 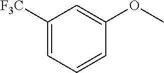 | H |
| 244 | 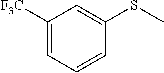 | H |
| 245 | 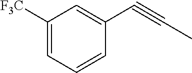 | H |
| 246 | 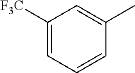 | CH₃ |
| 247 | 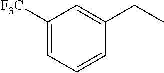 | CH₃ |
| 248 | 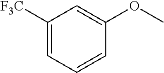 | CH₃ |
| 249 | 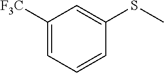 | CH₃ |
| 250 | 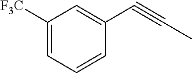 | CH₃ |
| 251 | 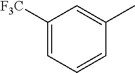 | CH₂CH₃ |
| 252 | 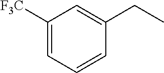 | CH₂CH₃ |
| 253 | 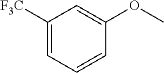 | CH₂CH₃ |
| 254 | 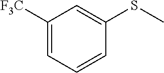 | CH₂CH₃ |
| 255 | 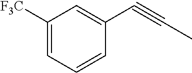 | CH₂CH₃ |
| 256 | 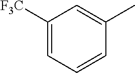 | CF₃ |
| 257 | 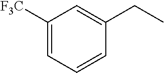 | CF₃ |
| 258 | 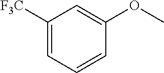 | CF₃ |
| 259 | 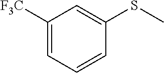 | CF₃ |
| 260 | 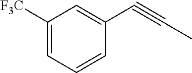 | CF₃ |
| 261 | 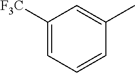 | OCH₃ |
| 262 | 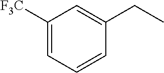 | OCH₃ |
| 263 | 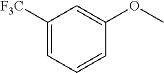 | OCH₃ |
| 264 | 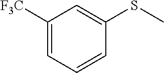 | OCH₃ |
| 265 | 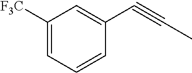 | OCH₃ |
| 266 | 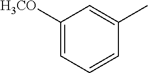 | H |
| 267 | 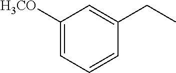 | H |
| 268 | 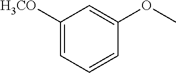 | H |
| 269 | 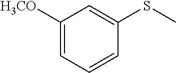 | H |
| 270 | 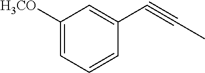 | H |
| 271 | 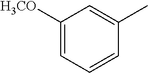 | CH₃ |
| 272 | 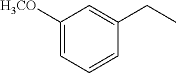 | CH₃ |
| 273 | 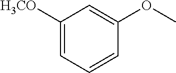 | CH₃ |
| 274 | 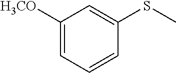 | CH₃ |
| 275 | 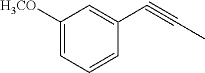 | CH₃ |
| 276 | 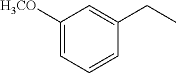 | CH₂CH₃ |
| 277 | 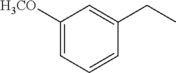 | CH₂CH₃ |
| 278 | 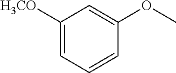 | CH₂CH₃ |
| 279 | 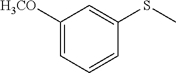 | CH₂CH₃ |
| 280 | 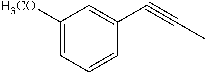 | CH₂CH₃ |
| 281 | 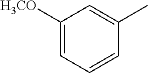 | CF₃ |
| 282 | 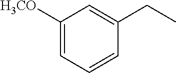 | CF₃ |
| 283 | 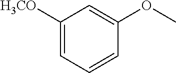 | CF₃ |
| 284 | 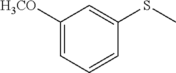 | CF₃ |
| 285 | 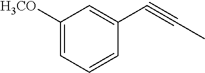 | CF₃ |
| 286 | 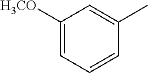 | OCH₃ |
| 287 | 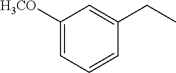 | OCH₃ |
| 288 | 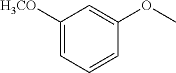 | OCH₃ |
| 289 | 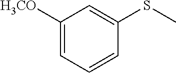 | OCH₃ |
| 290 | 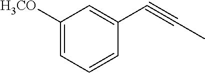 | OCH₃ |
| 291 | 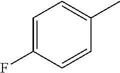 | H |
| 292 | 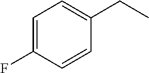 | H |
| 293 | 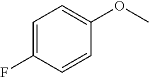 | H |
| 294 | 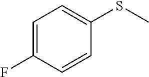 | H |
| 295 | 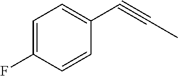 | H |
| 296 | 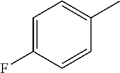 | CH₃ |
| 297 | 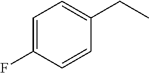 | CH₃ |
| 298 | 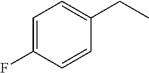 | CH₃ |
| 299 | 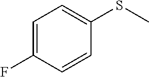 | CH₃ |
| 300 | 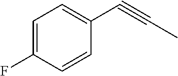 | CH₃ |
| 301 | 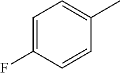 | CH₂CH₃ |
| 302 | 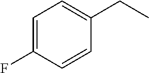 | CH₂CH₃ |
| 303 | 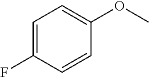 | CH₂CH₃ |
| 304 | 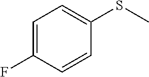 | CH₂CH₃ |
| 305 | 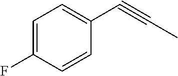 | CH₂CH₃ |
| 306 | 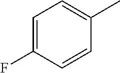 | CF₃ |
| 307 | 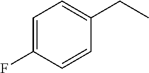 | CF₃ |
| 308 | 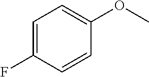 | CF₃ |
| 309 | 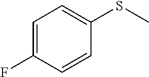 | CF₃ |
| 310 | 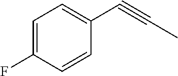 | CF₃ |
| 311 | 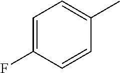 | OCH₃ |
| 312 | 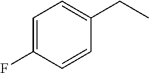 | OCH₃ |
| 313 | 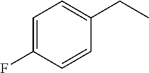 | OCH₃ |
| 314 | 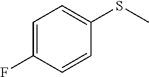 | OCH₃ |
| 315 | 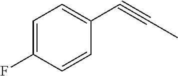 | OCH₃ |
| 316 | 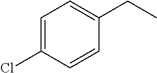 | H |
| 317 | 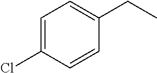 | H |
| 318 | 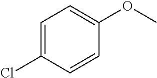 | H |
| 319 | 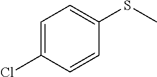 | H |
| 320 | 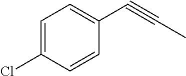 | H |
| 321 | 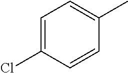 | CH₃ |
| 322 | 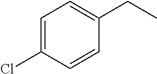 | CH₃ |
| 323 | 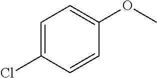 | CH₃ |
| 324 | 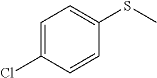 | CH₃ |
| 325 | 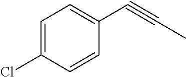 | CH₃ |
| 326 | 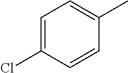 | CH₂CH₃ |
| 327 | 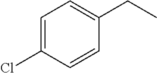 | CH₂CH₃ |
| 328 | 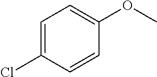 | CH₂CH₃ |
| 329 | 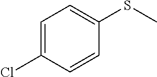 | CH₂CH₃ |
| 330 | 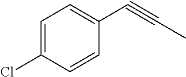 | CH₂CH₃ |
| 331 | 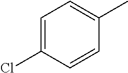 | CF₃ |
| 332 | 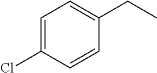 | CF₃ |
| 333 | 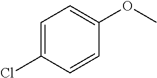 | CF₃ |
| 334 | 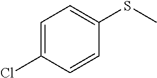 | CF₃ |
| 335 | 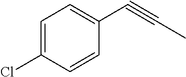 | CF₃ |
| 336 | 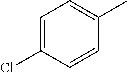 | OCH₃ |
| 337 | 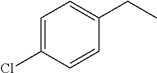 | OCH₃ |
| 338 | 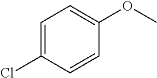 | OCH₃ |
| 339 | 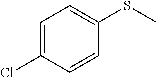 | OCH₃ |
| 340 | 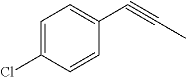 | OCH₃ |
| 341 | 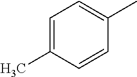 | H |
| 342 | 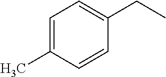 | H |
| 343 | 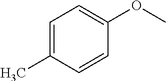 | H |
| 344 | 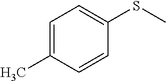 | H |
| 345 | 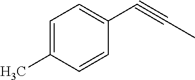 | H |
| 346 | 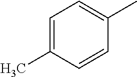 | CH₃ |
| 347 | 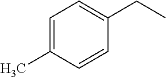 | CH₃ |
| 348 | 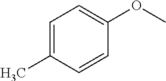 | CH₃ |
| 349 | 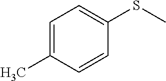 | CH₃ |
| 350 | 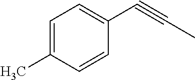 | CH₃ |
| 351 | 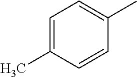 | CH₂CH₃ |
| 352 | 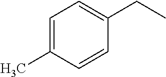 | CH₂CH₃ |
| 353 | 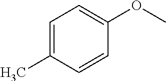 | CH₂CH₃ |
| 354 | 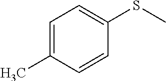 | CH₂CH₃ |
| 355 | 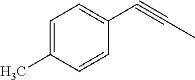 | CH₂CH₃ |
| 356 | 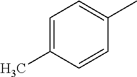 | CF₃ |
| 357 | 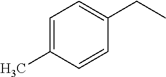 | CF₃ |
| 358 | 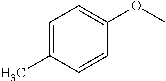 | CF₃ |
| 359 | 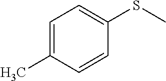 | CF₃ |
| 360 | 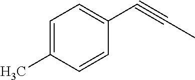 | CF₃ |
| 361 | 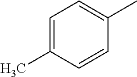 | OCH₃ |
| 362 | 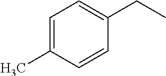 | OCH₃ |
| 363 | 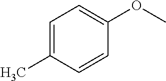 | OCH₃ |
| 364 | 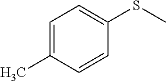 | OCH₃ |
| 365 | 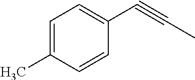 | OCH₃ |
| 366 | 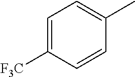 | H |
| 367 | 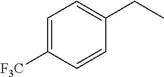 | H |
| 368 | 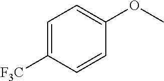 | H |
| 369 | 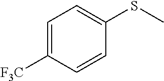 | H |
| 370 | 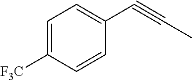 | H |
| 371 | 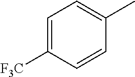 | CH₃ |
| 372 | 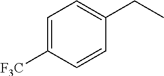 | CH₃ |
| 373 | 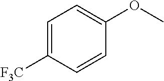 | CH₃ |
| 374 | 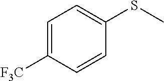 | CH₃ |
| 375 | 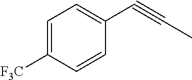 | CH₃ |
| 376 | 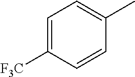 | CH₂CH₃ |
| 377 | 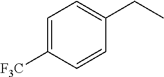 | CH₂CH₃ |
| 378 | 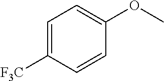 | CH₂CH₃ |
| 379 | 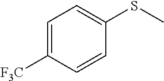 | CH₂CH₃ |
| 380 | 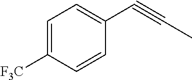 | CH₂CH₃ |
| 381 | 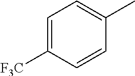 | CF₃ |
| 382 | 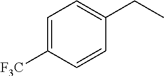 | CF₃ |
| 383 | 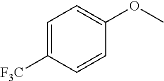 | CF₃ |
| 384 | 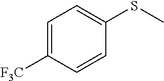 | CF₃ |
| 385 | 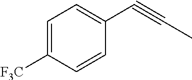 | CF₃ |
| 386 | 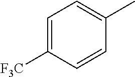 | OCH₃ |
| 387 | 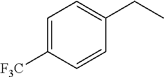 | OCH₃ |
| 388 | 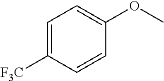 | OCH₃ |
| 389 | 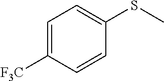 | OCH₃ |
| 390 | 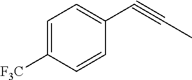 | OCH₃ |
| 391 | 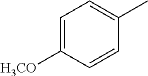 | H |
| 392 | 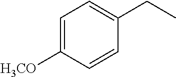 | H |
| 393 | 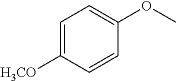 | H |
| 394 | 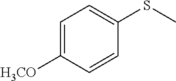 | H |
| 395 | 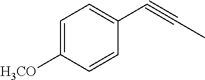 | H |
| 396 | 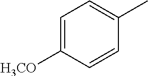 | CH₃ |
| 397 | 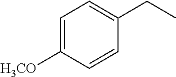 | CH₃ |
| 398 | 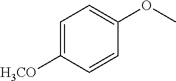 | CH₃ |
| 399 | 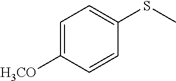 | CH₃ |
| 400 | 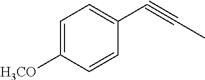 | CH₃ |
| 401 | 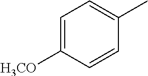 | CH₂CH₃ |
| 402 | 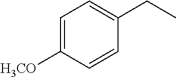 | CH₂CH₃ |
| 403 | 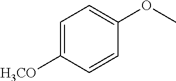 | CH₂CH₃ |
| 404 | 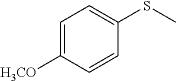 | CH₂CH₃ |
| 405 | 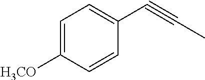 | CH₂CH₃ |
| 406 | 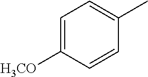 | CF₃ |
| 407 | 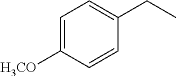 | CF₃ |
| 408 | 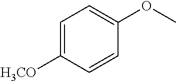 | CF₃ |
| 409 | 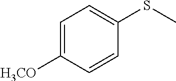 | CF₃ |
| 410 | 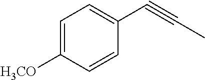 | CF₃ |
| 411 | 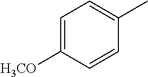 | OCH₃ |
| 412 | 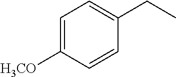 | OCH₃ |
| 413 | 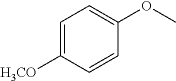 | OCH₃ |
| 414 | 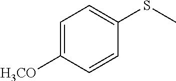 | OCH₃ |
| 415 | 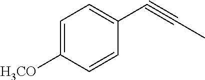 | OCH₃ |
| 416 | 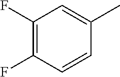 | H |
| 417 | 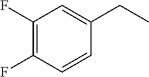 | H |
| 418 | 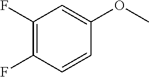 | H |
| 419 | 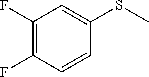 | H |
| 420 | 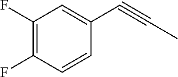 | H |
| 421 | 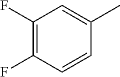 | CH₃ |
| 422 | 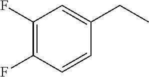 | CH₃ |
| 423 | 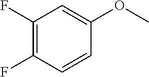 | CH₃ |
| 424 | 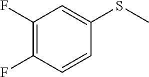 | CH₃ |
| 425 | 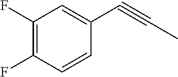 | CH₃ |
| 426 | 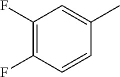 | CH₂CH₃ |
| 427 | 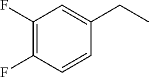 | CH₂CH₃ |
| 428 | 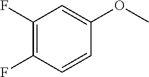 | CH₂CH₃ |
| 429 | 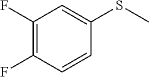 | CH₂CH₃ |
| 430 | 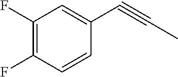 | CH₂CH₃ |
| 431 | 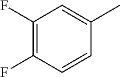 | CF₃ |
| 432 | 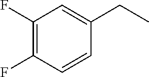 | CF₃ |
| 433 | 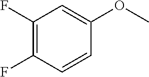 | CF₃ |
| 434 | 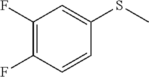 | CF₃ |
| 435 | 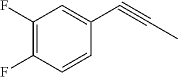 | CF₃ |
| 436 | 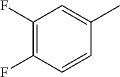 | OCH₃ |
| 437 | 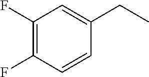 | OCH₃ |
| 438 | 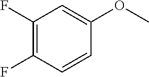 | OCH₃ |
| 439 | 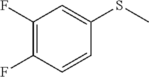 | OCH₃ |
| 440 | 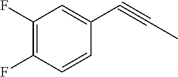 | OCH₃ |
| 441 | 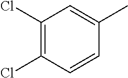 | H |
| 442 | 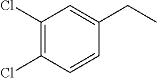 | H |
| 443 | 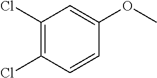 | H |
| 444 | 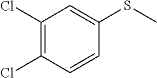 | H |
| 445 | 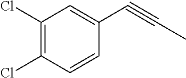 | H |
| 446 | 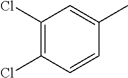 | CH₃ |
| 447 | 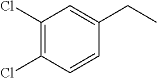 | CH₃ |
| 448 | 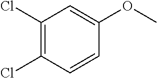 | CH₃ |
| 449 | 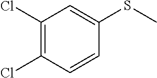 | CH₃ |
| 450 | 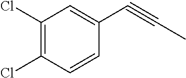 | CH₃ |
| 451 | 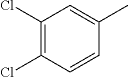 | CH₂CH₃ |
| 452 | 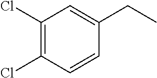 | CH₂CH₃ |
| 453 | 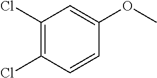 | CH₂CH₃ |
| 454 | 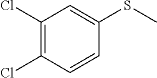 | CH₂CH₃ |
| 455 | 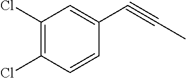 | CH₂CH₃ |
| 456 | 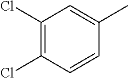 | CF₃ |
| 457 | 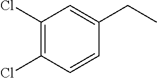 | CF₃ |
| 458 | 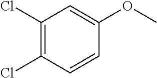 | CF₃ |
| 459 | 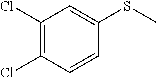 | CF₃ |
| 460 | 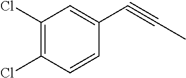 | CF₃ |
| 461 | 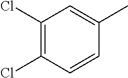 | OCH₃ |
| 462 | 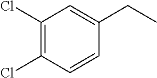 | OCH₃ |
| 463 | 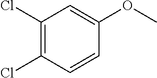 | OCH₃ |
| 464 | 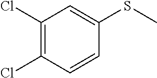 | OCH₃ |
| 465 | 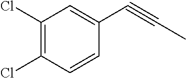 | OCH₃ |
| 466 | 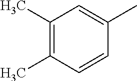 | H |
| 467 | 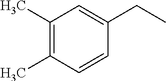 | H |
| 468 | 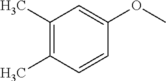 | H |
| 469 | 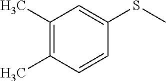 | H |
| 470 | 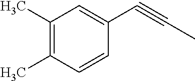 | H |
| 471 | 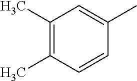 | CH₃ |
| 472 | 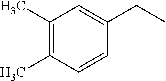 | CH₃ |
| 473 | 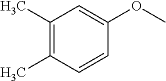 | CH₃ |
| 474 | 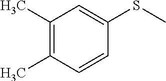 | CH₃ |
| 475 | 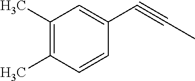 | CH₃ |
| 476 | 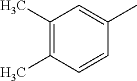 | CH₂CH₃ |
| 477 | 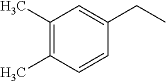 | CH₂CH₃ |
| 478 | 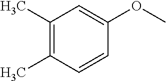 | CH₂CH₃ |
| 479 | 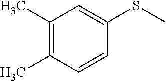 | CH₂CH₃ |
| 480 | 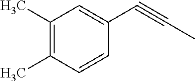 | CH₂CH₃ |
| 481 | 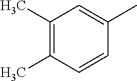 | CF₃ |
| 482 | 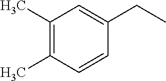 | CF₃ |
| 483 | 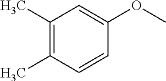 | CF₃ |
| 484 | 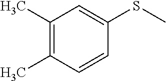 | CF₃ |
| 485 | 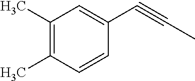 | CF₃ |
| 486 | 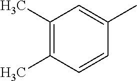 | OCH₃ |
| 487 | 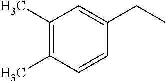 | OCH₃ |
| 488 | 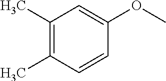 | OCH₃ |
| 489 | 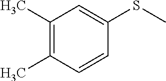 | OCH₃ |
| 490 | 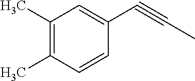 | OCH₃ |
| 491 | 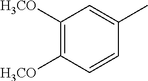 | H |
| 492 | 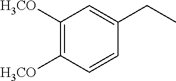 | H |
| 493 | 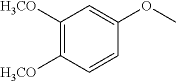 | H |
| 494 | 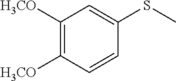 | H |
| 495 | 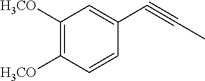 | H |
| 496 | 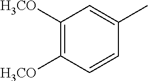 | CH₃ |
| 497 | 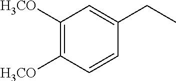 | CH₃ |
| 498 | 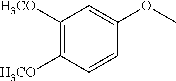 | CH₃ |
| 499 | 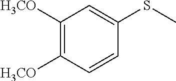 | CH₃ |
| 500 | 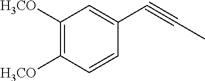 | CH₃ |
| 501 | 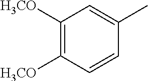 | CH₂CH₃ |
| 502 | 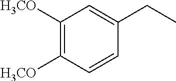 | CH₂CH₃ |
| 503 | 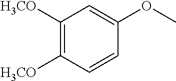 | CH₂CH₃ |
| 504 | 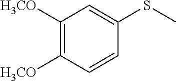 | CH₂CH₃ |
| 505 | 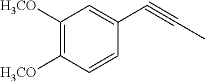 | CH₂CH₃ |
| 506 | 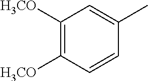 | CF₃ |
| 507 | 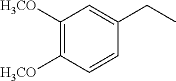 | CF₃ |
| 508 | 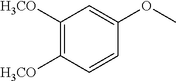 | CF₃ |
| 509 | 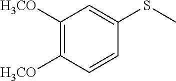 | CF₃ |
| 510 | 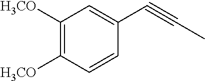 | CF₃ |
| 511 | 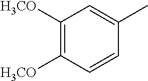 | OCH₃ |
| 512 | 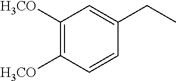 | OCH₃ |
| 513 | 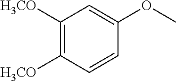 | OCH₃ |
| 514 | 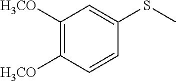 | OCH₃ |
| 515 | 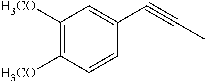 | OCH₃ |
| 516 | 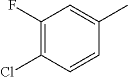 | H |
| 517 | 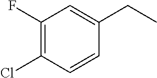 | H |
| 518 | 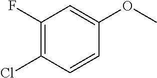 | H |
| 519 | 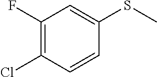 | H |
| 520 | 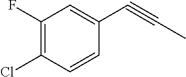 | H |
| 521 | 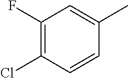 | CH₃ |
| 522 | 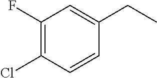 | CH₃ |
| 523 | 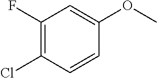 | CH₃ |
| 524 | 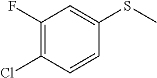 | CH₃ |
| 525 | 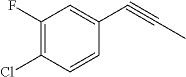 | CH₃ |
| 526 | 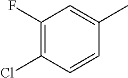 | CH₂CH₃ |
| 527 | 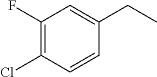 | CH₂CH₃ |
| 528 | 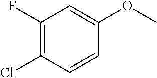 | CH₂CH₃ |
| 529 | 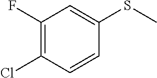 | CH₂CH₃ |
| 530 | 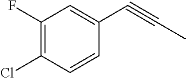 | CH₂CH₃ |
| 531 | 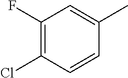 | CF₃ |
| 532 | 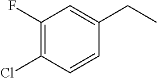 | CF₃ |
| 533 | 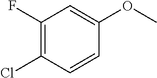 | CF₃ |
| 534 | 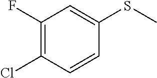 | CF₃ |
| 535 | 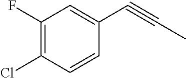 | CF₃ |
| 536 | 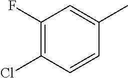 | OCH₃ |
| 537 | 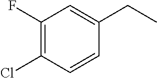 | OCH₃ |
| 538 | 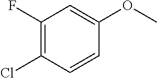 | OCH₃ |
| 539 | 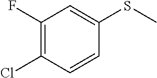 | OCH₃ |
| 540 | 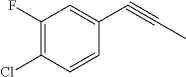 | OCH₃ |
| 541 | 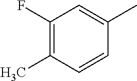 | H |
| 542 | 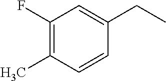 | H |
| 543 | 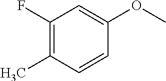 | H |
| 544 | 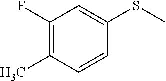 | H |
| 555 | 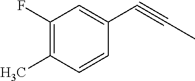 | H |
| 556 | 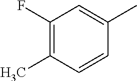 | CH₃ |
| 557 | 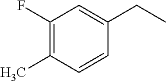 | CH₃ |
| 558 | 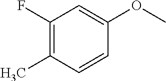 | CH₃ |
| 559 | 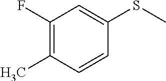 | CH₃ |
| 560 | 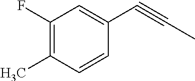 | CH₃ |
| 561 | 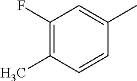 | CH₂CH₃ |
| 562 | 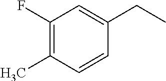 | CH₂CH₃ |
| 563 | 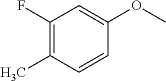 | CH₂CH₃ |
| 564 | 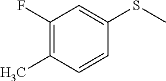 | CH₂CH₃ |
| 565 | 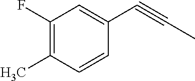 | CH₂CH₃ |
| 566 | 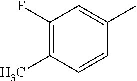 | CF₃ |
| 567 | 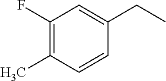 | CF₃ |
| 568 | 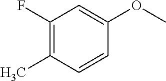 | CF₃ |
| 569 | 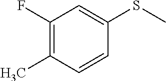 | CF₃ |
| 570 | 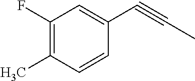 | CF₃ |
| 571 | 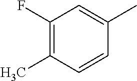 | OCH₃ |
| 572 | 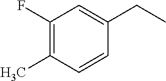 | OCH₃ |
| 573 | 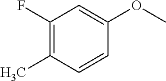 | OCH₃ |
| 574 | 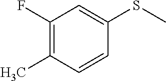 | OCH₃ |
| 575 | 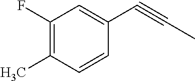 | OCH₃ |
| 576 | 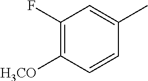 | H |
| 577 | 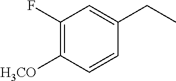 | H |
| 578 | 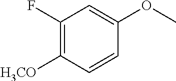 | H |
| 579 | 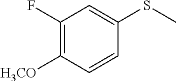 | H |
| 580 | 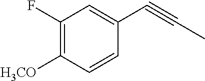 | H |
| 581 | 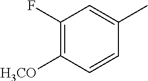 | CH₃ |
| 582 | 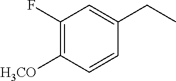 | CH₃ |
| 583 | 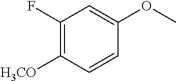 | CH₃ |
| 584 | 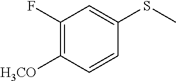 | CH₃ |
| 585 | 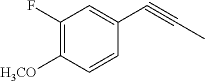 | CH₃ |
| 586 | 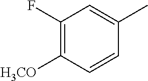 | CH₂CH₃ |
| 587 | 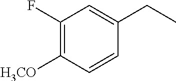 | CH₂CH₃ |
| 588 | 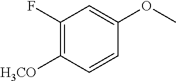 | CH₂CH₃ |
| 589 | 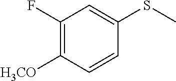 | CH₂CH₃ |
| 590 | 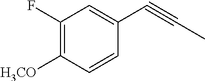 | CH₂CH₃ |
| 591 | 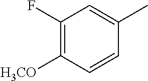 | CF₃ |
| 592 | 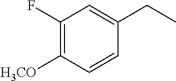 | CF₃ |
| 593 | 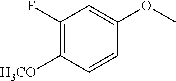 | CF₃ |
| 594 | 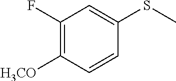 | CF₃ |
| 595 | 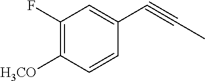 | CF₃ |
| 596 | 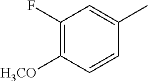 | OCH₃ |
| 597 | 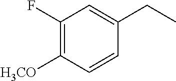 | OCH₃ |
| 598 | 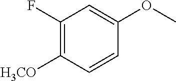 | OCH₃ |
| 599 | 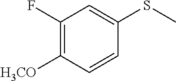 | OCH₃ |
| 600 | 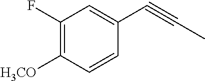 | OCH₃ |
| 601 | 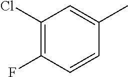 | H |
| 602 | 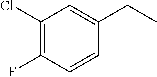 | H |
| 603 | 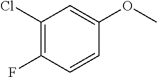 | H |
| 604 | 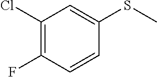 | H |
| 605 | 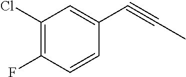 | H |
| 606 | 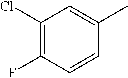 | CH₃ |
| 607 | 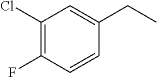 | CH₃ |
| 608 | 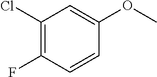 | CH₃ |
| 609 | 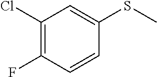 | CH₃ |
| 610 | 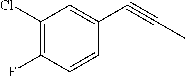 | CH₃ |
| 611 | 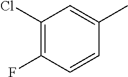 | CH₂CH₃ |
| 612 | 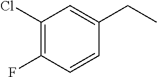 | CH₂CH₃ |
| 613 | 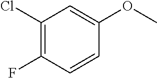 | CH₂CH₃ |
| 614 | 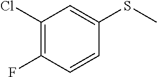 | CH₂CH₃ |
| 615 | 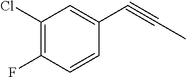 | CH₂CH₃ |
| 616 | 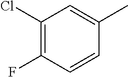 | CF₃ |
| 617 | 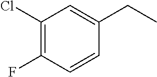 | CF₃ |
| 618 | 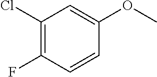 | CF₃ |
| 619 | 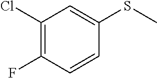 | CF₃ |
| 620 | 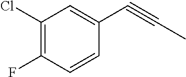 | CF₃ |
| 621 | 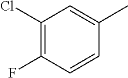 | OCH₃ |
| 622 | 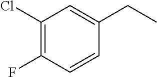 | OCH₃ |
| 623 | 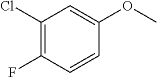 | OCH₃ |
| 624 | 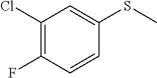 | OCH₃ |
| 625 | 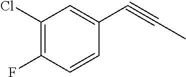 | OCH₃ |
| 626 | 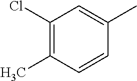 | H |
| 627 | 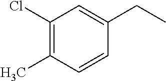 | H |
| 628 | 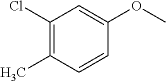 | H |
| 629 | 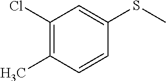 | H |
| 630 | 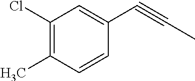 | H |
| 631 | 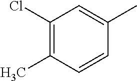 | CH₃ |
| 632 | 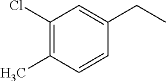 | CH₃ |
| 633 | 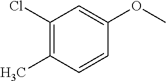 | CH₃ |
| 634 | 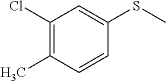 | CH₃ |
| 635 | 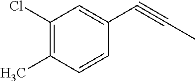 | CH₃ |
| 636 | 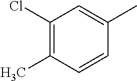 | CH₂CH₃ |
| 637 | 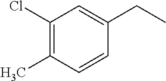 | CH₂CH₃ |
| 638 | 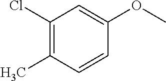 | CH₂CH₃ |
| 639 | 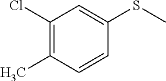 | CH₂CH₃ |
| 640 | 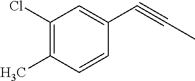 | CH₂CH₃ |
| 641 | 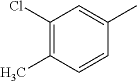 | CF₃ |
| 642 | 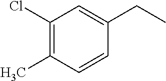 | CF₃ |
| 643 | 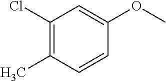 | CF₃ |
| 644 | 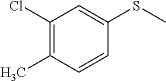 | CF₃ |
| 645 | 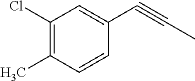 | CF₃ |
| 646 | 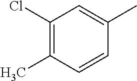 | OCH₃ |
| 647 | 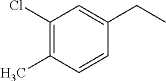 | OCH₃ |
| 648 | 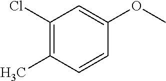 | OCH₃ |
| 649 | 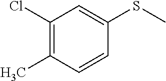 | OCH₃ |
| 650 | 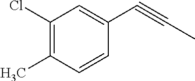 | OCH₃ |
| 651 | 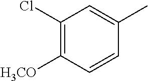 | H |
| 652 | 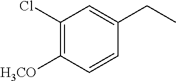 | H |
| 653 | 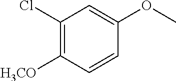 | H |
| 654 | 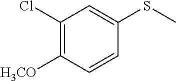 | H |
| 655 | 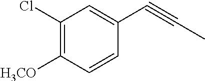 | H |
| 656 | 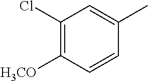 | CH₃ |
| 657 | 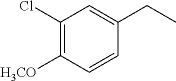 | CH₃ |
| 658 | 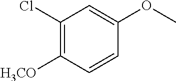 | CH₃ |
| 659 | 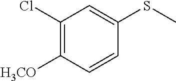 | CH₃ |
| 660 | 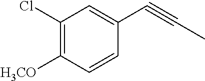 | CH₃ |
| 661 | 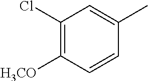 | CH₂CH₃ |
| 662 | 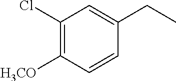 | CH₂CH₃ |
| 663 | 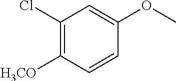 | CH₂CH₃ |
| 664 | 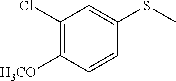 | CH₂CH₃ |
| 665 | 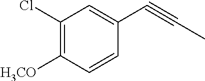 | CH₂CH₃ |
| 666 | 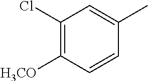 | CF₃ |
| 667 | 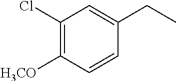 | CF₃ |
| 668 | 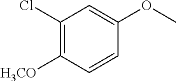 | CF₃ |
| 669 | 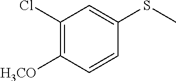 | CF₃ |
| 670 | 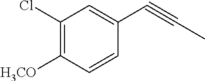 | CF₃ |
| 671 | 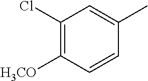 | OCH₃ |
| 672 | 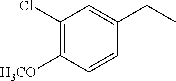 | OCH₃ |
| 673 | 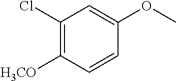 | OCH₃ |
| 674 | 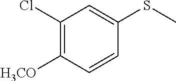 | OCH₃ |
| 675 | 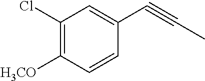 | OCH₃ |
| 676 | 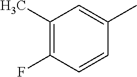 | H |
| 677 | 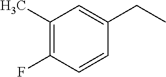 | H |
| 678 | 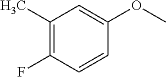 | H |
| 679 | 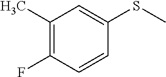 | H |
| 680 | 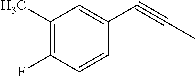 | H |
| 681 | 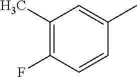 | CH₃ |
| 682 | 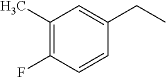 | CH₃ |
| 683 | 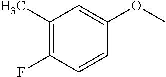 | CH₃ |
| 684 | 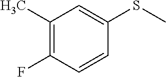 | CH₃ |
| 685 | 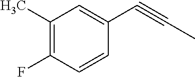 | CH₃ |
| 686 | 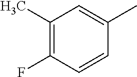 | CH₂CH₃ |
| 687 | 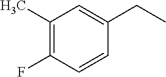 | CH₂CH₃ |
| 688 | 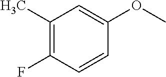 | CH₂CH₃ |
| 689 | 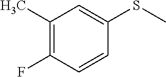 | CH₂CH₃ |
| 690 | 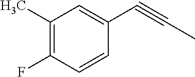 | CH₂CH₃ |
| 691 | 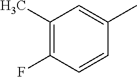 | CF₃ |
| 692 | 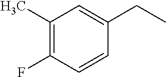 | CF₃ |
| 693 | 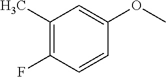 | CF₃ |
| 694 | 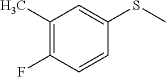 | CF₃ |
| 695 | 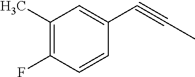 | CF₃ |
| 696 | 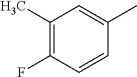 | OCH₃ |
| 697 | 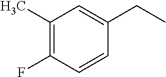 | OCH₃ |
| 698 | 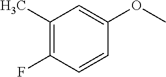 | OCH₃ |
| 699 | 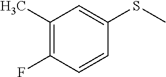 | OCH₃ |
| 700 | 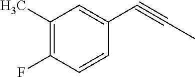 | OCH₃ |
| 701 | 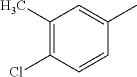 | H |
| 702 | 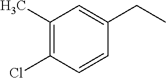 | H |
| 703 | 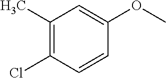 | H |
| 704 | 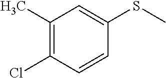 | H |
| 705 | 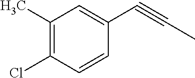 | H |
| 706 | 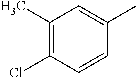 | CH₃ |
| 707 | 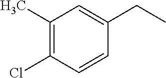 | CH₃ |
| 708 | 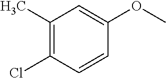 | CH₃ |
| 709 | 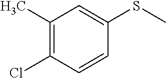 | CH₃ |
| 710 | 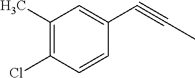 | CH₃ |
| 711 | 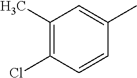 | CH₂CH₃ |
| 712 | 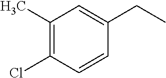 | CH₂CH₃ |
| 713 | 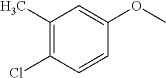 | CH₂CH₃ |
| 714 | 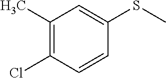 | CH₂CH₃ |
| 715 | 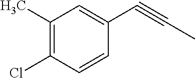 | CH₂CH₃ |
| 716 | 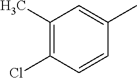 | CF₃ |
| 717 | 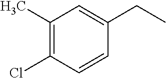 | CF₃ |
| 718 | 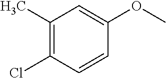 | CF₃ |
| 719 | 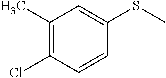 | CF₃ |
| 720 | 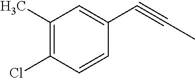 | CF₃ |
| 721 | 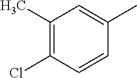 | OCH₃ |
| 722 | 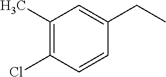 | OCH₃ |
| 723 | 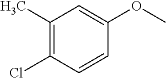 | OCH₃ |
| 724 | 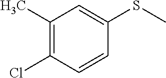 | OCH₃ |
| 725 | 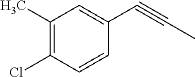 | OCH₃ |
| 726 | 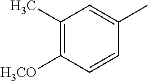 | H |
| 727 | 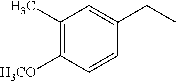 | H |
| 728 | 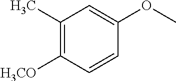 | H |
| 729 | 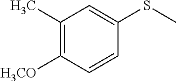 | H |
| 730 | 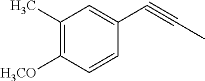 | H |
| 731 | 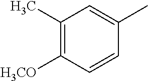 | CH₃ |
| 732 | 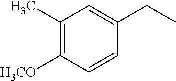 | CH₃ |
| 733 | 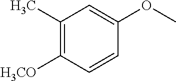 | CH₃ |
| 734 | 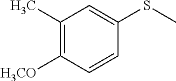 | CH₃ |
| 735 | 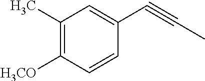 | CH₃ |
| 736 | 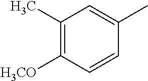 | CH₂CH₃ |
| 737 | 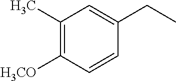 | CH₂CH₃ |
| 738 | 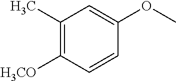 | CH₂CH₃ |
| 739 | 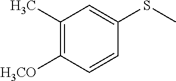 | CH₂CH₃ |
| 740 | 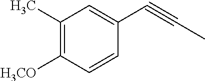 | CH₂CH₃ |
| 741 | 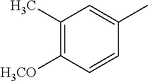 | CF₃ |
| 742 | 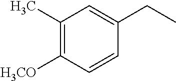 | CF₃ |
| 743 | 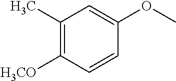 | CF₃ |
| 744 | 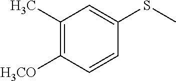 | CF₃ |
| 745 | 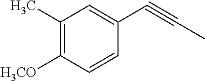 | CF₃ |
| 746 | 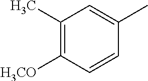 | OCH₃ |
| 747 | 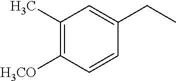 | OCH₃ |
| 748 | 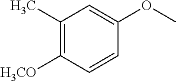 | OCH₃ |
| 749 | 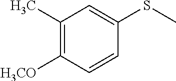 | OCH₃ |
| 750 | 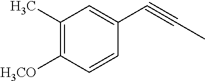 | OCH₃ |
| 751 | 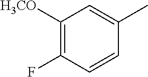 | H |
| 752 | 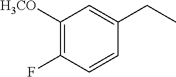 | H |
| 753 | 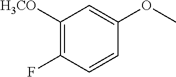 | H |
| 754 | 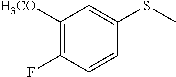 | H |
| 755 | 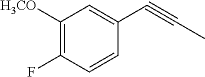 | H |
| 756 | 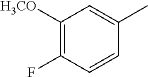 | CH₃ |
| 757 | 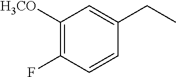 | CH₃ |
| 758 | 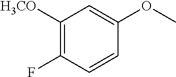 | CH₃ |
| 759 | 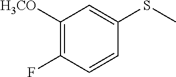 | CH₃ |
| 760 | 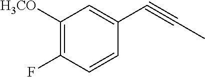 | CH₃ |
| 761 | 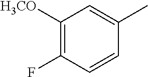 | CH₂CH₃ |
| 762 | 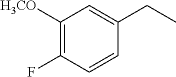 | CH₂CH₃ |
| 763 | 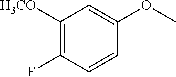 | CH₂CH₃ |
| 764 | 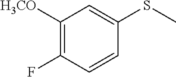 | CH₂CH₃ |
| 765 | 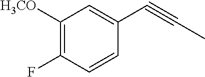 | CH₂CH₃ |
| 766 | 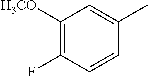 | CF₃ |
| 767 | 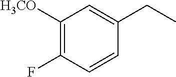 | CF₃ |
| 768 | 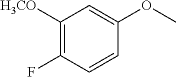 | CF₃ |
| 769 | 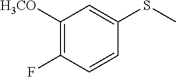 | CF₃ |
| 770 | 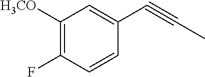 | CF₃ |
| 771 | 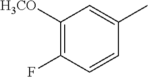 | OCH₃ |
| 772 | 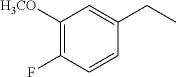 | OCH₃ |
| 773 | 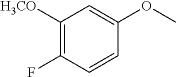 | OCH₃ |
| 774 | 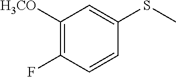 | OCH₃ |
| 775 | 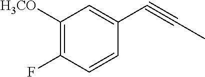 | OCH₃ |
| 776 | 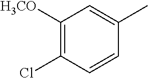 | H |
| 777 | 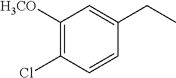 | H |
| 778 | 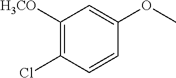 | H |
| 779 | 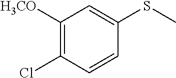 | H |
| 780 | 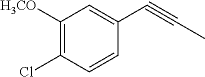 | H |
| 781 | 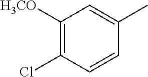 | CH₃ |
| 782 | 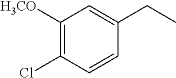 | CH₃ |
| 783 | 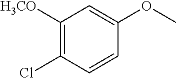 | CH₃ |
| 784 | 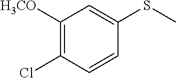 | CH₃ |
| 785 | 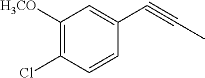 | CH₃ |
| 786 | 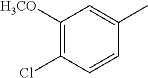 | CH₂CH₃ |
| 787 | 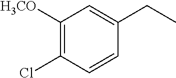 | CH₂CH₃ |
| 788 | 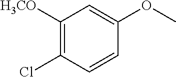 | CH₂CH₃ |
| 789 | 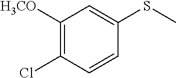 | CH₂CH₃ |
| 790 | 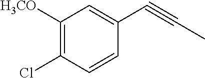 | CH₂CH₃ |
| 791 | 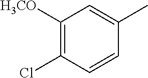 | CF₃ |
| 792 | 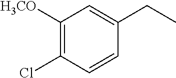 | CF₃ |
| 793 | 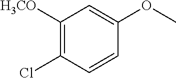 | CF₃ |
| 794 | 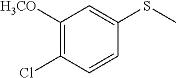 | CF₃ |
| 795 | 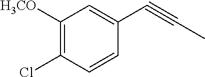 | CF₃ |
| 796 | 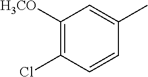 | OCH₃ |
| 797 | 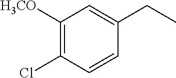 | OCH₃ |
| 798 | 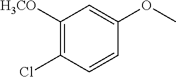 | OCH₃ |
| 799 | 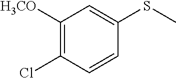 | OCH₃ |
| 800 | 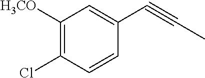 | OCH₃ |
| 801 | 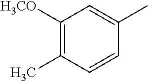 | H |
| 802 | 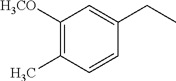 | H |
| 803 | 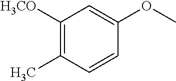 | H |
| 804 | 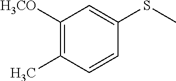 | H |
| 805 | 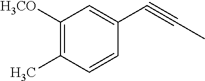 | H |
| 806 | 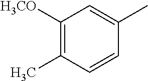 | CH₃ |
| 807 | 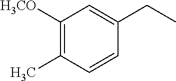 | CH₃ |
| 808 | 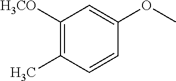 | CH₃ |
| 809 | 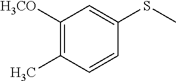 | CH₃ |
| 810 | 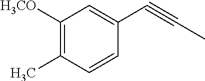 | CH₃ |
| 811 | 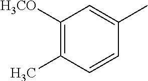 | CH₂CH₃ |
| 812 | 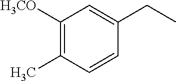 | CH₂CH₃ |
| 813 | 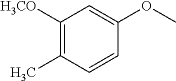 | CH₂CH₃ |
| 814 | 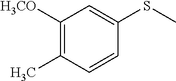 | CH₂CH₃ |
| 815 | 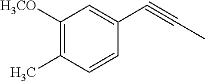 | CH₂CH₃ |
| 816 | 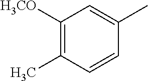 | CF₃ |
| 817 | 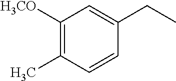 | CF₃ |
| 818 | 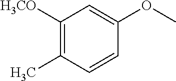 | CF₃ |
| 819 | 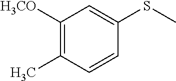 | CF₃ |
| 820 | 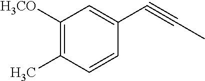 | CF₃ |
| 821 | 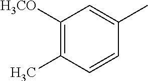 | OCH₃ |
| 822 | 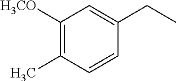 | OCH₃ |
| 823 | 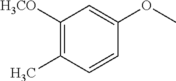 | OCH₃ |
| 824 | 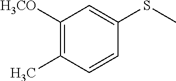 | OCH₃ |
| 825 | 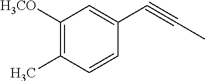 | OCH₃ |
| 826 | 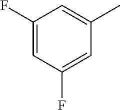 | H |
| 827 | 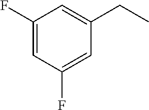 | H |
| 828 | 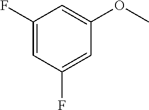 | H |
| 829 | 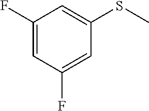 | H |
| 830 | 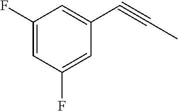 | H |
| 831 | 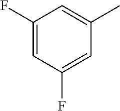 | CH₃ |
| 832 | 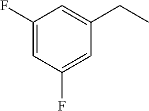 | CH₃ |
| 833 | 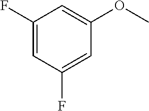 | CH₃ |
| 834 | 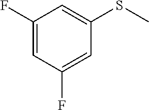 | CH₃ |
| 835 | 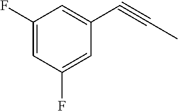 | CH₃ |
| 836 | 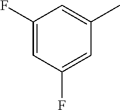 | CH₂CH₃ |
| 837 | 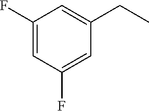 | CH₂CH₃ |
| 838 | 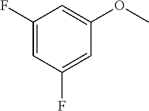 | CH₂CH₃ |
| 839 | 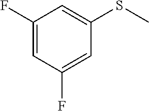 | CH₂CH₃ |
| 840 | 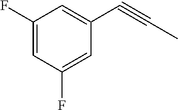 | CH₂CH₃ |
| 841 | 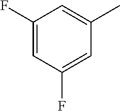 | CF₃ |
| 842 | 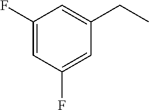 | CF₃ |
| 843 | 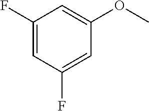 | CF₃ |
| 844 | 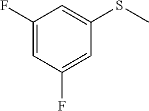 | CF₃ |
| 845 | 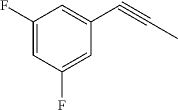 | CF₃ |
| 846 | 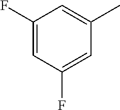 | OCH₃ |
| 847 | 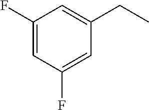 | OCH₃ |
| 848 | 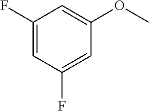 | OCH₃ |
| 849 | 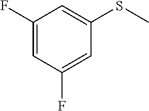 | OCH₃ |
| 850 | 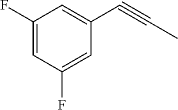 | OCH₃ |
| 851 | 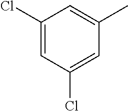 | H |
| 852 | 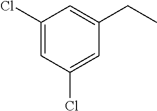 | H |
| 853 | 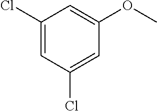 | H |
| 854 | 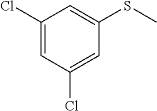 | H |
| 855 | 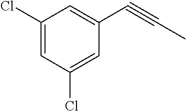 | H |
| 856 | 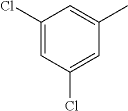 | CH₃ |
| 857 | 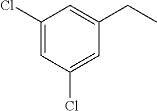 | CH₃ |
| 858 | 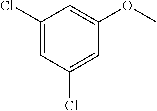 | CH₃ |
| 859 | 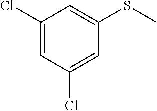 | CH₃ |
| 860 | 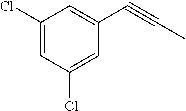 | CH₃ |
| 861 | 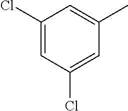 | CH₂CH₃ |
| 862 | 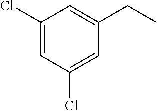 | CH₂CH₃ |
| 863 | 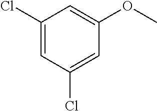 | CH₂CH₃ |
| 864 | 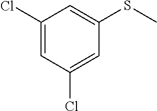 | CH₂CH₃ |
| 865 | 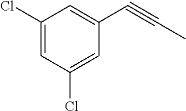 | CH₂CH₃ |
| 866 | 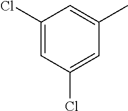 | CF₃ |
| 867 | 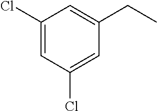 | CF₃ |
| 868 | 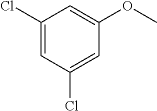 | CF₃ |
| 869 | 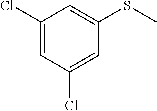 | CF₃ |
| 870 | 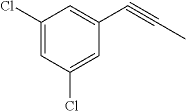 | CF₃ |
| 871 | 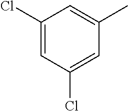 | OCH₃ |
| 872 | 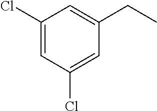 | OCH₃ |
| 873 | 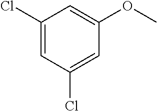 | OCH₃ |
| 874 | 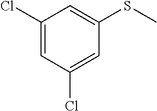 | OCH₃ |
| 875 | 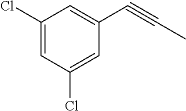 | OCH₃ |
| 876 | 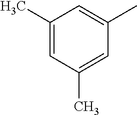 | H |
| 877 | 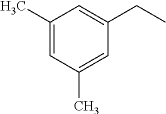 | H |
| 878 | 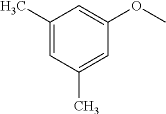 | H |
| 879 | 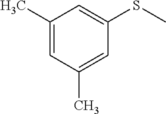 | H |
| 880 | 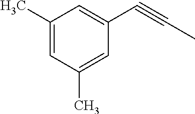 | H |
| 881 | 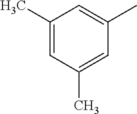 | CH₃ |
| 882 | 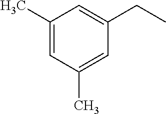 | CH₃ |
| 883 | 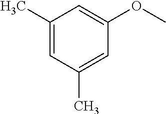 | CH₃ |
| 884 | 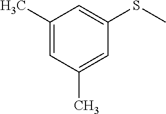 | CH₃ |
| 885 | 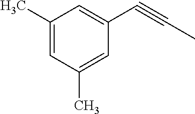 | CH₃ |
| 886 | 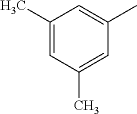 | CH₂CH₃ |
| 887 | 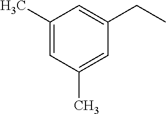 | CH₂CH₃ |
| 888 | 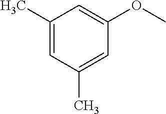 | CH₂CH₃ |
| 889 | 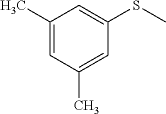 | CH₂CH₃ |
| 890 | 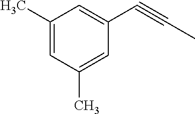 | CH₂CH₃ |
| 891 | 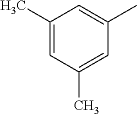 | CF₃ |
| 892 | 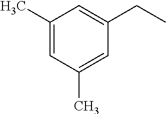 | CF₃ |
| 893 | 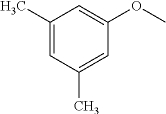 | CF₃ |
| 894 | 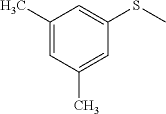 | CF₃ |
| 895 | 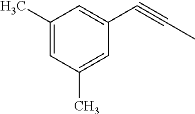 | CF₃ |
| 896 | 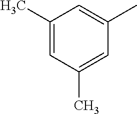 | OCH₃ |
| 897 | 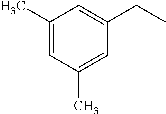 | OCH₃ |
| 898 | 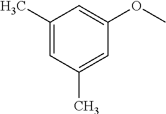 | OCH₃ |
| 899 | 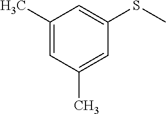 | OCH₃ |
| 900 | 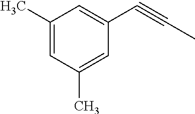 | OCH₃ |
| 901 | 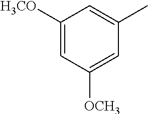 | H |
| 902 | 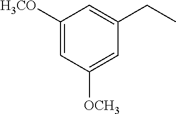 | H |
| 903 | 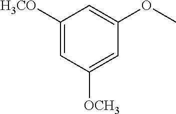 | H |
| 904 | 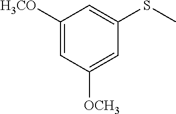 | H |
| 905 | 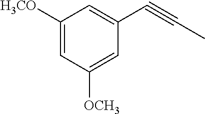 | H |
| 906 | 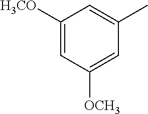 | CH₃ |
| 907 | 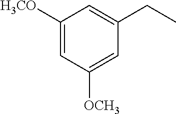 | CH₃ |
| 908 | 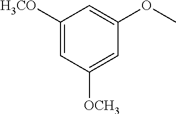 | CH₃ |
| 909 | 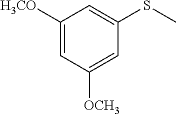 | CH₃ |
| 910 | 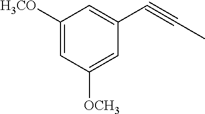 | CH₃ |
| 911 | 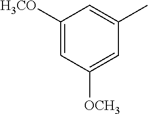 | CH₂CH₃ |
| 912 | 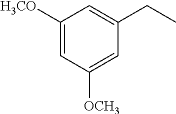 | CH₂CH₃ |
| 913 | 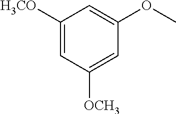 | CH₂CH₃ |
| 914 | 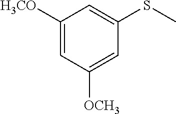 | CH₂CH₃ |
| 915 | 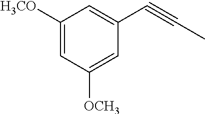 | CH₂CH₃ |
| 916 | 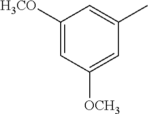 | CF₃ |
| 917 | 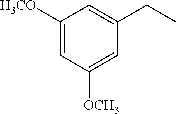 | CF₃ |
| 918 | 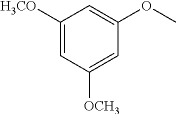 | CF₃ |
| 919 | 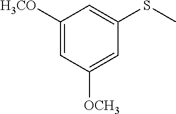 | CF₃ |
| 920 | 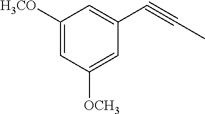 | CF₃ |
| 921 | 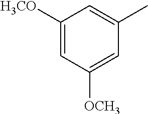 | OCH₃ |
| 922 | 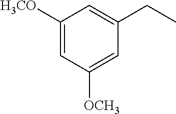 | OCH₃ |
| 923 | 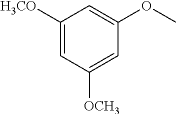 | OCH₃ |
| 924 | 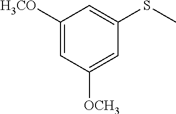 | OCH₃ |
| 925 | 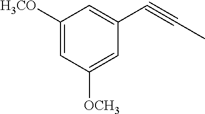 | OCH₃ |

wherein there are
a) 925 compounds of formula (I.a):

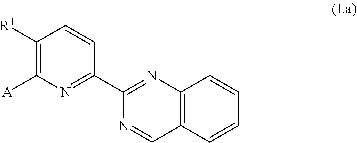

wherein R¹ and A are as defined in Table 1.
b) 925 compounds of formula (I.b):

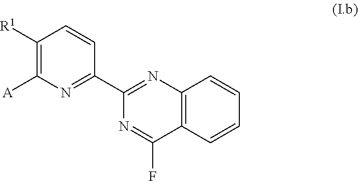

wherein R¹ and A are as defined in Table 1.
c) 925 compounds of formula (I.c):

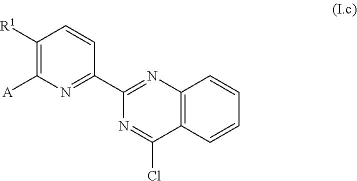

wherein R¹ and A are as defined in Table 1.
d) 925 compounds of formula (I.d):

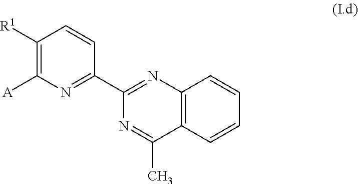

wherein R¹ and A are as defined in Table 1.
e) 925 compounds of formula (I.e):

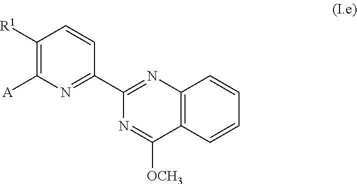

wherein R¹ and A are as defined in Table 1.
f) 925 compounds of formula (I.f):

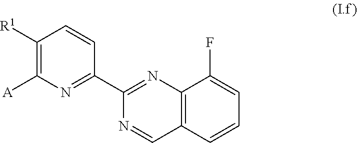

wherein R¹ and A are as defined in Table 1.
g) 925 compounds of formula (I.g):

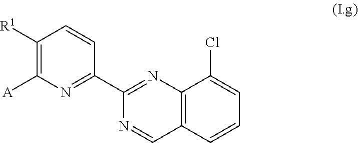

wherein R¹ and A are as defined in Table 1.
h) 925 compounds of formula (I.h):

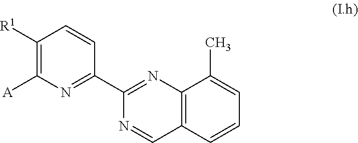

wherein R¹ and A are as defined in Table 1.
i) 925 compounds of formula (I.i):

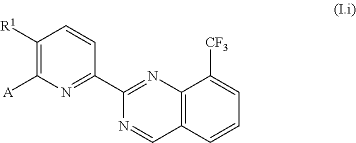

wherein R¹ and A are as defined in Table 1.
j) 925 compounds of formula (I.j):

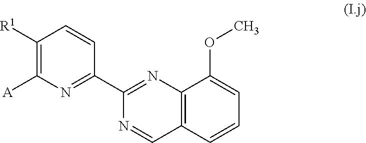

wherein R¹ and A are as defined in Table 1.
k) 925 compounds of formula (I.k):

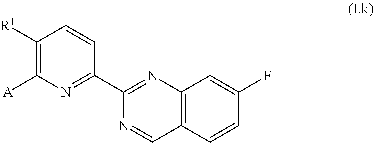

wherein R¹ and A are as defined in Table 1.
m) 925 compounds of formula (I.m):

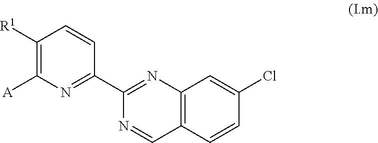

wherein R¹ and A are as defined in Table 1.
n) 925 compounds of formula (I.n):

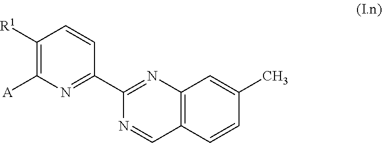

wherein R¹ and A are as defined in Table 1.
o) 925 compounds of formula (I.o):

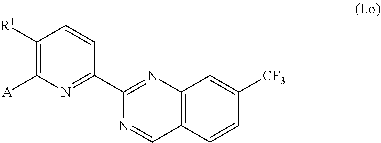

wherein R¹ and A are as defined in Table 1.
p) 925 compounds of formula (I.p):

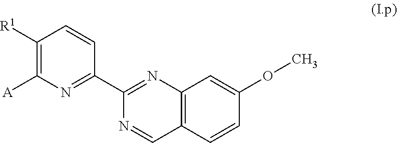

wherein R¹ and A are as defined in Table 1.
q) 925 compounds of formula (I.q):

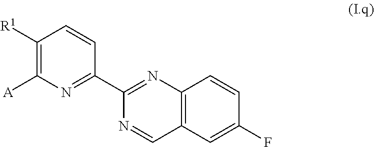

wherein R¹ and A are as defined in Table 1.
r) 925 compounds of formula (I.r):

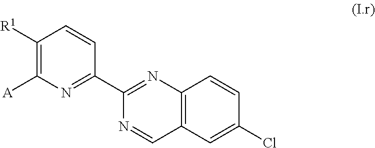

wherein R¹ and A are as defined in Table 1.
s) 925 compounds of formula (I.s):

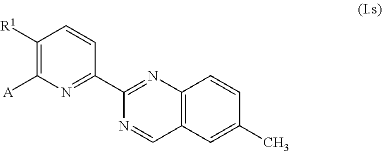

wherein R¹ and A are as defined in Table 1.
t) 925 compounds of formula (I.t):

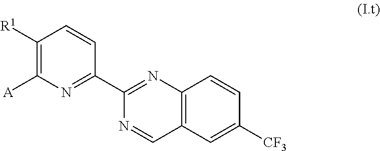

wherein R¹ and A are as defined in Table 1.
u) 925 compounds of formula (I.u):

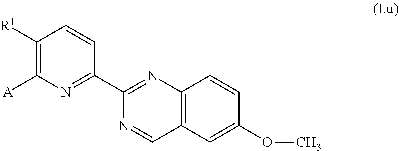

wherein R¹ and A are as defined in Table 1.
v) 925 compounds of formula (I.v):

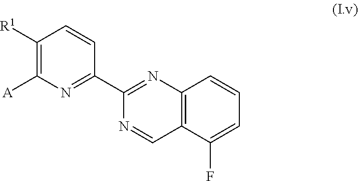

wherein R¹ and A are as defined in Table 1.
w) 925 compounds of formula (I.w):

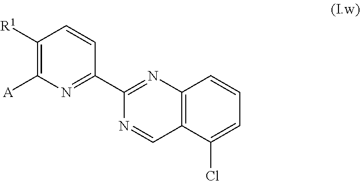

wherein R¹ and A are as defined in Table 1.
x) 925 compounds of formula (I.x):

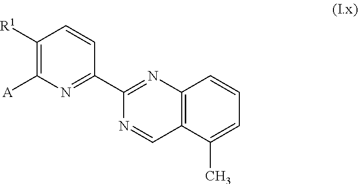

wherein R¹ and A are as defined in Table 1.
y) 925 compounds of formula (I.y):

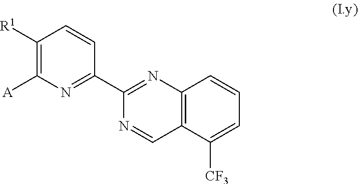

wherein R¹ and A are as defined in Table 1.
z) 925 compounds of formula (I.z):

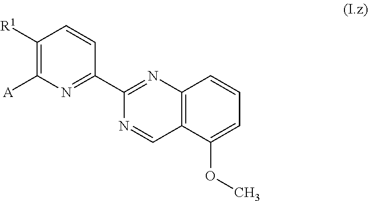

wherein R¹ and A are as defined in Table 1.

Preferred individual compounds are:
2-(5-methyl-6-o-tolylpyridin-2-yl)-quinazoline (Compound I.a 096);
2-[6-(4-fluoro-3-methylphenyl)-5-methylpyridin-2-yl]-quinazoline (Compound I.a 681),
2-[6-(3-fluoro-4-methoxy-phenyl)-5-methylpyridin-2-yl]-quinazoline (Compound I.a 581);
2-[6-(3,5-dimethylphenyl)-5-methylpyridin-2-yl]-quinazoline (Compound I.a 881);
2-[6-(3,5-difluorophenyl)-5-methylpyridin-2-yl]-quinazoline (Compound I.a 831);
2-[6-(3,4-difluorophenyl)-5-methylpyridin-2-yl]-quinazoline (Compound I.a 421);
6-Methyl-2-(5-methyl-6-phenylpyridin-2-yl)-quinazoline (Compound I.s 021);
2-[6-(2-chlorobenzyl)-pyridin-2-yl]-quinazoline (Compound I.a 067);
2-[6-(2-methylbenzyl)-pyridin-2-yl]-quinazoline (Compound I.a 092);
2-(6-benzyl-5-methylpyridin-2-yl)-quinazoline (Compound I.a 022);
2-(6-benzylpyridin-2-yl)-6-methylquinazoline (Compound I.s 017);
2-[6-(2,5-dimethyl-phenyl)-pyridin-2-yl]-quinazoline;
2-(6-benzyl-pyridin-2-yl)-4-methoxy-quinazoline;
2-[6-(2-fluoro-3-methyl-benzyl)-5-methyl-pyridin-2-yl]-quinazoline;
2-[6-(2-fluoro-3-methyl-benzyl)-pyridin-2-yl]-quinazoline;
4-methyl-2-(5-methyl-6-phenyl-pyridin-2-yl)-quinazoline;
2-[6-(4-methoxy-2-methyl-phenyl)-5-methyl-pyridin-2-yl]-quinazoline;
2-[6-(2-fluoro-5-methyl-phenyl)-5-methyl-pyridin-2-yl]-quinazoline;
2-[6-(4-fluoro-2-methyl-phenyl)-pyridin-2-yl]-quinazoline;
2-(6-cyclopropylethynyl-5-methyl-pyridin-2-yl)-quinazoline;
2-(6-phenoxy-pyridin-2-yl)-quinazoline;
2-(5-methyl-6-phenoxy-pyridin-2-yl)-quinazoline;
5-methyl-2-(5-methyl-6-phenyl-pyridin-2-yl)-quinazoline; and
2-[5-methoxy-6-(4-methoxy-phenyl)-pyridin-2-yl]-quinazoline.

Compounds of the invention and for use in the methods of the invention can be made, for example, by following the reaction schemes and the methods detailed below. The starting materials used for the preparation of the compounds of the invention may be purchased from usual commercial suppliers or may be prepared by known methods. The starting materials as well as the intermediates may be purified before use in the next step by state of the art methodologies such as chromatography, crystallization, distillation and filtration.

### Preparation of Compounds of Formula I

Compounds of formula (I) can be made as shown in the following schemes.

The compounds of formula I.1, wherein R¹, R³, R⁴, R⁵, R⁶ and A are as defined for formula I can be obtained by transformation of a compound of formula II, wherein R¹, R³, R⁴, R⁵, R⁶ and A are as defined for formula I with an oxidation agent, such as 2,3-dichloro-5,6-dicyano-p-benzoquinone, oxygen, manganese(IV) oxide or ammonium cerium(IV) nitrate.

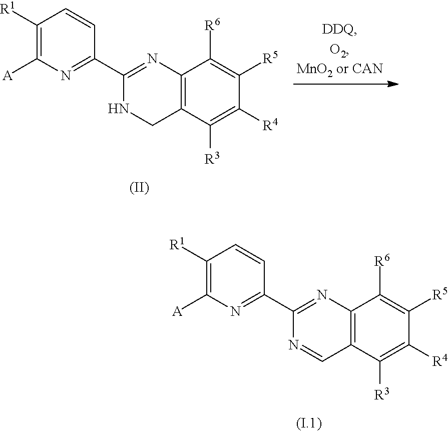

The compounds of formula I.1, wherein R¹, R³, R⁴, R⁵, R⁶ and A are as defined for formula I can be obtained by transformation of a compound of formula 1.2, wherein R¹, R³, R⁴, R⁵, R⁶ and A are as defined for formula I with a reducing agent such as Bu3SnH and a palladium catalyst.

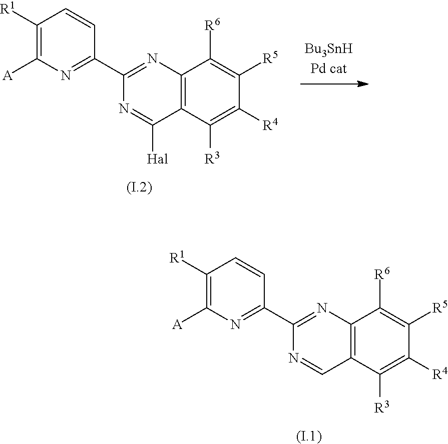

The compounds of formula II, wherein R¹, R³, R⁴, R⁵, R⁶ and A are as defined for formula I can be obtained by transformation of a compound of formula I.2, wherein R¹, R³, R⁴, R⁵, R⁶ and A are as defined for formula I and Hal is halogen, preferably chlorine or bromine, with a reduction agent such as hydrogen and a catalyst such as palladium on charcoal or raney-nickel, or with zinc and acetic acid.

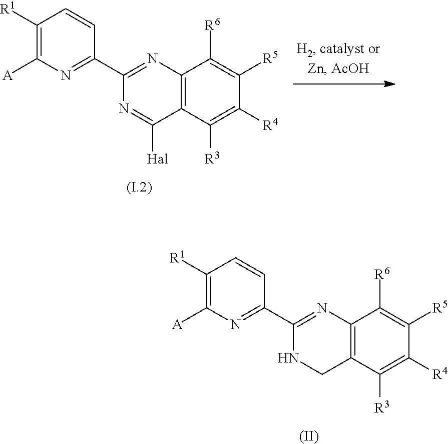

The compounds of formula I.2, wherein R¹, R³, R⁴, R⁵, R⁶ and A are as defined for formula I and Hal is halogen, preferably chlorine or bromine, can be obtained by transformation of a compound of formula III, wherein R¹, R³, R⁴, R⁵, R⁶ and A are as defined for formula I with a phosphorus oxyhalide, e.g. phosphorus oxychloride or phosphorus oxybromide, or a thionyl halide, e.g. thionyl chloride or thionyl bromide.

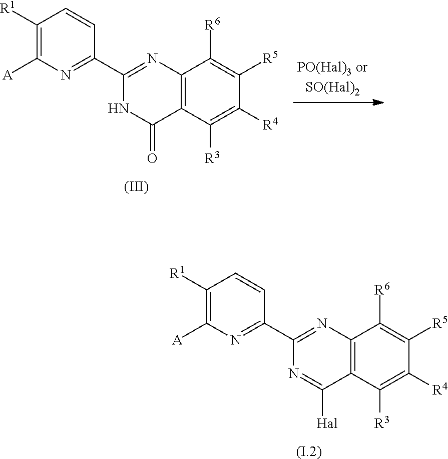

The compounds of formula III, wherein R¹, R³, R⁴, R⁵, R⁶ and A are as defined for formula I can be obtained by transformation of a compound of formula IV, wherein R¹ and A are as defined for formula I with an anthranilic acid of formula V, wherein R³, R⁴, R⁵ and R⁶ are as defined for formula I and a base, such as sodium hydride, sodium methylate, sodium ethylate or potassium methylate.

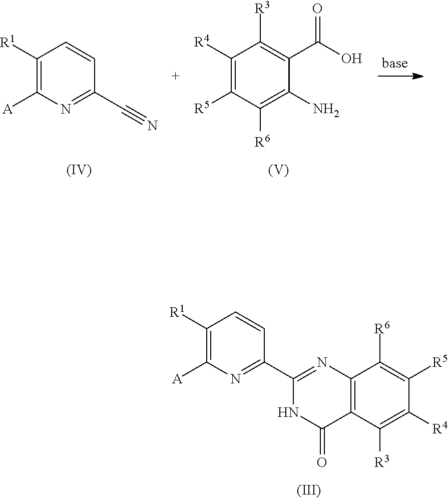

Alternatively, the compound of formula III wherein R¹, R³, R⁴, R⁵, R⁶ and A are as defined for formula I can be obtained by transformation of a compound of formula XII wherein R¹ and A are as defined for formula I and R⁷ is H with an anthranilic amide of formula Va, wherein R³, R⁴, R⁵ and R⁶ are as defined for formula I in a two-step procedure using a coupling reagent such as DCC, BOP or TBTU followed by treatment with a base such as NaOH in an alcoholic solvent. When R⁷ is C1-C6 alkyl the reaction can be performed in one step using a metal alcoholate in a alcoholic solvent.

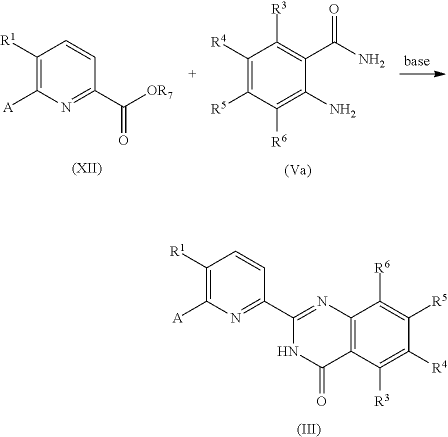

The anthranilic acid compounds of formula V are known compounds or may be obtained readily from known compounds using processes that are routine in the art and with which the skilled man will be familiar.

The compounds of formula IV, wherein R¹ and A are as defined for formula I can be obtained by transformation of a compound of formula VI, wherein R¹ and A are as defined for formula I with a cyanide, such as sodium cyanide, potassium cyanide or trimethylsilylcyanide and a base, such as triethylamine, ethyldiisopropylamine or pyridine.

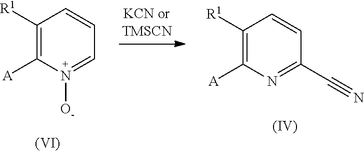

The compounds of formula VI, wherein R¹ and A are as defined for formula I can be obtained by transformation of a compound of formula VII, wherein R¹ and A are as defined for formula I with an oxidatizing agent, such as meta-chloroperbenzoic acid, hydrogen peroxide or oxone.

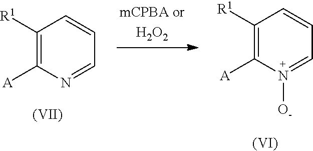

The mono- and disubstituted pyridines of formula VII are known compounds or may be obtained readily from known compounds using processes that are routine in the art and with which the skilled man will be familiar.

Alternatively, the compounds of formula I.1, wherein R¹, R³, R⁴, R⁵, R⁶ and A are as defined for formula I can be obtained by transformation of a compound of formula VIII, wherein R¹, R³, R⁴, R⁵, R⁶ and A are as defined for formula I with an oxidation agent, such as 2,3-dichloro-5,6-dicyano-p-benzoquinone, oxygen, manganese(IV) oxide or ammonium cerium(IV) nitrate.

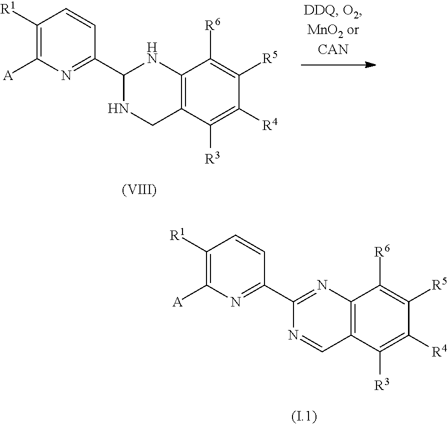

The compounds of formula VIII, wherein R¹, R³, R⁴, R⁵, R⁶ and A are as defined for formula I can be obtained by transformation of a compound of formula IX, wherein R¹ and A are as defined for formula I with a compound of formula X, wherein R³, R⁴, R⁵ and R⁶ are as defined for formula I, and thionyl chloride and a base, such as triethylamine, ethyldiisopropylamine or pyridine.

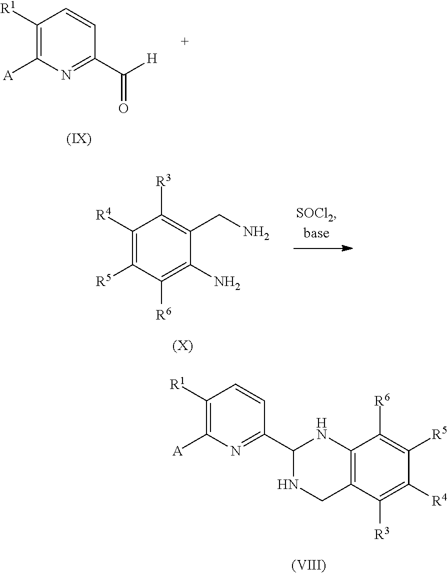

The 2-aminobenzylamines of formula X are known compounds or may be obtained readily from known compounds using processes that are routine in the art and with which the skilled man will be familiar.

The compounds of formula IX, wherein R¹ and A are as defined for formula I can be obtained by transformation of a compound of formula XI, wherein R¹ and A are as defined for formula I with N,N′-dicyclohexylcarbodiimide, dimethylsulfoxide and an acid, such as phosphoric acid, hydrochloric acid or sulfuric acid, or with manganese dioxide or 2,3-dichloro-5,6-dicyano-p-benzoquinone.

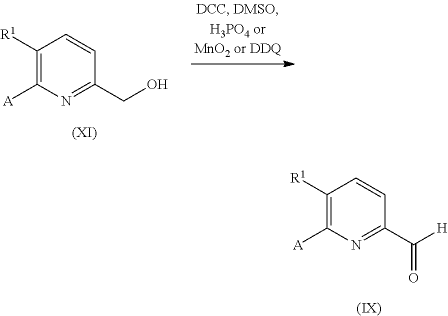

The compounds of formula XI, wherein R¹ and A are as defined for formula I can be obtained by transformation of a compound of formula XII, wherein R¹ and A are as defined for formula I and R⁷ is hydrogen or C₁-C₆alkyl, with an reducing agent, such as sodium borohydride, lithium aluminium hydride, lithium borohydride or diisobutylaluminum hydride.

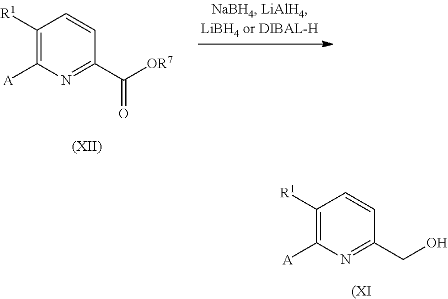

The compounds of formula XII, wherein R¹ and A are as defined for formula I and R⁷ is hydrogen or C₁-C₆alkyl, can be obtained by transformation of a compound of formula IV, wherein R¹ and A are as defined for formula I with a base, such as sodium methoxide, sodium ethoxide, potassium methoxide or potassium ethoxide in an alcohol and subsequent treatment with an acid, such as hydrochloric acid or sulfuric acid.

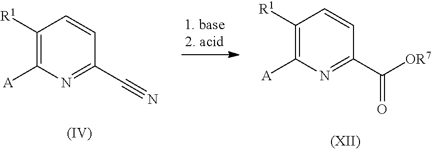

Alternatively the compounds of formula I.1, wherein R¹, R³, R⁴, R⁵, R⁶ and A are as defined for formula I can be obtained by transformation of a compound of formula XIII, wherein R¹ and A are as defined for formula I, or a salt of it, with a benzaldehyde of formula XIV, wherein R³, R⁴, R⁵ and R⁶ are as defined for formula I and R⁸ is a halogen, such as fluoro, chloro or bromo, or an amino group and a base, such as sodium carbonate, sodium bicarbonate or potassium carbonate.

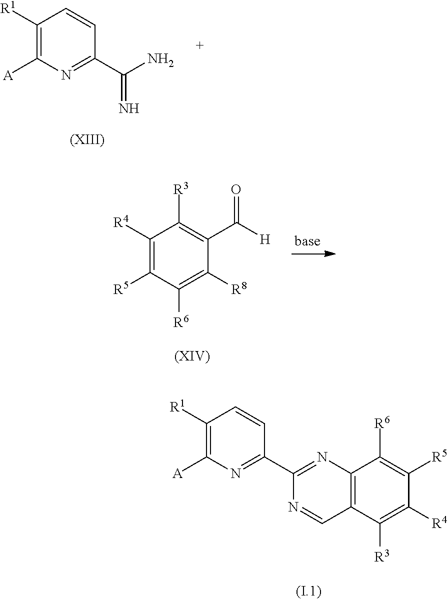

The 2-halobenzaldehydes of formula XIV are known compounds or may be obtained readily from known compounds using processes that are routine in the art and with which the skilled man will be familiar.

The compounds of formula XIII, wherein R¹ and A are as defined for formula I can be obtained by transformation of a compound of formula IV, wherein R¹ and A are as defined for formula I with ammonia.

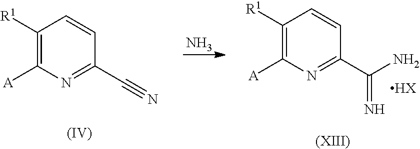

The compounds of formula I.3, wherein R¹, R³, R⁴, R⁵, R⁶ and A are as defined for formula I and R¹¹ is C₁₋₈ alkyl can be obtained by reaction of a compound of formula I.2, wherein R¹, R³, R⁴, R⁵, R⁶ and A are as defined for formula I and Hal is halogen, preferably chlorine or bromine, with an alcohol R¹¹—OH and a base, such as sodium hydride, potassium hydride, sodium carbonate, potassium carbonate, sodium hydroxide or potassium hydroxide.

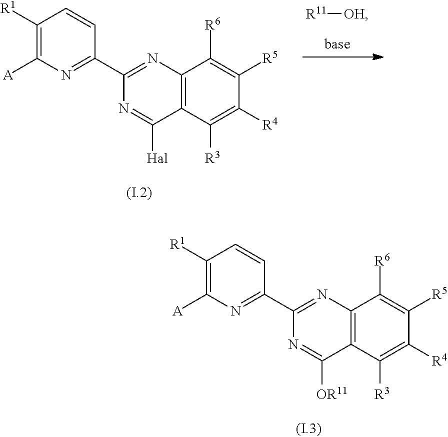

The compounds of formula I.4, wherein R¹, R³, R⁴, R⁵, R⁶ and A are as defined for formula I and R¹¹ is C₁₋₈ alkyl can be obtained by alkylation of a compound of formula I.2, wherein R¹, R³, R⁴, R⁵, R⁶ and A are as defined for formula I and Hal is halogen, preferably chlorine or bromine, with an organometallic species, such as methylmagnesium chloride, methylmagnesium bromide, trimethylaluminum or R¹¹B(OR⁷)₂ or trimethylboroxine.

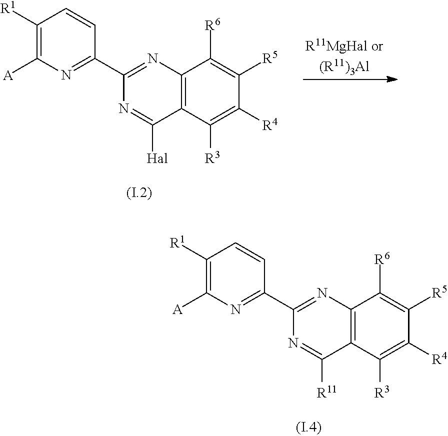

As an alternative The compounds of formula (IIw), wherein R¹, R³, R⁴, R⁵, R⁶ and A are as defined for formula I and R¹¹ is C₁₋₈ alkyl can be obtained by alkylation of a compound of formula I.1, wherein R¹, R³, R⁴, R⁵, R⁶ and A are as defined for formula I, with an organometallic species, such as methylmagnesium chloride, methylmagnesium bromide or alkyllithium.

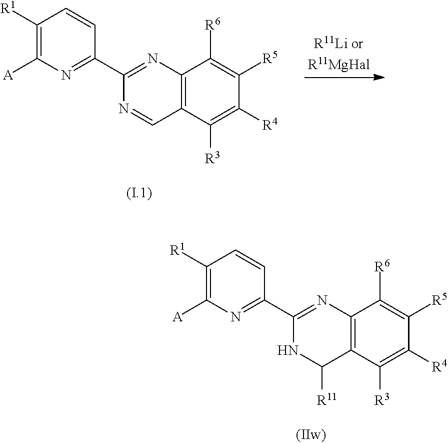

The compounds of formula I.4, wherein R¹, R³, R⁴, R⁵, R⁶ and A are as defined for formula I and R¹¹ is C₁₋₈ alkyl can be obtained by transformation of a compound of formula IIw, wherein R¹, R³, R⁴, R⁵, R⁶ and A are as defined for formula I and R¹¹ is C₁₋₈ alkyl with an oxidating agent, such as 2,3-dichloro-5,6-dicyano-p-benzoquinone, oxygen, manganese(IV) oxide or ammonium cerium(IV) nitrate.

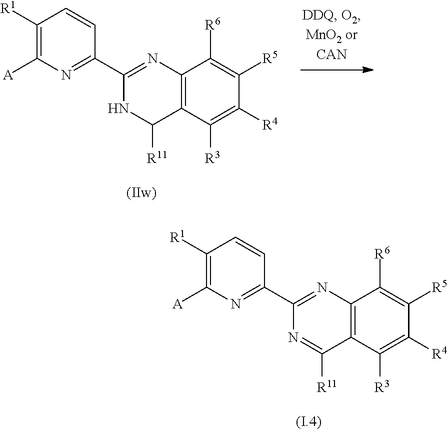

Alternatively the compounds of formula I, wherein R¹, R², R³, R⁴, R⁵, R⁶ and A are as defined for formula I can be obtained by transformation of a compound of formula XV, wherein R¹ and A are as defined for formula I and R⁹ is InR⁷₂, MgCl, MgBr, ZnCl, ZnBr, SnR⁷₃ or B(OR⁷)₂ with a compound of formula XVI, wherein R², R³, R⁴, R⁵ and R⁶ are as defined for formula I, R⁷ is hydrogen or C₁-C₆alkyl and R¹⁰ is a halogen, preferably chloro, bromo or iodo or a sulfonic ester such as a mesylate or tosylate and a catalyst, such as tetrakistriphenylphosphine, palladium dichloride, [1,1-bis(diphenylphosphino)ferrocene]dichloropalladium(II), palladium acetate or bis(diphenylphosphine)palladium(II) chloride.

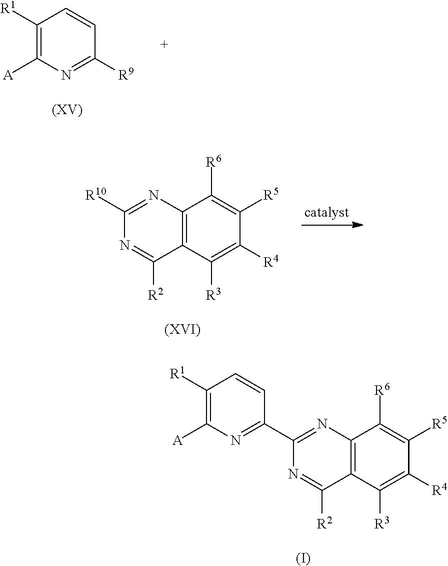

The metallo-substituted pyridines of formula XV and the 2-haloquinazolines of formula XVI are known compounds or may be obtained readily from known compounds using processes that are routine in the art and with which the skilled man will be familiar.

Alternatively the compounds of formula I, wherein R¹, R², R³, R⁴, R⁵, R⁶ and A are as defined for formula I can be obtained by transformation of a compound of formula XVII, wherein R¹ and A are as defined for formula I and R¹⁰ is a halogen, preferably chloro, bromo or iodo or a sulfonic ester such as a mesylate or tosylate with a compound of formula XVIII, wherein R², R³, R⁴, R⁵ and R⁶ are as defined for formula I, R⁹ is In, MgCl, MgBr, ZnCl, ZnBr, SnR⁷₃ or B(OR⁷)₂ and R⁷ is hydrogen or C₁-C₈alkyl and a catalyst, such as tetrakistriphenylphosphine, palladium dichloride, [1,1-bis(diphenylphosphino)ferrocene]dichloropalladium(II), palladium acetate or bis(diphenylphosphine)palladium(II) chloride.

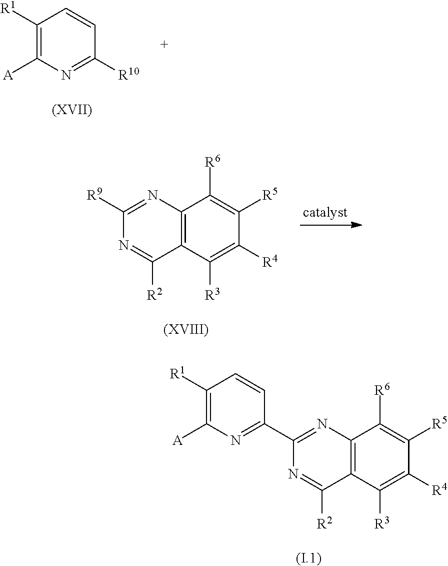

The di- and tri-substituted pyridines of formula XVII and the 2-metallo-substituted quinazolines of formula XVIII are known compounds or may be obtained readily from known compounds using processes that are routine in the art and with which the skilled man will be familiar.

The compounds of the present invention are useful in preventing microbial infection (in particular, fungal infection) or controlling plant pathogenic microbes (in particular, fungi) when they are applied to a plant or plant propagation material or the locus thereof in a microbicidally (fungicidally) effective amount. Accordingly, therefore, the present invention also provides a method of preventing and/or controlling microbial (fungal) infection in plants and/or plant propagation material comprising applying to the plant or plant propagation material or the locus thereof a microbicidally (fungicidally) effective amount of a compound of formula I. the present invention also provides a method of preventing and/or controlling microbial (fungal) infection in plants and/or plant propagation material comprising applying to the plant or plant propagation material or the locus thereof a microbicidally (fungicidally) effective amount of a compound of formula I and/or

By ‘preventing’ or ‘controlling’ is meant reducing the infestation of microbes (fungus) to such a level that an improvement is demonstrated.

By ‘plant propagation material’ is meant generative parts of a plant including seeds of all kinds (fruit, tubers, bulbs, grains etc), roots, rhizomes, cuttings, cut shoots and the like. Plant propagation material may also include plants and young plants which are to be transplanted after germination or after emergence from the soil.

By ‘locus’ is meant the place (e.g. the field) on which the plants to be treated are growing, or where the seeds of cultivated plants are sown, or the place where the seed will be placed into the soil.

The compounds of the present invention may be used against phytopathogenic fungi of the following classes: Fungi imperfecti (e.g. Alternaria spp.), Basidiomycetes (e.g. Corticium spp., Ceratobasidium spp., Waitea spp., Thanatephorus spp., Rhizoctonia spp., Hemileia spp., Puccinia spp., Phakopsora spp., Ustilago spp., Tilletia spp.), Ascomycetes (e.g. Venturia spp., Blumeria spp., Erysiphe spp., Podosphaera spp., Uncinula spp., Monilinia spp., Sclerotinia spp., Colletotrichum spp., Glomerella spp., Fusarium spp., Gibberella spp., Monographella spp., Phaeosphaeria spp., Mycosphaerella spp., Cercospora spp., Pyrenophora spp., Rhynchosporium spp., Magnaporthe spp., Gaeumannomyces spp., Oculimacula spp., Ramularia spp., Botryotinia spp.) and Oomycetes (e.g. Phytophthora spp., Pythium spp., Plasmopara spp., Peronospora spp., Pseudoperonospora spp. Bremia spp). Outstanding activity is observed against powdery mildews (e.g. Erysiphe necator) and leaf spots (e.g. Mycosphaerella spp.). Furthermore, the novel compounds of formula I are effective against phytopathogenic gram negative and gram positive bacteria (e.g. Xanthomonas spp, Pseudomonas spp, Erwinia amylovora, Ralstonia spp.) and viruses (e.g. tobacco mosaic virus).

The compounds of the present invention are suitable for controlling microbial (fungal) disease on a number of plants and their propagation material including, but not limited to the following target crops: cereals (wheat, barley, rye, oats, maize (including field corn, pop corn and sweet corn), rice, sorghum and related crops); beet (sugar beet and fodder beet); pomes, drupes and soft fruit (apples, pears, plums, peaches, almonds, cherries, strawberries, raspberries and blackberries); leguminous plants (beans, lentils, peas, soybeans); oil plants (rape, mustard, sunflowers, poppy, olives, coconut, castor oil plants, cocoa beans and groundnuts); cucumber plants (pumpkins, marrows, cucumbers, melons); fibre plants (cotton, flax, hemp, jute); vegetables (spinach, lettuce, asparagus, cabbages, carrots, eggplants, onions, pepper, tomatoes, potatoes, paprika, okra); plantation crops (bananas, fruit trees (e.g. oranges, lemons, grapefruit, mandarins), rubber trees, tree nurseries); lauraceae (avocado, cinnamomum, camphor) ornamentals (flowers, shrubs, broad-leaved trees and evergreens, such as conifers); as well as other plants such as vines, bushberries (such as blueberries), caneberries, cranberries, peppermint, rhubarb, spearmint, sugar cane, tobacco, nuts, coffee, eggplants, tea, pepper, bvines, hops and turf grasses including, but not limited to, cool-season turf grasses (for example, bluegrasses (Poa L.), such as Kentucky bluegrass (Poa pratensis L.), rough bluegrass (Poa trivialis L.), Canada bluegrass (Poa compressa L.) and annual bluegrass (Poa annua L.); bentgrasses (Agrostis L.), such as creeping bentgrass (Agrostis palustris Huds.), colonial bentgrass (Agrostis tenius Sibth.), velvet bentgrass (Agrostis canina L.) and redtop (Agrostis alba L.); fescues (Festuca L.), such as tall fescue (Festuca arundinacea Schreb.), meadow fescue (Festuca elation L.) and fine fescues such as creeping red fescue (Festuca rubra L.), chewings fescue (Festuca rubra var. commutata Gaud.), sheep fescue (Festuca ovina L.) and hard fescue (Festuca longifolia); and ryegrasses (Lolium L.), such as perennial ryegrass (Lolium perenne L.) and annual (Italian) ryegrass (Lolium multiflorum Lam.)) and warm-season turf grasses (for example, Bermudagrasses (Cynodon L. C. Rich), including hybrid and common Bermudagrass; Zoysiagrasses (Zoysia Willd.), St. Augustinegrass (Stenotaphrum secundatum (Walt.) Kuntze); and centipedegrass (Eremochloa ophiuroides (Munro.) Hack.)).

In addition ‘crops’ are to be understood to include those crops that have been made tolerant to pests and pesticides, including crops which are insect resistant or disease resistant as well as crops which are tolerant to herbicides or classes of herbicides, as a result of conventional methods of breeding or genetic engineering. Tolerance to e.g. herbicides means a reduced susceptibility to damage caused by a particular herbicide compared to conventional crop breeds. Crops can be modified or bred so as to be tolerant, for example, to HPPD inhibitors such as mesotrione or EPSPS inhibitors such as glyphosate.

The compounds of formula I may be in unmodified form or, preferably, may be incorporated into microbicidal (fungicidal) compositions. Typically the compounds of formula I are therefore formulated together with carriers and adjuvants conventionally employed in the art of formulation, using methods well known to the person skilled in the field of formulation.

The invention therefore also relates to a composition for the control of microbial (fungal) infection comprising a compound of formula I and an agriculturally acceptable carrier or diluent.

The agrochemical composition will usually contain from 0.1 to 99% by weight, preferably from 0.1 to 95% by weight, of the compound of formula I, 99.9 to 1% by weight, preferably 99.8 to 5% by weight, of a solid or liquid adjuvant, and from 0 to 25% by weight, preferably from 0.1 to 25% by weight, of a surfactant.

Rates and frequency of use of the formulations are those conventionally used in the art and will depend on the risk of infestation by the pathogen, the developmental stage of the plant and on the location, timing and application method. Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient rates of application are from 10 mg to 1 g of active substance per kg of seeds.

In practice, as indicated above, the agrochemical compositions comprising compound of formula I are applied as a formulation containing the various adjuvants and carriers known to or used in the industry. They may thus be formulated as granules, as wettable or soluble powders, as emulsifiable concentrates, as coatable pastes, as dusts, as flowables, as solutions, as suspensions or emulsions, or as controlled release forms such as microcapsules. These formulations are described in more detail below and may contain as little as about 0.5% to as much as about 95% or more by weight of the active ingredient. The optimum amount will depend on formulation, application equipment and nature of the plant pathogenic microbe to be controlled.

Suspension concentrates are aqueous formulations in which finely divided solid particles of the active compound are suspended. Such formulations include anti-settling agents and dispersing agents and may further include a wetting agent to enhance activity as well an anti-foam and a crystal growth inhibitor. In use, these concentrates are diluted in water and normally applied as a spray to the area to be treated. The amount of active ingredient may range from about 0.5% to about 95% of the concentrate.

Wettable powders are in the form of finely divided particles which disperse readily in water or other liquid carriers. The particles contain the active ingredient retained in a solid matrix. Typical solid matrices include fuller's earth, kaolin clays, silicas and other readily wet organic or inorganic solids. Wettable powders normally contain about 5% to about 95% of the active ingredient plus a small amount of wetting, dispersing or emulsifying agent.

Emulsifiable concentrates are homogeneous liquid compositions dispersible in water or other liquid and may consist entirely of the active compound with a liquid or solid emulsifying agent, or may also contain a liquid carrier, such as xylene, heavy aromatic naphthas, isophorone and other non-volatile organic solvents. In use, these concentrates are dispersed in water or other liquid and normally applied as a spray to the area to be treated. The amount of active ingredient may range from about 0.5% to about 95% of the concentrate.

Granular formulations include both extrudates and relatively coarse particles and are usually applied without dilution to the area in which control of plant pathogenic microbe is required. Typical inert carriers for granular formulations include sand, fuller's earth, attapulgite clay, bentonite clays, montmorillonite clay, vermiculite, perlite, calcium carbonate, brick, pumice, pyrophyllite, kaolin, dolomite, plaster, wood flour, ground corn cobs, ground peanut hulls, sugars, sodium chloride, sodium sulphate, sodium silicate, sodium borate, magnesia, mica, iron oxide, zinc oxide, titanium oxide, antimony oxide, cryolite, gypsum, diatomaceous earth, calcium sulphate and other organic or inorganic materials which absorb or which can be coated with the active compound. In addition, the inert granular carrier can be partially or wholly replaced by a granular fertilizer material. Granular formulations normally contain about 5% to about 25% active ingredients which may include surface-active agents such as heavy aromatic naphthas, kerosene and other petroleum fractions, or vegetable oils; and/or stickers such as dextrins, glue or synthetic resins.

Dusts are free-flowing admixtures of the active ingredient with finely divided solids such as talc, clays, flours and other organic and inorganic solids which act as dispersants and carriers.

Microcapsules are typically droplets or granules of the active ingredient enclosed in an inert porous shell which allows escape of the enclosed material to the surroundings at controlled rates. Encapsulated droplets are typically about 1 to 50 microns in diameter. The enclosed liquid typically constitutes about 50 to 95% of the weight of the capsule and may include solvent in addition to the active compound. Encapsulated granules are generally porous granules with porous membranes sealing the granule pore openings, retaining the active species in liquid form inside the granule pores. Granules typically range from 1 millimetre to 1 centimetre and preferably 1 to 2 millimetres in diameter. Granules are formed by extrusion, agglomeration or prilling, or are naturally occurring. Examples of such materials are vermiculite, sintered clay, kaolin, attapulgite clay, sawdust and granular carbon. Shell or membrane materials include natural and synthetic rubbers, cellulosic materials, styrene-butadiene copolymers, polyacrylonitriles, polyacrylates, polyesters, polyamides, polyureas, polyurethanes and starch xanthates.

Other useful formulations for agrochemical applications include simple solutions of the active ingredient in a solvent in which it is completely soluble at the desired concentration, such as acetone, alkylated naphthalenes, xylene and other organic solvents. Pressurised sprayers, wherein the active ingredient is dispersed in finely-divided form as a result of vaporisation of a low boiling dispersant solvent carrier, may also be used.

Suitable agricultural adjuvants and carriers that are useful in formulating the compositions of the invention in the formulation types described above are well known to those skilled in the art. Suitable examples of the different classes are found in the non-limiting list below.

Liquid carriers that can be employed include water, toluene, xylene, petroleum naphtha, crop oil, acetone, methyl ethyl ketone, cyclohexanone, acetic anhydride, acetonitrile, acetophenone, amyl acetate, 2-butanone, chlorobenzene, cyclohexane, cyclohexanol, alkyl acetates, diacetonalcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethyl formamide, dimethyl sulfoxide, 1,4-dioxane, dipropylene glycol, dipropylene glycol methyl ether, dipropylene glycol dibenzoate, diproxitol, alkyl pyrrolidinone, ethyl acetate, 2-ethyl hexanol, ethylene carbonate, 1,1,1-trichloroethane, 2-heptanone, alpha pinene, d-limonene, ethylene glycol, ethylene glycol butyl ether, ethylene glycol methyl ether, gamma-butyrolactone, glycerol, glycerol diacetate, glycerol monoacetate, glycerol triacetate, hexadecane, hexylene glycol, isoamyl acetate, isobornyl acetate, isooctane, isophorone, isopropyl benzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octyl amine acetate, oleic acid, oleylamine, o-xylene, phenol, polyethylene glycol (PEG400), propionic acid, propylene glycol, propylene glycol monomethyl ether, p-xylene, toluene, triethyl phosphate, triethylene glycol, xylene sulfonic acid, paraffin, mineral oil, trichloroethylene, perchloroethylene, ethyl acetate, amyl acetate, butyl acetate, methanol, ethanol, isopropanol, and higher molecular weight alcohols such as amyl alcohol, tetrahydrofurfuryl alcohol, hexanol, octanol, etc. ethylene glycol, propylene glycol, glycerine, N-methyl-2-pyrrolidinone, and the like. Water is generally the carrier of choice for the dilution of concentrates.

Suitable solid carriers include talc, titanium dioxide, pyrophyllite clay, silica, attapulgite clay, kieselguhr, chalk, diatomaxeous earth, lime, calcium carbonate, bentonite clay, fuller's earth, cotton seed hulls, wheat flour, soybean flour, pumice, wood flour, walnut shell flour, lignin and the like.

A broad range of surface-active agents are advantageously employed in both said liquid and solid compositions, especially those designed to be diluted with carrier before application. These agents, when used, normally comprise from 0.1% to 15% by weight of the formulation. They can be anionic, cationic, non-ionic or polymeric in character and can be employed as emulsifying agents, wetting agents, suspending agents or for other purposes. Typical surface active agents include salts of alkyl sulfates, such as diethanolammonium lauryl sulphate; alkylarylsulfonate salts, such as calcium dodecylbenzenesulfonate; alkylphenol-alkylene oxide addition products, such as nonylphenol-C.sub. 18 ethoxylate; alcohol-alkylene oxide addition products, such as tridecyl alcohol-C.sub. 16 ethoxylate; soaps, such as sodium stearate; alkylnaphthalenesulfonate salts, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; and salts of mono and dialkyl phosphate esters.

Other adjuvants commonly utilized in agricultural compositions include crystallisation inhibitors, viscosity modifiers, suspending agents, spray droplet modifiers, pigments, antioxidants, foaming agents, anti-foaming agents, light-blocking agents, compatibilizing agents, antifoam agents, sequestering agents, neutralising agents and buffers, corrosion inhibitors, dyes, odorants, spreading agents, penetration aids, micronutrients, emollients, lubricants, sticking agents, and the like.

In addition, further, other biocidally active ingredients or compositions may be combined with the compound of formula I and used in the methods of the invention and applied simultaneously or sequentially with the compound of formula I. When applied simultaneously, these further active ingredients may be formulated together with the compound of formula I or mixed in, for example, the spray tank. These further biocidally active ingredients may be fungicides, herbicides, insecticides, bactericides, acaricides, nematicides and/or plant growth regulators. Accordingly, the present invention provides a composition comprising (i) a compound of formula I and a further fungicide, (ii) a compound of formula I and a herbicide, (iii) a compound of formula I and an insecticide, (iv) a compound of formula I and a bactericide; (v) a compound of formula I and an acaricide, (vi) a compound of formula I and a nematicide and/or (vii) a compound of formula I and a plant growth regulator. In addition, the compounds of the invention may also be applied with one or more systemically acquired resistance inducers (“SAW” inducer). SAR inducers are known and described in, for example, U.S. Pat. No. 6,919,298 and include, for example, salicylates and the commercial SAR inducer acibenzolar-S-methyl.

The amount of the mixture and a further, other biocidally active ingredients or compositions combined with the compound of formula I to be applied, will depend on various factors, such as the compounds employed; the subject of the treatment, such as, for example plants, soil or seeds; the type of treatment, such as, for example spraying, dusting or seed dressing; the purpose of the treatment, such as, for example prophylactic or therapeutic; the type of fungi to be controlled or the application time.

It has been found that the use of a further, other biocidally active ingredients or compositions in combination with the compound of formula I surprisingly and substantially enhance the effectiveness of the latter against fungi, and vice versa. Additionally, the method of the invention is effective against a wider spectrum of such fungi that can be combated with the active ingredients of this method, when used solely.

The active ingredient mixture comprises compounds of formula I and a further, other biocidally active ingredients or compositions preferably in a mixing ratio of from 1000:1 to 1:1000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are understood to include, on the one hand, ratios by weight and also, on other hand, molar ratios.

A synergistic activity of the combination is apparent from the fact that the fungicidal activity of the composition of compounds of formula I and a further, other biocidally active ingredients or compositions is greater than the sum of the fungicidal activities of compounds of formula I and a further, other biocidally active ingredients or compositions.

The method of the invention comprises applying to the useful plants, the locus thereof or propagation material thereof in admixture or separately, a synergistically effective aggregate amount of a compound of formula I and a further, other biocidally active ingredients or compositions.

Some of said combinations according to the invention have a systemic action and can be used as foliar, soil and seed treatment fungicides.

With the combinations according to the invention it is possible to inhibit or destroy the phytopathogenic microorganisms which occur in plants or in parts of plants (fruit, blossoms, leaves, stems, tubers, roots) in different useful plants, while at the same time the parts of plants which grow later are also protected from attack by phytopathogenic microorganisms.

The combinations of the present invention are of particular interest for controlling a large number of fungi in various useful plants or their seeds, especially in field crops such as potatoes, tobacco and sugarbeets, and wheat, rye, barley, oats, rice, maize, lawns, cotton, soybeans, oil seed rape, pulse crops, sunflower, coffee, sugarcane, fruit and ornamentals in horticulture and viticulture, in vegetables such as cucumbers, beans and cucurbits.

The combinations according to the invention are applied by treating the fungi, the useful plants, the locus thereof, the propagation material thereof, the natural substances of plant and/or animal origin, which have been taken from the natural life cycle, and/or their processed forms, or the industrial materials threatened by fungus attack with a combination compounds of formula I and a further, other biocidally active ingredients or compositions in a synergistically effective amount.

The combinations according to the invention may be applied before or after infection of the useful plants, the propagation material thereof, the natural substances of plant and/or animal origin, which have been taken from the natural life cycle, and/or their processed forms, or the industrial materials by the fungi.

In particular, the composition of the invention comprises at least one additional fungicidally active compound in addition to the compound of formula (I). Preferably, the composition of the invention comprises one additional fungicidally active compound or two or three or more additional fungicidally active compounds in addition to the compound of formula (I)

In particular, composition encompassed by the present invention include, but are not limited to, compositions comprising a compound of formula I and acibenzolar-S-methyl (CGA245704), a compound of formula I and ancymidol, a compound of formula I and alanycarb, a compound of formula I and aldimorph, a compound of formula I and amisulbrom, a compound of formula I and anilazine, a compound of formula I and azaconazole, a compound of formula I and azoxystrobin, a compound of formula I and BAY 14120, a compound of formula I and benalaxyl, a compound of formula I and benthiavalicarb, a compound of formula I and benomyl, a compound of formula I and biloxazol, a compound of formula I and bitertanol, a compound of formula I and bixafen, a compound of formula I and blasticidin S, a compound of formula I and boscalid, a compound of formula I and bromuconazole, a compound of formula I and bupirimate, a compound of formula I and captafol, a compound of formula I and captan, a compound of formula I and carbendazim, a compound of formula I and carbendazim, a compound of formula I and chlorhydrate, a compound of formula I and carboxin, a compound of formula I and carpropamid, a compound of formula I and carvone, a compound of formula I and CGA41396, a compound of formula I and CGA41397, a compound of formula I and chinomethionate, a compound of formula I and chloroneb, a compound of formula I and chlorothalonil, a compound of formula I and chlorozolinate, a compound of formula I and clozylacon, a compound of formula I and copper containing compounds (e.g. a compound of formula I and copper oxychloride, a compound of formula I and cuprous oxide, a compound of formula I and mancopper, a compound of formula I and oxine-copper a compound of formula I and copper hydroxide, a compound of formula I and copper oxyquinolate, a compound of formula I and copper sulphate, a compound of formula I and copper tallate and a compound of formula I and Bordeaux mixture), a compound of formula I and cyflufenamid, a compound of formula I and cymoxanil, a compound of formula I and cyproconazole, a compound of formula I and cyprodinil, a compound of formula I and debacarb, a compound of formula I and di-2-pyridyl disulphide 1,1′-dioxide, a compound of formula I and dichlofluanid, a compound of formula I and diclomezine, a compound of formula I and dichlozoline, a compound of formula I and dichlone, a compound of formula I and dicloran, a compound of formula I and diclocymet, a compound of formula I and diethofencarb, a compound of formula I and difenoconazole, a compound of formula I and difenzoquat, a compound of formula I and diflumetorim, a compound of formula I and O,O-di-iso-propyl-S-benzyl thiophosphate, a compound of formula I and dimefluazole, a compound of formula I and dimetconazole, a compound of formula I and dimethomorph, a compound of formula I and dimethirimol, a compound of formula I and dimoxystrobin, a compound of formula I and diniconazole, a compound of formula I and dinocap, a compound of formula I and dithianon, a compound of formula I and dodecyl dimethyl ammonium chloride, a compound of formula I and dodemorph, a compound of formula I and dodine, a compound of formula I and doguadine, a compound of formula I and edifenphos, a compound of formula I and enestrobin, a compound of formula I and epoxiconazole, a compound of formula I and ethaboxam, a compound of formula I and ethirimol, a compound of formula I and etridiazole, a compound of formula I and famoxadone, a compound of formula I and fenamidone (RPA407213), a compound of formula I and fenarimol, a compound of formula I and fenbuconazole, a compound of formula I and fenfuram, a compound of formula I and fenhexamid (KBR2738), a compound of formula I and fenoxanil, a compound of formula I and fenpiclonil, a compound of formula I and fenpropidin, a compound of formula I and fenpropimorph, a compound of formula I and fentin acetate, a compound of formula I and fentin hydroxide, a compound of formula I and ferbam, a compound of formula I and ferimzone, a compound of formula I and fluazinam, a compound of formula I and fluopicolide, a compound of formula I and fludioxonil, a compound of formula I and fluoxastrobin, a compound of formula I and flumetover, a compound of formula I and SYP-LI90 (flumorph), a compound of formula I and fluopyram, a compound of formula I and fluoroimide, a compound of formula I and fluquinconazole, a compound of formula I and flusilazole, a compound of formula I and flusulfamide, a compound of formula I and flutolanil, a compound of formula I and flutriafol, a compound of formula I and folpet, a compound of formula I and fosetyl-aluminium, a compound of formula I and fuberidazole, a compound of formula I and furalaxyl, a compound of formula I and furametpyr, a compound of formula I and guazatine, a compound of formula I and hexaconazole, a compound of formula I and hydroxyisoxazole, a compound of formula I and hymexazole, a compound of formula I and IKF-916 (cyazofamid), a compound of formula I and imazalil, a compound of formula I and imibenconazole, a compound of formula I and iminoctadine, a compound of formula I and iminoctadine triacetate, a compound of formula I and ipconazole, a compound of formula I and iprobenfos, a compound of formula I and iprodione, a compound of formula I and iprovalicarb (SZX0722), a compound of formula I and isopropanyl butyl carbamate, a compound of formula I and isoprothiolane, a compound of formula I and kasugamycin, a compound of formula I and kresoxim-methyl, a compound of formula I and LY186054, a compound of formula I and LY211795, a compound of formula I and LY248908, a compound of formula I and maneb, a compound of formula I and mancopper, a compound of formula I and mancozeb, a compound of formula I and mandipropamid, a compound of formula I and mefenoxam, a compound of formula I and mepanipyrim, a compound of formula I and mepronil, a compound of formula I and metalaxyl, a compound of formula I and metconazole, a compound of formula I and methasulfocarb, a compound of formula I and metiram, a compound of formula I and metiram-zinc, a compound of formula I and metominostrobin, a compound of formula I and metrafenone, a compound of formula I and myclobutanil, a compound of formula I and myclozoline, a compound of formula I and neoasozin, a compound of formula I and nickel dimethyldithiocarbamate, a compound of formula I and nicobifen, a compound of formula I and nitrothal-isopropyl, a compound of formula I and nuarimol, a compound of formula I and ofurace, a compound of formula I and organomercury compounds, a compound of formula I and orysastrobin, a compound of formula I and oxadixyl, a compound of formula I and oxasulfuron, a compound of formula I and oxine-copper, a compound of formula I and oxolinic acid, a compound of formula I and oxpoconazole, a compound of formula I and oxycarboxin, a compound of formula I and pefurazoate, a compound of formula I and penconazole, a compound of formula I and pencycuron, a compound of formula I and penthiopyrad, a compound of formula I and phenazin oxide, a compound of formula I and phosdiphen, a compound of formula I and phosphorus acids, a compound of formula I and phthalide, a compound of formula I and picoxystrobin (ZA1963), a compound of formula I and polyoxin D, a compound of formula I and polyram, a compound of formula I and probenazole, a compound of formula I and prochloraz, a compound of formula I and procymidone, a compound of formula I and propamocarb, a compound of formula I and propiconazole, a compound of formula I and propineb, a compound of formula I and propionic acid, a compound of formula I and proquinazid, a compound of formula I and prothioconazole, a compound of formula I and pyraclostrobin, a compound of formula I and pyrazophos, a compound of formula I and pyribencarb, a compound of formula I and pyrifenox, a compound of formula I and pyrimethanil, a compound of formula I and pyroquilon, a compound of formula I and pyroxyfur, a compound of formula I and pyrrolnitrin, a compound of formula I and quaternary ammonium compounds, a compound of formula I and quinomethionate, a compound of formula I and quinoxyfen, a compound of formula I and quintozene, a compound of formula I and silthiofam, a compound of formula I and simeconazole, a compound of formula I and sipconazole (F-155), a compound of formula I and sodium pentachlorophenate, a compound of formula I and spiroxamine, a compound of formula I and streptomycin, a compound of formula I and sulphur, a compound of formula I and schwefel, a compound of formula I and tebuconazole, a compound of formula I and tecloftalam, a compound of formula I and tecnazene, a compound of formula I and tetraconazole, a compound of formula I and thiabendazole, a compound of formula I and thifluzamid, a compound of formula I and 2-(thiocyanomethylthio)benzothiazole, a compound of formula I and thiophanatemethyl, a compound of formula I and thiram, a compound of formula I and tiadinil, a compound of formula I and timibenconazole, a compound of formula I and tolclofos-methyl, a compound of formula I and tolylfluanid, a compound of formula I and triadimefon, a compound of formula I and triadimenol, a compound of formula I and triazbutil, a compound of formula I and triazoxide, a compound of formula I and tricyclazole, a compound of formula I and tridemorph, a compound of formula I and trifloxystrobin (CGA279202), a compound of formula I and triforine, a compound of formula I and triflumizole, a compound of formula I and triticonazole, a compound of formula I and validamycin A, a compound of formula I and vapam, a compound of formula I and valiphenal a compound of formula I and vinclozolin, a compound of formula I and zineb, a compound of formula I and ziram, a compound of formula I and zoxamide, a compound of formula I and 3-[5-(4-chlorophenyl)-2,3-dimethylisoxazolidin-3-yl]pyridine, a compound of formula I and 5-chloro-7-(4-methylpiperidine-1-yl)-6-(2,4,6-trifluorophenyl)[1,2,4]triazolo[1,5-a]pyrimidine, a compound of formula I and N-(4-chloro-2-nitrophenyl)-N-ethyl-4-methyl-benzsulfonamide, a compound of formula I and 3-difluoromethyl-1-methyl-1H-pyrazole-4-carboxylic acid (9-isopropyl-1,2,3,4-tetrahydro-1,4-methano-naphthalen-5-yl)-amide (isopyrazam), a compound of formula I and 3-difluoromethyl-1-methyl-1H-pyrazole-4-carboxylic acid (2-bicyclopropyl-2-yl-phenyl)-amide (sedaxane), a compound of formula I and N-(3′,4′-dichloro-5-fluoro-1,1′-biphenyl-2-yl)-3-(difluoromethyl)-1-methyl-1H-pyrazole-4-carboxamide and a compound of formula I and glyphosate

More particularly, the composition according to the present invention comprises a compound of formula I and acibenzolar-S-methyl, a compound of formula I and azoxystrobin, a compound of formula I and chlorothalonil, a compound of formula I and cyproconazole, a compound of formula I and cyprodinil, a compound of formula I and difenoconazole, a compound of formula I and fenpropidin, a compound of formula I and fluazinam, a compound of formula I and fludioxonil, a compound of formula I and hexaconazole, a compound of formula I and isopyrazam, a compound of formula I and mandipropamid, a compound of formula I and mefenoxam, a compound of formula I and penconazole, a compound of formula I and propiconazole, a compound of formula I and pyroquilon, a compound of formula I and sedaxane or a compound of formula I and thiabendazole.

The formulations of the invention and for use in the methods of the invention can be applied to the areas where control is desired by conventional methods such as spraying, atomising, dusting, scattering, coating or pouring. Dust and liquid compositions, for example, can be applied by the use of power-dusters, broom and hand sprayers and spray dusters. The formulations can also be applied from airplanes as a dust or a spray or by rope wick applications. One method of applying the formulation of the invention is foliar application. In addition, both solid and liquid formulations may also be applied to the soil in the locus of the plant to be treated allowing the active ingredient to penetrate the plant through the roots. The formulations of the invention may also be used for dressing applications on plant propagation material to provide protection against microbial (fungal) infections on the plant propagation material as well as against phytopathogenic microbes (fungi) occurring in the soil. Suitably, the active ingredient may be applied to plant propagation material to be protected by impregnating the plant propagation material, in particular, seeds, either with a liquid formulation or coating it with a solid formulation. In special cases, other types of application are also possible, for example, the specific treatment of plant cuttings or twigs serving propagation. It is noted that, whereas it is preferred to formulate commercial products as concentrates, the end user will normally use dilute formulations.

Furthermore, the compounds of formula I find general use as fungicides and may therefore also be used in methods to control pathogenic fungi in related areas, for example in the protection of technical materials, in food storage or in hygiene management. As such, the present invention further provides the use of a compound of formula I for preventing and/or controlling fungal infection on technical materials, in food storage or in hygiene management. In addition, the present invention also provides a method for controlling and/or preventing infestation of technical materials by fungi comprising applying the compound of formula I to the technical material or the locus thereof in a fungicidally effective amount.

“Technical materials” include but are not limited to organic and inorganic materials such as wood, paper, leather, natural and synthetic fibers, composites thereof such as particle board, plywood, wall-board and the like, woven and non-woven fabrics, construction surfaces and materials (e.g. building material), cooling and heating system surfaces and materials, ventilation and air conditioning system surfaces and materials, and the like. The compounds and combinations according the present invention can be applied to such materials or surfaces in an amount effective to inhibit or prevent disadvantageous effects such as decay, discoloration or mold in like manner as described above. Structures and dwellings constructed using or incorporating technical materials in which such compounds or combinations have been applied are likewise protected against attack by fungi

The technical material can be treated with a compound of formula I in a number of ways, including, but not limited to, by including the compound in the technical material itself, absorbing, impregnating, treating (in closed pressure or vacuum systems) said material with said compound, dipping or soaking the building material, or coating the material for example by curtain coating, roller, brush, spray, atomisation, dusting, scattering or pouring application. The compound of the invention can be formulated for use in treatment of technical materials by using techniques well known to the person skilled in the art. Such formulations may utilise, for example, the formulation materials listed above in relation to agrochemical formulations.

Furthermore, the compounds of the present invention may find use as plant growth regulators or in plant health applications.

Plant growth regulators (PGRs) are generally any substances or mixtures of substances intended to accelerate or retard the rate of growth or maturation, or otherwise alter the development of plants or their produce.

Plant growth regulators (PGRs) affect growth and differentiation of plants.

More specifically, various plant growth regulators (PGRs) can, for example, reduce plant height, stimulate seed germination, induce flowering, darken leaf coloring, change the rate of plant growth and modify the timing and efficiency of fruiting.

Plant health applications include, for example, improvement of advantageous properties/crop characteristics including: emergence, crop yields, protein content, increased vigour, faster maturation, increased speed of seed emergence, improved nitrogen utilization efficiency, improved water use efficiency, improved oil content and/or quality, improved digestibility, faster ripening, improved flavor, improved starch content, more developed root system (improved root growth), improved stress tolerance (e.g. against drought, heat, salt, light, UV, water, cold), reduced ethylene (reduced production and/or inhibition of reception), tillering increase, increase in plant height, bigger leaf blade, less dead basal leaves, stronger tillers, greener leaf color, pigment content, photosynthetic activity, less input needed (such as fertilizers or water), less seeds needed, more productive tillers, earlier flowering, early grain maturity, less plant verse (lodging), increased shoot growth, enhanced plant vigor, increased plant stand and early and better germination.

Advantageous properties, obtained especially from treaded seeds, are e.g. improved germination and field establishment, better vigor, more homogeneous field establishment.

Advantageous properties, obtained especially from foliar and/or in-furrow application are e.g. improved plant growth and plant development, better growth, more tillers, greener leafs, larger leaves, more biomass, better roots, improved stress tolerance of the plants, more grain yield, more biomass harvested, improved quality of the harvest (content of fatty acids, metabolites, oil etc), more marketable products (e.g. improved size), improved process (e.g. longer shelf-life, better extraction of compounds), improved quality of seeds (for being seeded in the following seasons for seed production); or any other advantages familiar to a person skilled in the art.

The term plant health thus comprises various sorts of improvements of plants that are not connected to the control of harmful microbes.

The present invention will now be described by way of the following non-limiting examples. Those skilled in the art will promptly recognize appropriate variations from the procedures both as to reactants and as to reaction conditions and techniques

### EXAMPLES

### Example 1

### The preparation of 2-[6-(3-fluoro-4-methoxy-phenyl)-5-methylpyridin-2-yl]-quinazoline (Compound Table 3/Entry 92)

### a) Preparation of 2-(3-fluoro-4-methoxyphenyl)-3-methylpyridine

3-Fluoro-4-methoxyphenylboronic acid (14.8 g, 87.2 mmol) and 77.5 ml of a sodium carbonate solution (3 M in water) are added to solution of 2-bromo-3-methylpyridine (10 g, 58 mmol) in 600 ml of 1,2-dimethoxyethane. After degassing this mixture with argon for 15 min, [1,1-bis(diphenylphosphino)ferrocene]dichloropalladium(II), complex with dichloromethane (950 mg, 1.1 mmol) is added and the reaction mixture is stirred for 2 h at 95° C. Subsequently the reaction mixture is cooled, diluted with water and extracted with ethyl acetate. The combined organic layer is washed with sodium hydroxide solution (1 M in water) and brine, dried over sodium sulfate and evaporated under reduced pressure. The remainder is purified by chromatography on silica gel, using a mixture of cyclohexane/ethyl acetate 2:1 as eluent to obtain 2-(3-fluoro-4-methoxyphenyl)-3-methylpyridine. ¹H-NMR (CDCl₃): δ=2.40 (s, 3H), 3.97 (s, 3H), 7.06 (t, 1H), 7.19 (dd, 1H), 7.28-7.35 (m, 2H), 7.59 (d, 1H), 8.53 (d, 1H). MS: m/z=218 (M+1).

### b) Preparation of 2-(3-fluoro-4-methoxyphenyl)-3-methylpyridine 1-oxide

3-Chloroperbenzoic acid (21.5 g, 87.5 mmol) is added to a solution of 2-(3-fluoro-4-methoxyphenyl)-3-methylpyridine (9.5 g, 44 mmol) in 95 ml of dichloromethane. The reaction mixture is stirred for 16 h at room temperature and extracted with sodium hydroxide solution (2 M in water). The organic layer is then washed with aqueous sodium thiosulfate solution, sodium hydroxide solution (1 M in water) and brine, dried over sodium sulfate and evaporated under reduced pressure to obtain 2-(3-fluoro-4-methoxyphenyl)-3-methylpyridine 1-oxide, which can be used in the next step without further purification. ¹H-NMR (CDCl₃): δ=2.15 (s, 3H), 3.95 (s, 3H), 7.08-7.21 (m, 5H), 8.24 (d, 1H). MS: m/z=234 (M+1).

### c) Preparation of 6-(3-fluoro-4-methoxyphenyl)-5-methylpyridine-2-carbonitrile

Trimethylsilylcyanide (4.6 g, 47 mmol) is added to a solution of 2-(3-fluoro-4-methoxyphenyl)-3-methylpyridine 1-oxide (8.8 g, 38 mmol) in 135 ml of 1,2-dichloroethane. The resulting solution is stirred for 1 h at room temperature. Subsequently, N,N-dimethylcarbamoyl chloride (5.0 g, 47 mmol) is added slowly within 30 min. The reaction mixture is stirred for 16 h at 65° C., then quenched by slow addition of water. After phase separation, the organic layer is washed with sodium hydroxide solution (2 M in water) and water, dried over sodium sulfate and evaporated under reduced pressure. The remainder is purified by chromatography on silica gel, using a mixture of cyclohexane/ethyl acetate 3:1 as eluent to obtain 6-(3-fluoro-4-methoxyphenyl)-5-methylpyridine-2-carbonitrile. ¹H-NMR (CDCl₃): δ=2.48 (s, 3H), 3.98 (s, 3H), 7.07 (t, 1H), 7.30-7.36 (m, 2H), 7.59 (dd, 1H), 7.72 (dd, 1H). MS: m/z=243 (M+1).

### d) Preparation of 2-[6-(3-fluoro-4-methoxyphenyl)-5-methylpyridin-2-yl]-3H-quinazolin-4-one

7.6 ml of a sodium methoxide solution (5.4 M in methanol) are added to a suspension of 6-(3-fluoro-4-methoxyphenyl)-5-methylpyridine-2-carbonitrile (5.0 g, 20 mmol) in 50 ml of methanol. The resulting mixture is stirred for 2 h at 65° C. Subsequently, anthranilic acid (8.7 g, 64 mmol) is added and the reaction mixture is stirred for 16 h at 95° C., then cooled, diluted with ethyl acetate and extracted with sodium hydroxide solution (2 M in water). The combined organic layer is then washed with brine, dried over sodium sulfate and evaporated under reduced pressure. The residue is taken up in 15 ml of dichloromethane, stirred for 10 min and filtered to obtain 2-[6-(3-fluoro-4-methoxyphenyl)-5-methylpyridin-2-yl]-3H-quinazolin-4-one. ¹H-NMR (d₆-DMSO): δ=2.51 (s, 3H), 3.94 (s, 3H), 7.29 (t, 1H), 7.55-7.64 (m, 2H), 7.82 (d, 1H), 7.88-8.01 (m, 3H), 8.20 (d, 1H), 8.32 (d, 1H). MS: m/z=362 (M+1).

### e) Preparation of 4-chloro-2-[6-(3-fluoro-4-methoxyphenyl)-5-methylpyridin-2-yl]-quinazoline

2-[6-(3-fluoro-4-methoxyphenyl)-5-methylpyridin-2-yl]-3H-quinazolin-4-one (2.5 g, 6.9 mmol) are stirred in 20 ml of phosphorous oxychloride for 1 h at 60° C. The reaction mixture is cooled and evaporated under reduced pressure. The residue is taken up in dichloromethane and extracted with sodium hydroxide solution (2 M in water). The organic layer is washed with brine, dried over sodium sulfate and evaporated under reduced pressure to obtain 4-chloro-2-[6-(3-fluoro-4-methoxyphenyl)-5-methylpyridin-2-yl]-quinazoline, which can be used in the next step without further purification. ¹H-NMR (CDCl₃): δ=2.44 (s, 3H), 3.92 (s, 3H), 7.31 (t, 1H), 7.47 (d, 1H), 7.55 (d, 1H), 7.82-8.02 (m, 3H), 8.13-8.22 (m, 2H), 8.43 (d, 1H). MS: m/z=380 (M+1).

### f) Preparation of 2-[6-(3-fluoro-4-methoxyphenyl)-5-methylpyridin-2-yl]-3,4-dihydroquinazoline

Palladium (10% on charcoal, 36 mg, 0.34 mmol) is added to a suspension of 4-chloro-2-[6-(3-fluoro-4-methoxyphenyl)-5-methylpyridin-2-yl]-quinazoline (2.6 g, 6.8 mmol) and triethylamine (4.1 g, 41 mmol) in 300 ml of methanol under argon. The argon is exchanged for hydrogen and the reaction mixture is stirred for 16 h at room temperature under hydrogen. Subsequently the reaction mixture is filtered through celite and evaporated under reduced pressure. The residue is taken up in dichloromethane and extracted with a saturated aqueous sodium hydrogen carbonate solution. The organic layer is washed with brine, dried over sodium sulfate and evaporated under reduced pressure to obtain 2-[6-(3-fluoro-4-methoxyphenyl)-5-methylpyridin-2-yl]-3,4-dihydroquinazoline, which can be used in the next step without further purification. ¹H-NMR (CDCl₃): δ=2.42 (s, 3H), 3.99 (s, 3H), 4.88 (bs, 1H), 5.32 (d, 2H), 7.02 (t, 1H), 7.05-7.13 (m, 4H), 7.21 (t, 1H), 7.32 (dd, 1H), 7.38 (dd, 1H), 7.76 (d, 1H). MS: m/z=348 (M+1).

### g) Preparation of 2-[6-(3-fluoro-4-methoxyphenyl)-5-methylpyridin-2-yl]-quinazoline (Table 3/Entry 92))

2,3-Dichloro-5,6-dicyano-p-benzoquinone (2.1 g, 9.2 mmol) is added to a suspension of 2-[6-(3-fluoro-4-methoxyphenyl)-5-methylpyridin-2-yl]-3,4-dihydroquinazoline (2.9 g, 8.4 mmol) in 150 ml of toluene. The reaction mixture is stirred for 30 min at room temperature, diluted with ethyl acetate and extracted with a saturated aqueous sodium hydrogen carbonate solution. The organic layer is washed with aqueous sodium thiosulfate solution and brine, dried over sodium sulfate and evaporated under reduced pressure. The remainder is purified by chromatography on silica gel, using a mixture of cyclohexane/ethyl acetate/dichloromethane 2:1:1 as eluent to obtain 2-[6-(3-fluoro-4-methoxyphenyl)-5-methylpyridin-2-yl]-quinazoline (Compound No I.a.581). ¹H-NMR (CDCl₃): δ=2.48 (s, 3H), 3.97 (s, 3H), 7.08 (t, 1H), 7.43 (dd, 1H), 7.49 (dd, 1H), 7.69 (t, 1H), 7.81 (d, 1H), 7.93-8.00 (m, 2H), 8.21 (d, 1H), 8.54 (d, 1H), 9.60 (s, 1H). MS: m/z=346 (M+1).

### Example 2

### This Example illustrates the preparation of 2-(6-benzylpyridin-2-yl)-quinazoline (Compound Table 6/Entry 17))

### a) Preparation of 2-(6-bromopyridin-2-yl)-1,2,3,4-tetrahydroquinazoline

A solution of pyridine (5.1 g, 64 mmol) in 50 ml of dichloromethane is added to a solution of thionyl chloride (7.6 g, 64 mmol) in 50 ml of dichloromethane at 0° C. The mixture is stirred for 15 min at 0° C., then 6-bromopyridine-2-carboxaldehyde (10 g, 54 mmol) is added slowly at 0° C. The resulting mixture is stirred for 1 h at room temperature, then a solution of 2-aminobenzylamine (7.2 g, 59 mmol) in 50 ml of dichloromethane is added dropwise. The reaction mixture is stirred for 1 h at room temperature, then diluted with 50 ml of a sodium acetate solution (8.8 g in water), basified with sodium hydroxide solution (2 M in water) and extracted with dichloromethane. The organic layer is washed with brine, dried over sodium sulfate and evaporated under reduced pressure. The remainder is purified by chromatography on silica gel, using a mixture of cyclohexane/ethyl acetate 2:1 as eluent to obtain 2-(6-bromopyridin-2-yl)-1,2,3,4-tetrahydroquinazoline. ¹H-NMR (CDCl₃): δ=4.02 (d, 1H), 4.27 (d, 1H), 5.01 (bs, 1H), 5.23 (s, 1H), 6.68-6.76 (m, 2H), 6.93 (d, 1H), 7.07 (t, 1H), 7.44 (d, 1H), 7.58-7.63 (m, 2H). MS: m/z=291 (M+1).

### b) Preparation of 2-(6-bromopyridin-2-yl)-quinazoline

2,3-Dichloro-5,6-dicyano-p-benzoquinone (121 g, 0.53 mol) is added to a suspension of 2-(6-bromopyridin-2-yl)-1,2,3,4-tetrahydroquinazoline (77 g, 0.26 mol) in 1450 ml of toluene. The reaction mixture is stirred for 30 min at room temperature, basified with sodium hydroxide solution (5 M in water) and extracted with ethyl acetate. The organic layer is washed with brine, dried over sodium sulfate and evaporated under reduced pressure. The remainder is purified by chromatography on silica gel, using a mixture of cyclohexane/ethyl acetate/dichloromethane 2:1:1 as eluent to obtain 2-(6-bromopyridin-2-yl)-quinazoline. ¹H-NMR (CDCl₃): δ=7.63 (d, 1H), 7.69-7.78 (m, 2H), 7.93-8.01 (m, 2H), 8.20 (d, 1H), 8.64 (d, 2H), 9.59 (s, 1H). MS: m/z=287 (M+1).

### c) Preparation of 2-(6-benzylpyridin-2-yl)-quinazoline

A solution of 2-(6-bromopyridin-2-yl)-quinazoline (9.0 g, 32 mmol) in 450 ml of tetrahydrofurane is degassed with argon for 10 min. Tetrakis(triphenylphosphin)palladium (0.36 g, 0.32 mmol) is added and the mixture is stirred for 30 min at 65° C. 70 ml of a benzylzinc bromide solution (0.5 M in tetrahydrofurane) are added and the reaction mixture is heated to reflux for 16 h. Subsequently the mixture is cooled and 250 ml of a EDTA solution (12% in water) are added and the mixture is stirred for further 72 h at room temperature, then diluted with sodium hydroxide solution (1 M in water) and extracted with ethyl acetate. The organic layer is washed with brine, dried over sodium sulfate and evaporated under reduced pressure. The remainder is purified by chromatography on silica gel, using a mixture of cyclohexane/ethyl acetate 2:1 as eluent to obtain 2-(6-benzylpyridin-2-yl)-quinazoline (Compound No I.a.017). ¹H-NMR (CDCl₃): δ=4.48 (s, 2H), 7.12 (d, 1H), 7.23-7.35 (m, 5H), 7.70 (t, 1H), 7.77 (t, 1H), 7.93-8.02 (m, 2H), 8.22 (d, 1H), 8.51 (d, 1H), 9.62 (s, 1H). MS: m/z=298 (M+1).

### Example 3

### This Example illustrates the preparation of 2-(6-o-tolyloxypyridin-2-yl)-quinazoline (Compound Table 4/Entry 22)

A mixture of 2-(6-bromopyridin-2-yl)-quinazoline (200 mg, 0.7 mmol), o-cresol (94 mg, 0.7 mmol), copper(I) bromide (20 mg, 0.14 mmol) and cesium carbonate (570 mg, 1.75 mmol) is degassed with argon. Then 2,2,6,6-tetramethyl-3,5-heptandion (103 mg, 0.56 mmol) and 2 ml of N,N-dimethylformamide are added and this mixture is heated in a sealed tube for 22 h at 135° C. Subsequently the mixture is cooled and 20 ml of a EDTA solution (12% in water) are added and the mixture is stirred for further 72 h at room temperature, then diluted with sodium hydroxide solution (1 M in water) and extracted with ethyl acetate. The organic layer is washed with brine, dried over sodium sulfate and evaporated under reduced pressure. The remainder is purified by chromatography on silica gel, using a mixture of cyclohexane/ethyl acetate 2:1 as eluent to obtain 2-(6-o-tolyloxypyridin-2-yl)-quinazoline (Compound No I.a.093). ¹H-NMR (CDCl₃): δ=2.28 (s, 3H), 6.63 (d, 1H), 7.12-7.31 (m, 4H), 7.64 (t, 1H), 7.80 (t, 1H), 7.89-7.95 (m, 2H), 8.22 (d, 1H), 8.39 (d, 1H), 9.57 (s, 1H). MS: m/z=314 (M+1).

### Example 4

### This Example illustrates the preparation of 2-[6-(4-chlorophenylsulfanyl)-pyridin-2-yl]-quinazoline (Compound Table 9/Entry 3)

A mixture of 2-(6-bromopyridin-2-yl)-quinazoline (200 mg, 0.7 mmol), 4-chlorothiophenol (139 mg, 0.77 mmol), N,N-dimethylformamide (128 mg, 1.75 mmol) and potassium carbonate (121 mg, 0.87 mmol) is heated under argon in a sealed tube for 3 h at 110° C. Subsequently the mixture is cooled, diluted with sodium hydroxide solution (1 M in water) and extracted with ethyl acetate. The organic layer is washed with brine, dried over sodium sulfate and evaporated under reduced pressure. The remainder is purified by chromatography on silica gel, using a mixture of cyclohexane/ethyl acetate 2:1 as eluent to obtain 2-[6-(4-chlorophenylsulfanyl)-pyridin-2-yl]-quinazoline (Compound No I.a.319). ¹H-NMR (CDCl₃): δ=6.92 (d, 1H), 7.43 (d, 2H), 7.58-7.70 (m, 4H), 7.92-8.01 (m, 2H), 8.21 (d, 1H), 8.39 (d, 1H), 9.60 (s, 1H). MS: m/z=350 (M+1).

### Example 5

### This Example illustrates the preparation of 4-Methyl-2-(5-methyl-6-phenyl-pyridin-2-yl)-quinazoline (Compound Table 11/Entry 8)

### a) Synthesis of 3-methyl-2-phenyl-pyridine

To a stirred solution of 2-bromo-3-methylpyridine (30 g, 174 mmol) in dimethoxyethane (1.3 L) was added in one portion phenylboronic acid (42.5 g, 349 mmol) at room temperature, followed by an aqueous solution of sodium carbonate (3 M in water, 233 mL, 698 mmol). The mixture was degassed with argon for about 30 minutes, after which [1,1′-bis(diphenylphosphino)ferrocene]dichloropalladium(II), complex with dichloromethane (4.3 g, 5.0 mmol) was added, under argon. The reaction was stirred at 95° C. for 2 hours. The crude mixture was diluted with ethyl acetate and water and the organic layer was decanted. It was washed once with an aqueous solution of sodium hydroxide (0.5 M) and once with brine. The organic layer was collected, dried with sodium sulphate and concentrated in vacuo. The crude mixture was purified by flash chromatography on silica gel (eluent: ethyl acetate/cyclohexane 1:3). The title compound was obtained as a pale orange oil. ¹H-NMR (CDCl₃): δ=2.37 (s, 3H), 7.19 (dd, 1H), 7.37-7.41 (m, 2H), 7.42-7.49 (dd, 1H), 7.52-7.56 (m, 2H), 7.60 (d, 1H), 8.55 (d, 1H).

### b) Synthesis of 3-methyl-2-phenyl-pyridine 1-oxide

To a stirred solution of 3-methyl-2-phenyl-pyridine (26.9 g, 159 mmol) in dichloromethane (220 mL) under nitrogen atmosphere was added m-chloroperbenzoic acid (70% pure, 78.4 g, 318 mmol) in small portions, at 0° C. The mixture was stirred overnight at room temperature. It was then cooled to 0° C. and an aqueous solution of sodium hydroxide (2 M) was added slowly (CAUTION: exothermic) until a basic pH was reached. To this mixture was then added a saturated aqueous solution of sodium thiosulphate slowly at 0° C. (CAUTION: highly exothermic). The biphasic solution was stirred for an additional 30 minutes after which the organic layer was decanted, washed with an aqueous solution of sodium hydroxide (1 M), decanted, dried over sodium sulphate and concentrated in vacuo. The crude compound was obtained as a white solid. ¹H-NMR (CDCl₃): δ=2.13 (s, 3H), 7.15-7.22 (m, 2H), 7.47 (d, 2H), 7.43-7.49 (m, 1H), 7.51-7.57 (m, 2H), 8.27 (d, 1H).

### c) Synthesis of 5-methyl-6-phenyl-1H-pyridin-2-one

A solution of 3-methyl-2-phenyl-pyridine 1-oxide (12 g, 65 mmol) in acetic anhydride (120 mL) was equally partitioned in four microwave vials and sealed. The vials were irradiated for 45 minutes in a microwave oven at 175° C. The crude mixture was concentrated in vacuo. The crude was taken up in ethyl acetate (100 mL) and an aqueous solution of lithium hydroxide (1 M) was added until ph˜9 was reached. The mixture was vigorously stirred for an hour after which the organic layer was decanted. The aqueous layer was extracted three times with ethyl acetate. The organic layers were collected, dried over magnesium sulphate and concentrated in vacuo. The crude mixture was purified by flash chromatography on silica gel (eluent gradient: pure dichloromethane to 6% methanol in dichloromethane). The title compound was obtained as a white solid. ¹H NMR (CDCl₃)=2.10 (s, 3H), 6.51 (d, 1H), 7.36 (d, 1H), 7.41-7.46 (m, 2H), 7.48-7.52 (m, 3H), 9.73 (s, 1H).

### d) Synthesis of 6-bromo-3-methyl-2-phenyl-pyridine

To a solution of 5-methyl-6-phenyl-1H-pyridin-2-one (1.6 g, 8.6 mmol) in toluene (35 mL) was added in one portion phosphorus oxybromide (5.0 g, 17.3 mmol). The mixture was refluxed for 2 hours, and then cooled to 0° C., covered with ethyl acetate and quenched with an aqueous solution of sodium hydroxide (2 M) at 0° C. The organic layer was decanted, dried and concentrated. The crude mixture was filtered over a pad of silica gel with a mixture of 25% ethyl acetate in cyclohexane. The title compound was obtained as a colourless oil. ¹H NMR (CDCl₃)=2.34 (s, 3H), 7.39 (d, 1H), 7.40-7.48 (m, 5H), 7.53 (d, 1H).

### e) Synthesis of 3-methyl-2-phenyl-6-tributylstannanyl-pyridine

In a dry flask, under argon, a solution of n-butyl lithium in tetrahydrofuran (1.5 M, 17 mL, 25.7 mmol) was added dropwise to a solution of 6-bromo-3-methyl-2-phenyl-pyridine (5.8 g, 23.4 mmol) in anhydrous tetrahydrofuran (100 mL), at −78° C. The solution was stirred at that temperature for 45 minutes, after which tributyltin chloride (6.4 mL, 23.4 mmol) was added dropwise, at −78° C. The solution was allowed to warm up to room temperature over an hour, before which a saturated aqueous solution of ammonium chloride was added. The organic layer was decanted. The aqueous layer was further extracted twice with ethyl acetate. The organic layers were collected, dried over magnesium sulphate and concentrated in vacuo. The title compound was obtained as a pale yellow oil. ¹H NMR (CDCl₃): 0.92 (m, 9H), 1.14 (m, 6H), 1.48 (m, 6H), 1.60 (m, 6H), 7.28 (d, 1H), 7.47-7.50 (m, 2H), 7.52-7.58 (m, 2H), 7.61 (m, 2H).

### f) Synthesis of 2-bromo-4-methylquinazoline

To a degassed mixture of 2,4-dibromoquinazoline (200 mg, 0.69 mmol), trimethylboroxine (0.10 mL, 0.69 mmol) and potassium carbonate (300 mg, 2.1 mmol) in anhydrous dioxane (2.5 mL) in a microwave vial was added tetrakis(triphenylphosphine)palladium(0) (80 mg, 69 μmol) under argon. The vial was sealed and irradiated in a microwave oven for 5 minutes at 150° C. The crude mixture was diluted with dichloromethane and washed with water. The organic layer was decanted, dried over magnesium sulphate and concentrated in vacuo. The crude mixture was purified by flash chromatography on silica gel (eluent gradient: 0% to 25% ethyl acetate in cyclohexane) to yield the title compound. ¹H NMR (CDCl₃): 2.96 (s, 3H), 7.58 (app. t, 1H), 7.93 (app. t, 1H), 7.98 (d, 1H), 8.10 (d, 1H).

### g) Synthesis of 4-Methyl-2-(5-methyl-6-phenyl-pyridin-2-yl)-quinazoline

To a degassed, stirred solution of 3-methyl-2-phenyl-6-tributylstannanyl-pyridine (247 mg, 0.54 mmol), 2-bromo-4-methylquinazoline (74 mg, 0.33 mmol) and lithium chloride (39 mg, 0.92 mmol) in anhydrous N,N-dimethylformamide (2 mL) in a supelco vial, was added tetrakis(triphenylphosphine)palladium(0) (38 mg, 33 μmol). The vial was sealed and heated to 100° C., overnight. The crude mixture was then diluted with acetonitrile and washed 3 times with hexane. The acetonitrile layer was concentrated in vacuo and taken up in ethyl acetate. It was washed 3 times with water, dried over magnesium sulphate and concentrated. The organic layer was decanted, dried and concentrated. The crude thus obtained was purified by flash chromatography on silica gel (eluent gradient: 0% to 30% ethyl acetate in cyclohexane). The title compound was obtained as a white solid. m.p.: 141-143° C. ¹H NMR (CDCl₃): 2.36 (s, 3H), 2.96 (s, 3H), 7.29-7.33 (m, 1H), 7.38 (app. t, 2H), 7.51 (t, 1H), 7.60 (d, 2H), 7.68 (d, 1H), 7.77 (t, 1H), 8.02 (d, 1H), 8.10 (d, 1H), 8.40 (d, 1H).

Throughout this description, temperatures are given in degrees Celsius and “m.p.” means melting point

### Conditions A

MS ZMD Mass Spectrometer from Waters (single quadrupole mass spectrometer), ionization method: electrospray, polarity: positive ionization, capillary (kV) 3.00, cone (V) 30.00, Extractor (V) 3.00, source temperature (° C.) 150, desolvation temperature (° C.) 320, cone gas flow (L/Hr) 50, desolvation gas flow (L/Hr) 400, mass range: 150 to 800 Da.
LC Alliance 2795 LC HPLC from Waters: quaternary pump, heated column compartment and diode-array detector.
Column: Waters Atlantis dc18; length: 20 mm; internal diameter: 3 mm; particle size: 3 μm, temperature (° C.) 40, DAD wavelength range (nm): 200 to 500, solvent gradient: A=0.1% of formic acid in water and B: 0.1% of formic acid in acetonitrile.

| Time (min) | A % | B % | Flow (ml/min) |
|---|---|---|---|
| 0.0 | 80 | 20 | 1.7 |
| 2.5 | 0.0 | 100 | 1.7 |
| 2.8 | 0.0 | 100 | 1.7 |
| 2.9 | 80 | 20 | 1.7 |

### Condition B

MS ZQ Mass Spectrometer from Waters (single quadrupole mass spectrometer), ionization method: electrospray, polarity: positive ionization, capillary (kV) 3.00, cone (V) 30.00, extractor (V) 3.00, source temperature (° C.) 100, desolvation temperature (° C.) 200, cone gas flow (L/Hr) 200, desolvation gas flow (L/Hr) 250, mass range: 150 to 800 Da.
LC 1100er Series HPLC from Agilent: quaternary pump, heated column compartment and diode-array detector.
Column: Waters Atlantis dc18; length: 20 mm; internal diameter: 3 mm; particle size: 3 μm, temperature (° C.) 40, DAD wavelength range (nm): 200 to 500, solvent gradient: A=0.1% of formic acid in water and B: 0.1% of formic acid in acetonitrile.

| Time (min) | A % | B % | Flow (ml/min) |
|---|---|---|---|
| 0.0 | 80 | 20 | 1.7 |
| 2.5 | 0.0 | 100 | 1.7 |
| 2.8 | 0.0 | 100 | 1.7 |
| 2.9 | 80 | 20 | 1.7 |

### Condition C

MS ACQUITY SQD Mass Spectrometer from Waters (Single quadrupole mass spectrometer)—Ionisation method: Electrospray—Polarity: positive ions—Capillary (kV) 3.00, Cone (V) 20.00, Extractor (V) 3.00, Source Temp (° C.) 150, Desolvation Temp (° C.) 400, Cone Gas Flow (L/Hr) 60, Desolvation Gas Flow (L/Hr) 700—Massrange: 100 to 800 Da—DAD Wavelength range (nm): 210 to 400.
LC Method Waters ACQUITY HPLC with the following HPLC gradient conditions (Solvent A: Water/Methanol 9:1, 0.1% formic acid and Solvent B: Acetonitrile, 0.1% formic acid)
Column: Waters ACQUITY HPLC HSS T3; Column length: 30 mm; Internal diameter of column: 2.1 mm; Particle Size: 1.8 micron; Temperature: 60° C.

| Time (minutes) | A (%) | B (%) | Flow rate (ml/min) |
|---|---|---|---|
| 0 | 80 | 20 | 1.5 |
| 0.1 | 75 | 25 | 1.5 |
| 0.2 | 70 | 30 | 0.75 |
| 1.20 | 0 | 100 | 0.75 |
| 1.40 | 0 | 100 | 0.75 |
| 1.45 | 80 | 20 | 0.75 |

### Conditions E

MS ZQ Mass Spectrometer from Waters (Single quadrupole mass spectrometer); Ionisation method: Electrospray; Polarity: positive ions; Capillary (kV) 3.00, Cone (V) 30.00, Extractor (V) 2.00, Source Temperature (° C.) 100, Desolvation Temperature (° C.) 250, Cone Gas Flow (L/Hr) 50, Desolvation Gas Flow (L/Hr) 400; Mass range: 150 to 1000 Da
LC HP 1100 HPLC from Agilent: solvent degasser, quaternary pump (ZCQ)/binary pump (ZDQ), heated column compartment and diode-array detector. Solvent Gradient: A=water+0.05% HCOOH, B=Acetonitril/Methanol (4:1, v:v)+0.04% HCOOH
Column: Phenomenex Gemini C18, 3 μm (micro meter) particle size, 110 Å (Ångström), 30×3 mm, Temp: 60° C.; DAD Wavelength range (nm): 200 to 500

| Time | A % | B % | Flow (ml/min) |
|---|---|---|---|
| 0.00 | 95.0 | 5.0 | 1.700 |
| 2.00 | 0.0 | 100.0 | 1.700 |
| 2.80 | 0.0 | 100.0 | 1.700 |
| 2.90 | 95.0 | 5.0 | 1.700 |
| 3.00 | 95.0 | 5.0 | 1.700 |

TABLE 2

| | | | (M + H)⁺ | LC/MS | Melting Point |
|---|---|---|---|---|---|
| Entry | Structure | RT (min) | (measured) | method | (C.) |
| 1 | 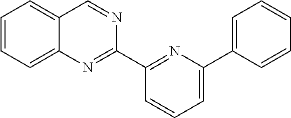 | 1.42 | 284.17 | A | |
| 2 | 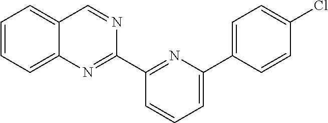 | 1.66 | 318.09 | A | |
| 3 | 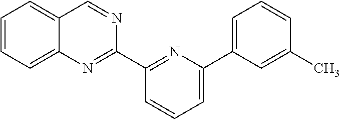 | 1.56 | 298.17 | A | |
| 4 | 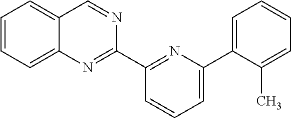 | 1.43 | 298.17 | A | |
| 5 | 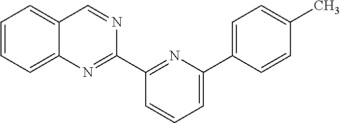 | 1.55 | 298.17 | A | |
| 6 | 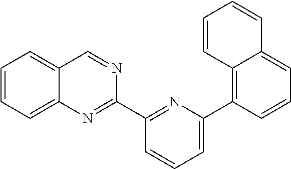 | 1.66 | 334.11 | A | |
| 7 | 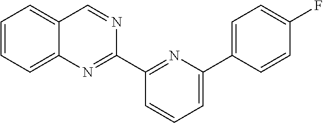 | 1.5 | 302.13 | A | |
| 8 | 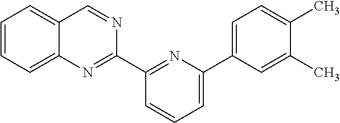 | 1.64 | 312.16 | A | |
| 9 | 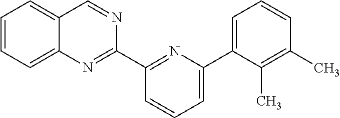 | 1.5 | 312.16 | A | |
| 10 | 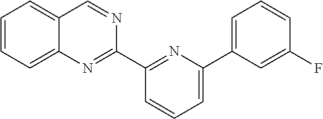 | 1.51 | 302.14 | A | |
| 11 | 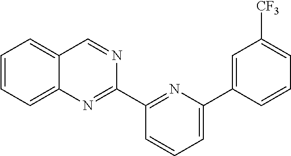 | 1.76 | 352.32 | A | |
| 12 | 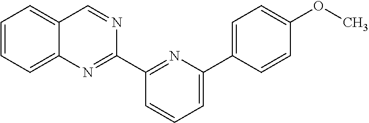 | 1.39 | 314.14 | A | |
| 13 | 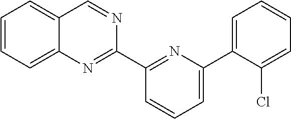 | 1.51 | 318.09 | A | |
| 14 | 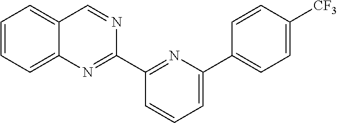 | 1.76 | 352.13 | A | |
| 15 | 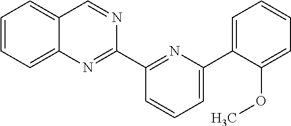 | 0.91 | 314.14 | A | |
| 16 | 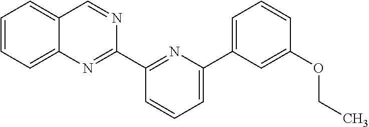 | 1.59 | 328.13 | A | |
| 17 | 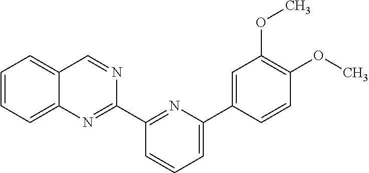 | 1.26 | 344.31 | A | |
| 18 | 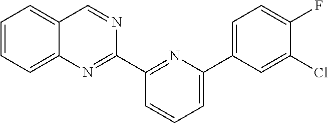 | 1.72 | 336.05 | A | |
| 19 | 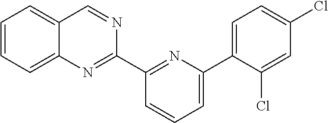 | 1.77 | 351.99 | A | |
| 20 | 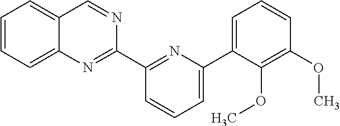 | 1.14 | 344.41 | A | |
| 21 | 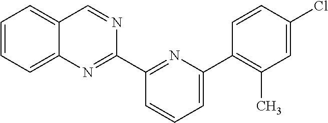 | 1.72 | 332.08 | A | |
| 22 | 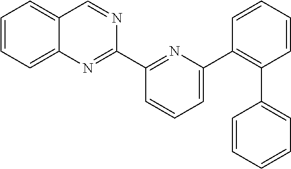 | 1.69 | 360.40 | A | |
| 23 | 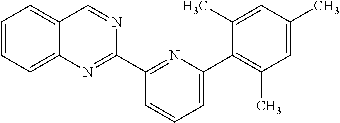 | 1.57 | 326.33 | A | |
| 24 | 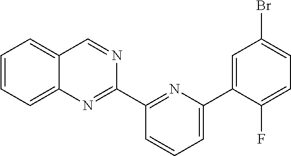 | 1.76 | 379.96 | A | |
| 25 | 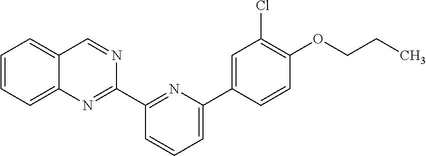 | 1.95 | 376.07 | A | |
| 26 | 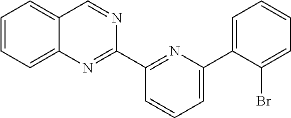 | 1.56 | 363.96 | A | |
| 27 | 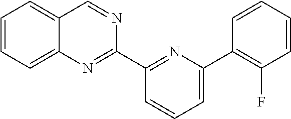 | 1.46 | 302.13 | A | |
| 28 | 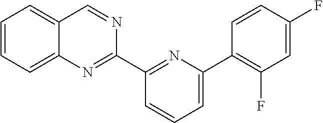 | 1.55 | 320.17 | A | |
| 29 | 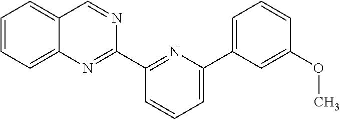 | 1.46 | 314.19 | A | |
| 30 | 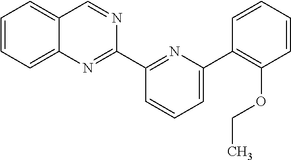 | 0.99 | 328.35 | A | |
| 31 | 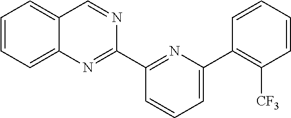 | 1.59 | 352.30 | A | |
| 32 | 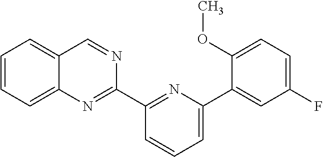 | 1.28 | 332.31 | A | |
| 33 | 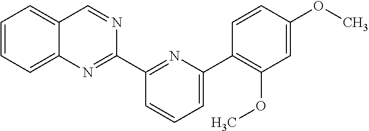 | 0.95 | 344.11 | A | |
| 34 | 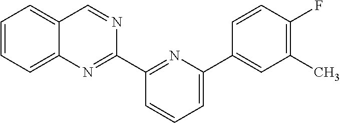 | 1.62 | 316.26 | A | |
| 35 | 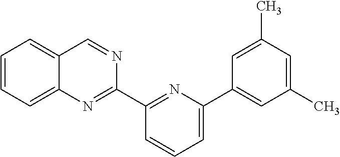 | 1.71 | 312.16 | A | |
| 36 | 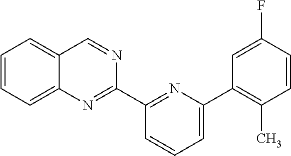 | 1.56 | 316.19 | A | |
| 37 | 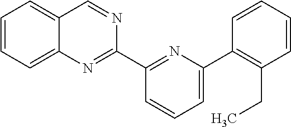 | 1.6 | 312.16 | A | |
| 38 | 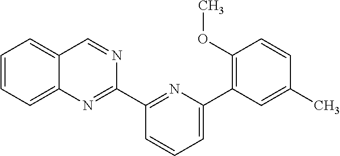 | 1.14 | 328.27 | A | |
| 39 | 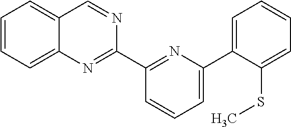 | 1.48 | 330.09 | A | |
| 40 | 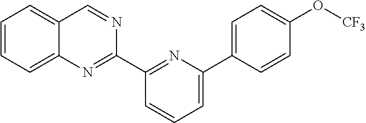 | 1.79 | 368.17 | A | |
| 41 | 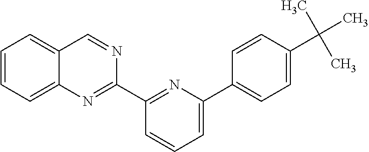 | 1.99 | 340.40 | A | |
| 42 | 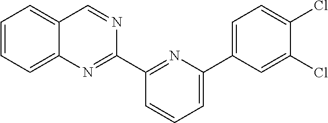 | 1.87 | 351.99 | A | |
| 43 | 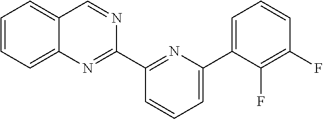 | 1.56 | 320.10 | A | |
| 44 | 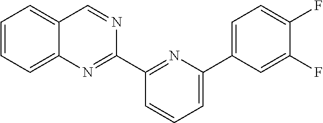 | 1.59 | 320.24 | A | |
| 45 | 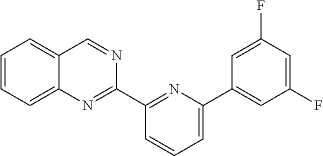 | 1.62 | 320.10 | A | |
| 46 | 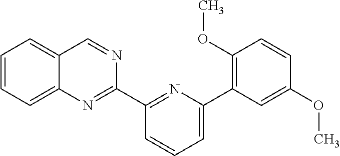 | 1.06 | 344.10 | A | |
| 47 | 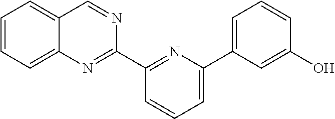 | 1.1 | 300.13 | A | |
| 48 | 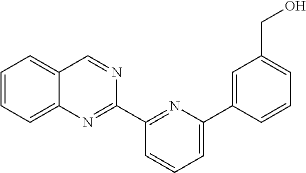 | 1.02 | 314.13 | A | |
| 49 | 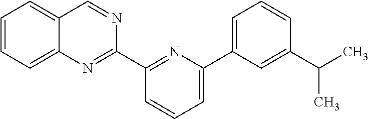 | 1.83 | 326.20 | A | |
| 50 | 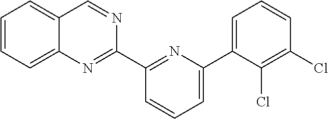 | 1.69 | 352.00 | A | |
| 51 | 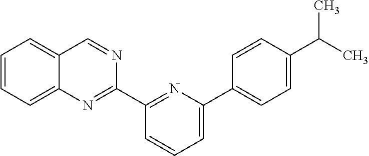 | 1.82 | 326.25 | A | |
| 52 | 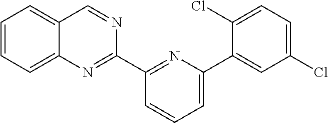 | 1.77 | 352.00 | A | |
| 53 | 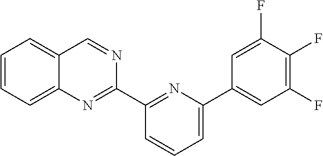 | 1.69 | 338.20 | A | |
| 54 | 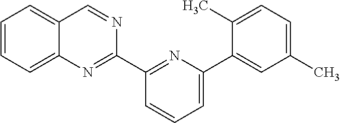 | 1.58 | 312.15 | A | 120-122 |
| 55 | 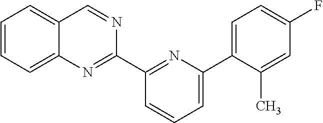 | 1.52 | 316.12 | A | 128-131 |
| 56 | 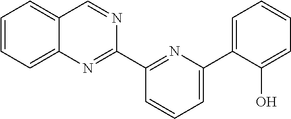 | 1.7 | 300.13 | A | |
| 57 | 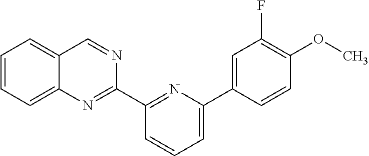 | 1.49 | 332.10 | A | |
| 58 | 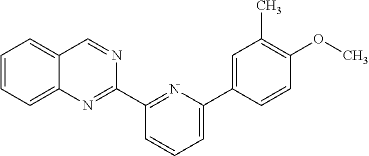 | 1.52 | 328.37 | A | |
| 59 | 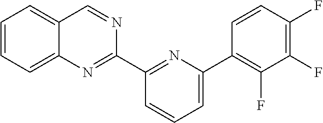 | 1.65 | 338.06 | A | |
| 60 | 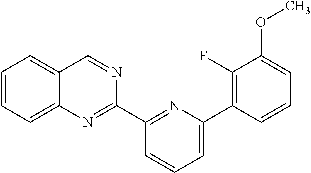 | 1.46 | 332.09 | A | 142-143 |
| 61 | 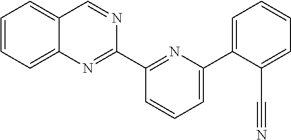 | 1.26 | 309.12 | A | |
| 62 | 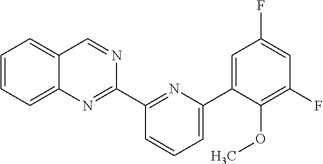 | 1.6 | 350.24 | A | |
| 63 | 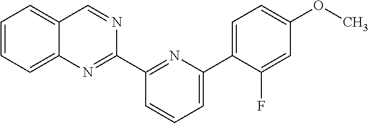 | 1.52 | 332.10 | A | |
| 64 | 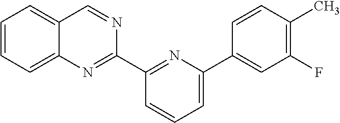 | 1.66 | 316.12 | A | |
| 65 | 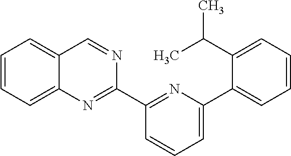 | 1.7 | 326.39 | A | |
| 66 | 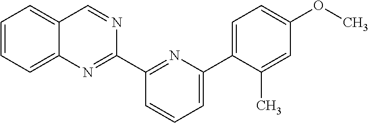 | 1.32 | 328.37 | A | |
| 67 | 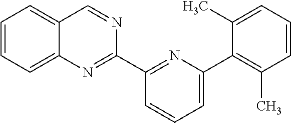 | 1.47 | 312.21 | A | |
| 68 | 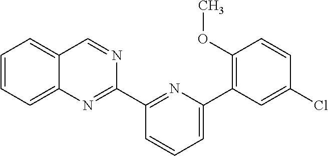 | 1.46 | 348.22 | A | |
| 69 | 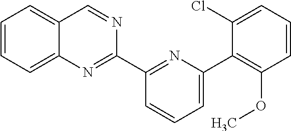 | 1.43 | 348.05 | A | |
| 70 | 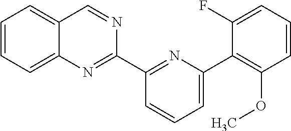 | 1.25 | 332.27 | A | |
| 71 | 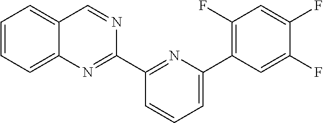 | 1.67 | 338.06 | A | |
| 72 | 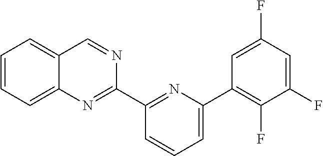 | 1.67 | 338.06 | A | |
| 73 | 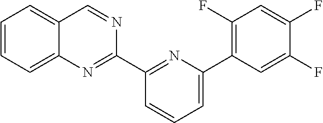 | 1.65 | 326.30 | A | |
| 74 | 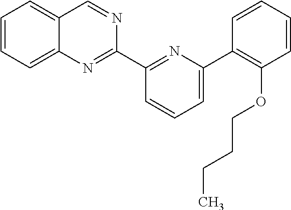 | 1.34 | 356.42 | A | |
| 75 | 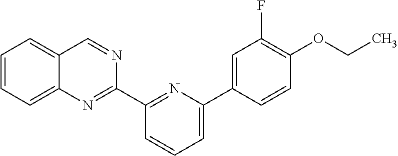 | 1.64 | 346.10 | A | |
| 76 | 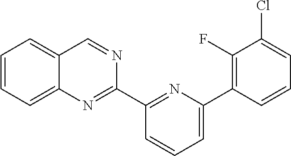 | 1.71 | 336.05 | A | |
| 77 | 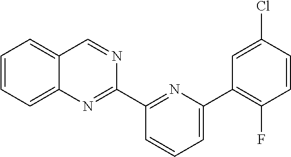 | 1.73 | 336.04 | A | |
| 78 | 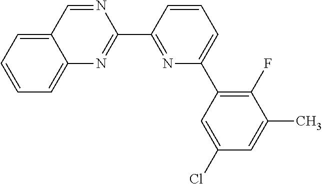 | 1.89 | 350.15 | A | |
| 79 | 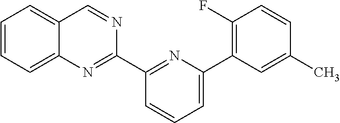 | 1.65 | 316.11 | A | |
| 80 | 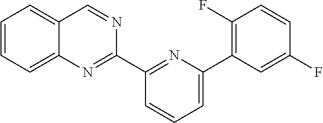 | 1.59 | 320.32 | A | |
| 81 | 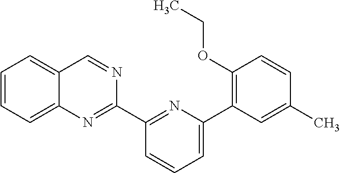 | 1.18 | 342.49 | A | |
| 82 | 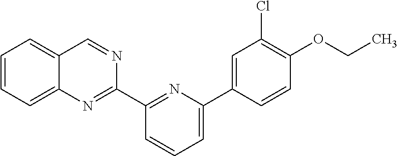 | 1.78 | 362.05 | A | |
| 83 | 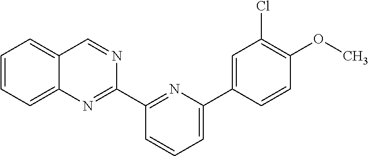 | 1.61 | 348.05 | A | |
| 84 | 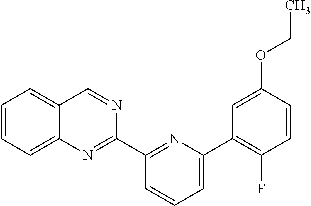 | 1.69 | 346.35 | A | |
| 85 | 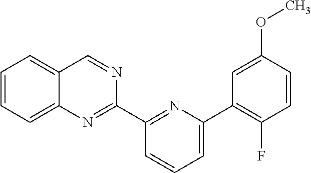 | 1.53 | 332.29 | A | |
| 86 | 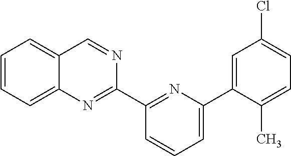 | 1.76 | 332.06 | A | |
| 87 | 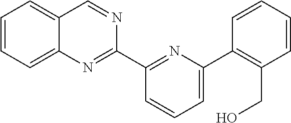 | 1.36 | 314.28 | A | |
| 88 | 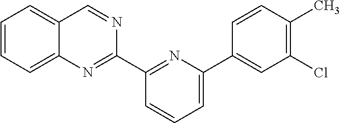 | 1.82 | 332.07 | A | |
| 89 | 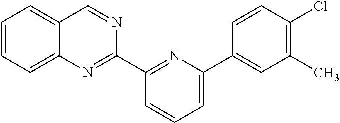 | 1.81 | 332.07 | A | |
| 90 | 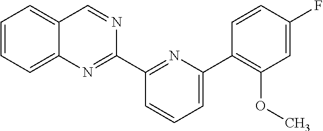 | 1.17 | 332.29 | A | 183-185 |
| 91 | 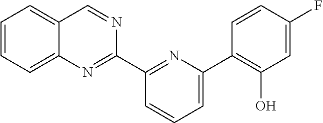 | 1.84 | 318.10 | A | |
| 92 | 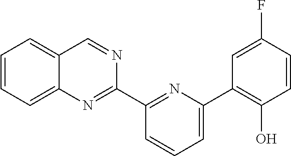 | 1.82 | 318.10 | A | |
| 93 | 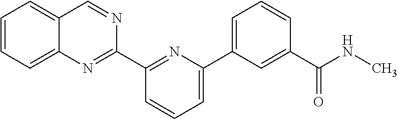 | 1.17 | 341.17 | A | |
| 94 | 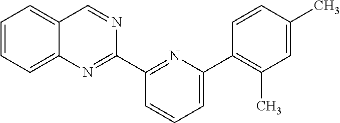 | 1.53 | 312.32 | A | |
| 95 | 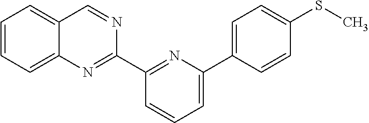 | 1.62 | 330.21 | A | |
| 96 | 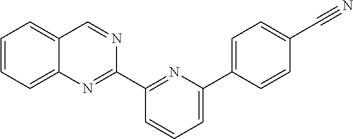 | 1.42 | 309.19 | A | |
| 97 | 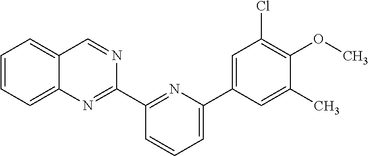 | 1.79 | 362.10 | A | |
| 98 | 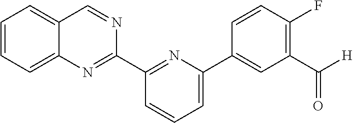 | 1.4 | 330.18 | A | |
| 99 | 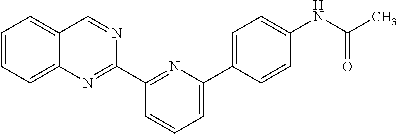 | 1.03 | 341.10 | A | |
| 100 | 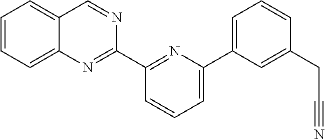 | 1.34 | 323.11 | A | |
| 101 | 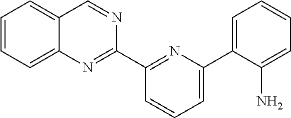 | 0.68 | 299.19 | C | |
| 102 | 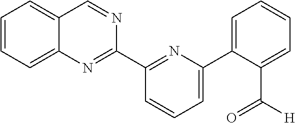 | 0.44 | 312.06 | C | |
| 103 | 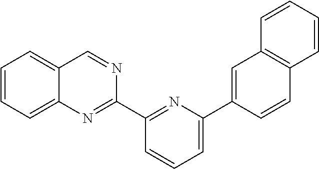 | 1.02 | 334.2 | C | |
| 104 | 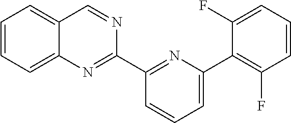 | 0.83 | 320.17 | C | |
| 105 | 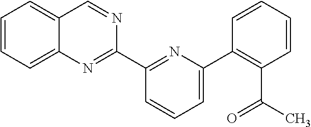 | 0.74 | 326.19 | C | |
| 106 | 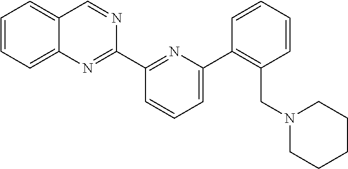 | 0.59 | 381.27 | C | |
| 107 | 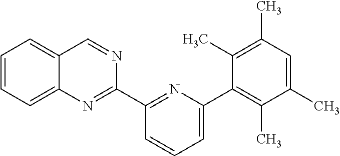 | 1.05 | 340.26 | C | |
| 108 | 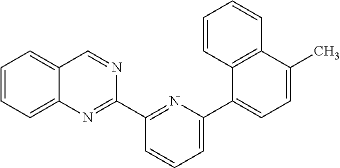 | 1.05 | 348.22 | C | |
| 109 | 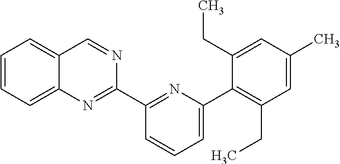 | 1.16 | 354.27 | C | |
| 110 | 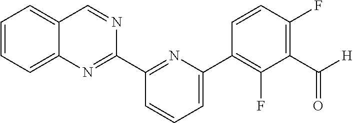 | 0.8 | 348.3 | C | |
| 111 | 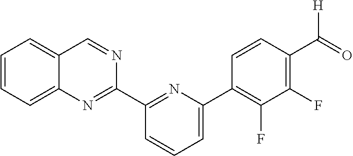 | 0.86 | 348.11 | C | |
| 112 | 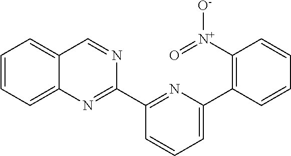 | 0.76 | 329.17 | C | |
| 113 | 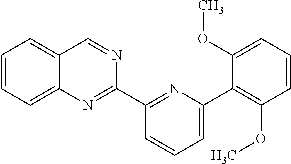 | 0.55 | 344.1 | C | |
| 114 | 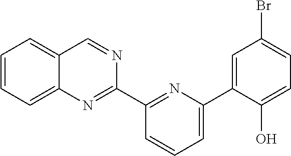 | 1.15 | 378.03 | C | |
| 115 | 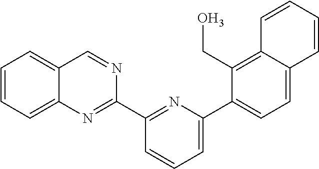 | 0.99 | 364.16 | C | |
| 116 | 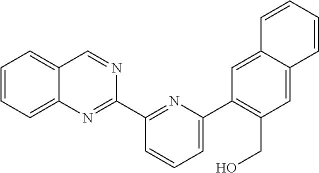 | 0.98 | 364.21 | C | |
| 117 | 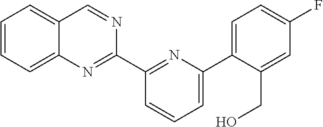 | 0.84 | 332.2 | C | |
| 118 | 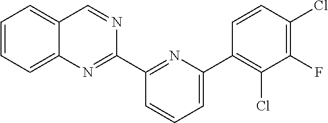 | 1.04 | 370.09 | C | |
| 119 | 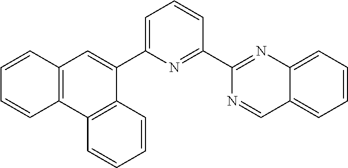 | 1.12 | 384.22 | C | |
| 121 | 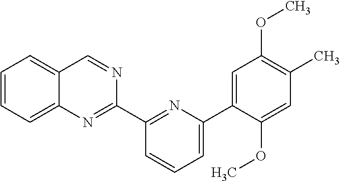 | 0.69 | 358.22 | C | |
| 122 | 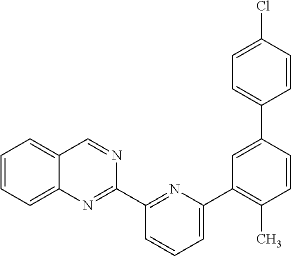 | 1.23 | 408.2 | C | |
| 123 | 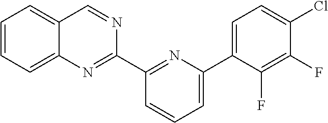 | 1.05 | 354.11 | C | |
| 124 | 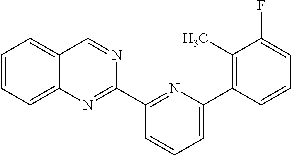 | 0.94 | 316.2 | C | |
| 125 | 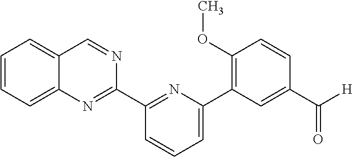 | 0.69 | 342.21 | C | |
| 126 | 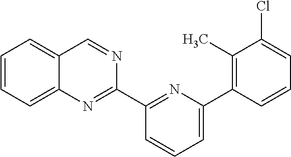 | 1.02 | 332.17 | C | |
| 127 | 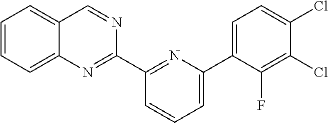 | 1.11 | 370.1 | C | |
| 128 | 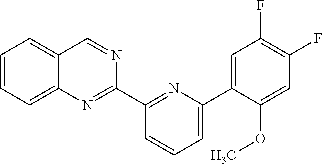 | 0.9 | 350.16 | C | |
| 129 | 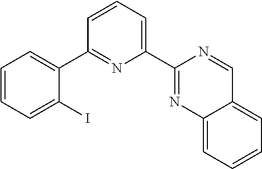 | | | | 164-165 |
| 130 | 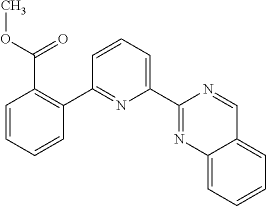 | | | | 155-158 |
| 131 | 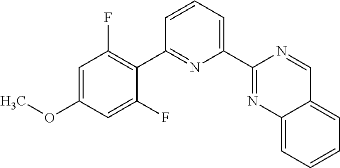 | | | | 142-144 |
| 132 | 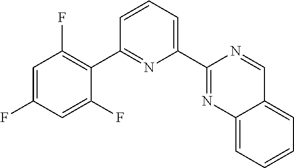 | 1.82 | 338 | E | |
| 133 | 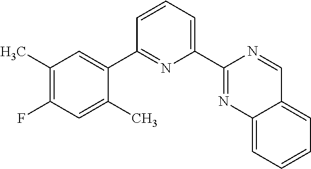 | | | | 138-140 |
| 134 | 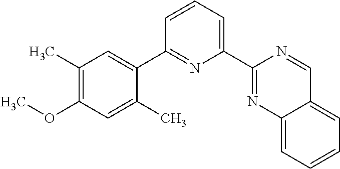 | | | | 150-152 |

Table 2 shows retention time and (M+H)⁺ value and/or melting point value measured for selected compounds of the formula I.a where R¹ is H and A is optionally substituted aryl

TABLE 3

| | | RT | (M + H)⁺ | | Melting |
|---|---|---|---|---|---|
| Entry | Structure | (min.) | (measured) | LC/MS | Point ° C. |
| 1 | 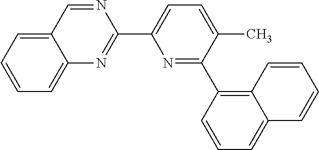 | 1.78 | 348.07 | B | |
| 2 | 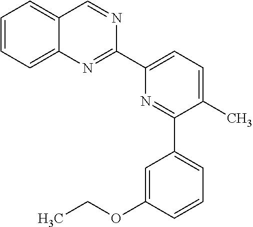 | 1.68 | 342.09 | B | |
| 3 | 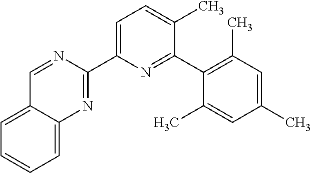 | 1.69 | 340.12 | B | |
| 4 | 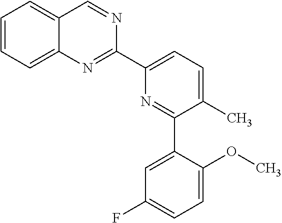 | 1.56 | 346.05 | B | |
| 5 | 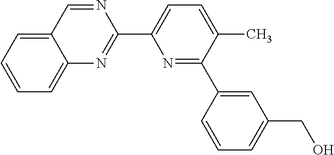 | 1.13 | 328.13 | B | |
| 6 | 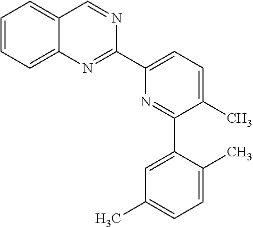 | 1.66 | 326.12 | B | |
| 7 | 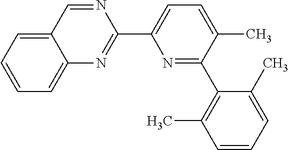 | 1.61 | 326.12 | B | |
| 8 | 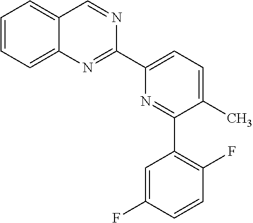 | 1.72 | 334.08 | B | |
| 9 | 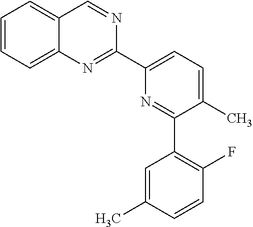 | 1.81 | 330.08 | B | |
| 10 | 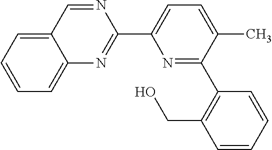 | 1.41 | 328.12 | B | |
| 11 | 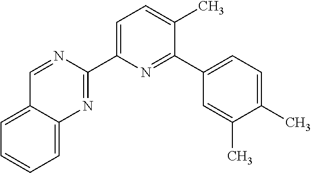 | 1.67 | 326.12 | B | |
| 12 | 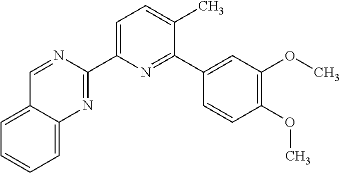 | 1.3 | 358.09 | B | |
| 13 | 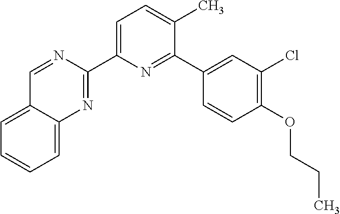 | 2.07 | 390.07 | B | |
| 14 | 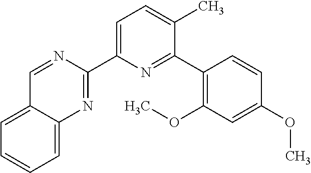 | 1.35 | 358.13 | B | |
| 15 | 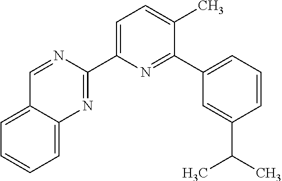 | 1.92 | 340.15 | B | |
| 16 | 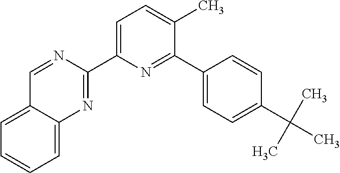 | 2.01 | 354.16 | B | |
| 17 | 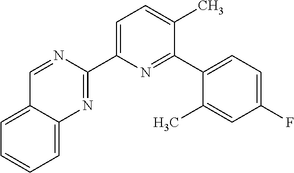 | 1.64 | 330.09 | B | |
| 18 | 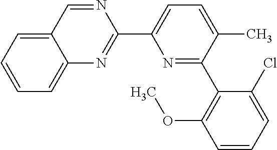 | 1.69 | 362.05 | B | |
| 19 | 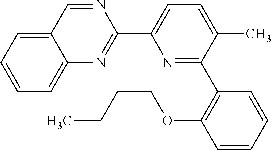 | 1.74 | 370.14 | B | |
| 20 | 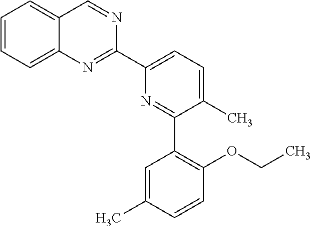 | 1.6 | 356.11 | B | |
| 21 | 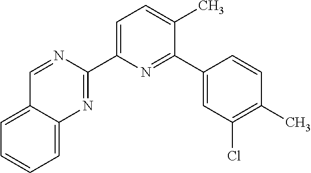 | 1.98 | 346.05 | B | |
| 22 | 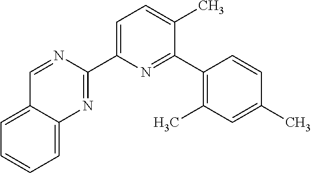 | 1.61 | 326.12 | B | |
| 23 | 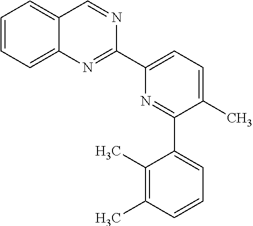 | 1.6 | 326.12 | B | |
| 24 | 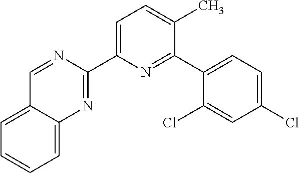 | 1.97 | 365.98 | B | |
| 25 | 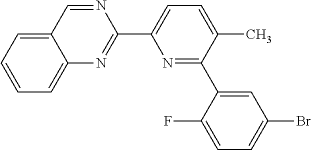 | 1.92 | 395.98 | B | |
| 26 | 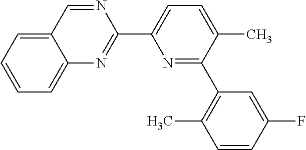 | 1.73 | 330.09 | B | |
| 27 | 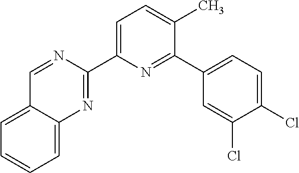 | 2.02 | 365.98 | B | |
| 28 | 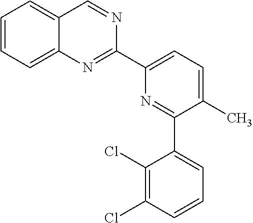 | 1.92 | 365.98 | B | |
| 29 | 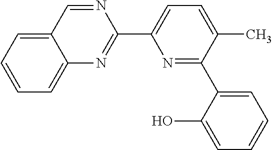 | 1.43 | 314.12 | B | |
| 30 | 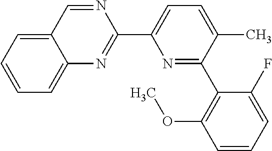 | 1.55 | 346.12 | B | |
| 31 | 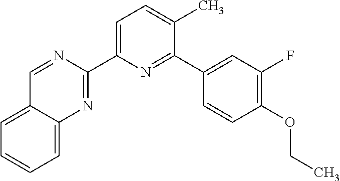 | 1.76 | 360.10 | B | |
| 32 | 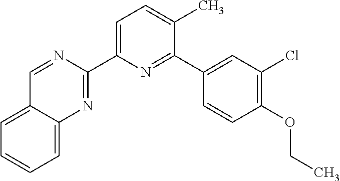 | 1.9 | 376.05 | B | |
| 33 | 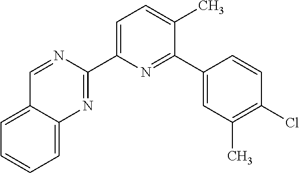 | 1.93 | 346.05 | B | |
| 34 | 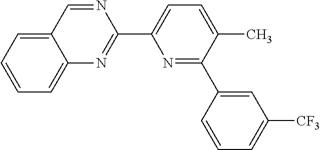 | 1.93 | 366.05 | B | |
| 35 | 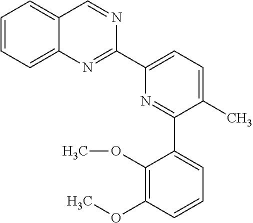 | 1.43 | 358.13 | B | |
| 36 | 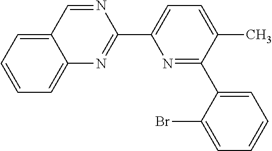 | 1.77 | 375.98 | B | |
| 37 | 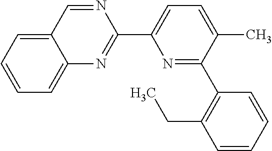 | 1.7 | 326.12 | B | |
| 38 | 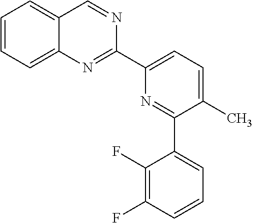 | 1.73 | 334.11 | B | |
| 39 | 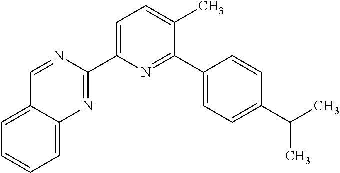 | 1.9 | 340.15 | B | |
| 40 | 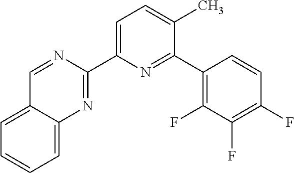 | 1.79 | 352.09 | B | |
| 41 | 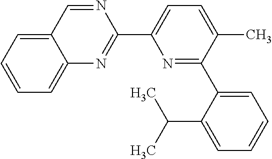 | 1.84 | 340.15 | B | |
| 42 | 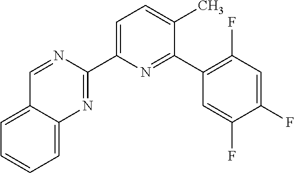 | 1.79 | 352.09 | B | |
| 43 | 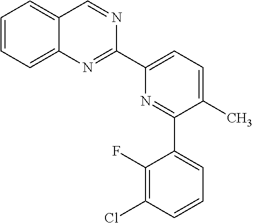 | 1.85 | 350.07 | B | |
| 44 | 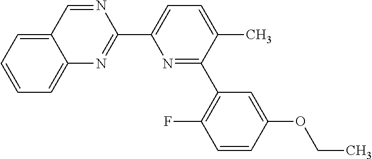 | 1.82 | 360.13 | B | |
| 45 | 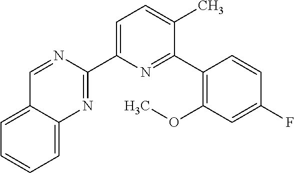 | 1.48 | 346.12 | B | |
| 46 | 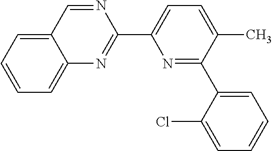 | 1.73 | 332.09 | B | |
| 47 | 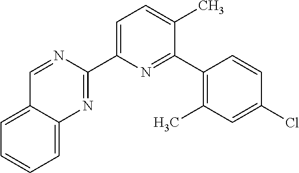 | 1.86 | 346.05 | B | |
| 48 | 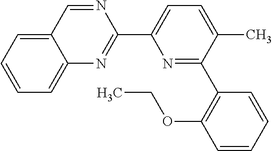 | 1.45 | 342.16 | B | |
| 49 | 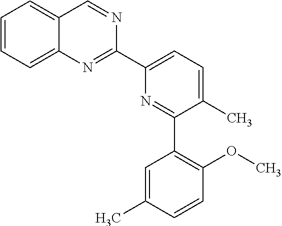 | 1.49 | 342.15 | B | |
| 50 | 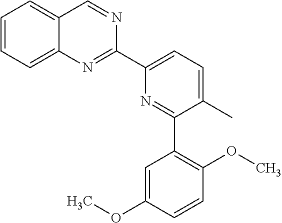 | 1.42 | 358.13 | B | |
| 51 | 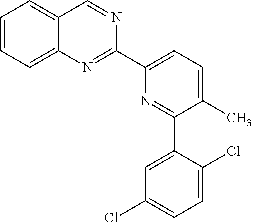 | 1.96 | 365.99 | B | |
| 52 | 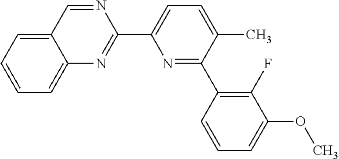 | 1.62 | 346.12 | B | |
| 53 | 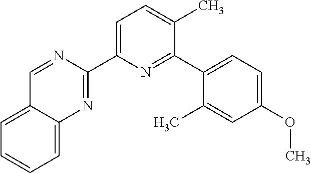 | 1.42 | 342.16 | B | |
| 54 | 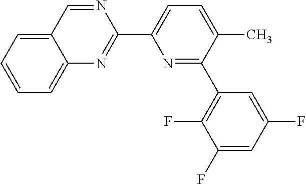 | 1.8 | 352.09 | B | |
| 55 | 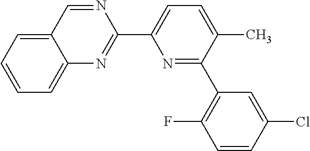 | 1.87 | 350.08 | B | |
| 56 | 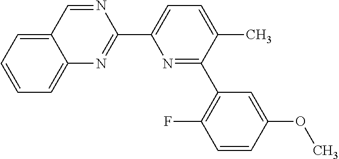 | 1.69 | 346.12 | B | |
| 57 | 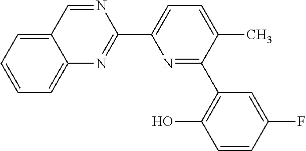 | 1.67 | 332.10 | B | |
| 58 | 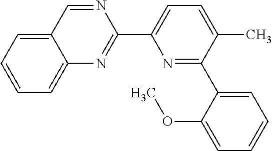 | 1.35 | 328.14 | B | |
| 59 | 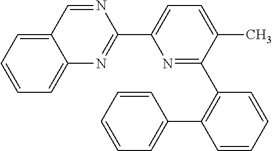 | 1.82 | 3374.10 | B | |
| 60 | 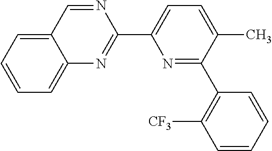 | 1.8 | 366.05 | B | |
| 61 | 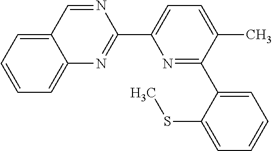 | 1.65 | 344.11 | B | |
| 62 | 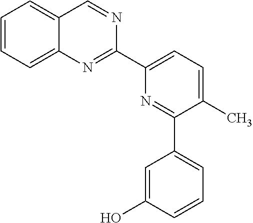 | 1.17 | 314.12 | B | |
| 63 | 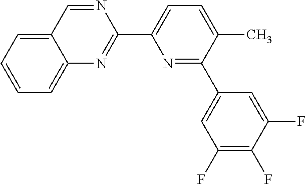 | 1.86 | 352.07 | B | |
| 64 | 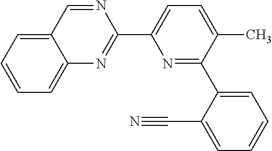 | 1.48 | 323.09 | B | |
| 65 | 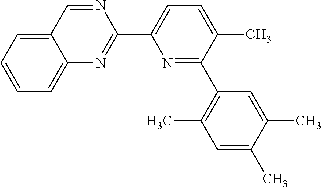 | 1.71 | 340.15 | B | |
| 66 | 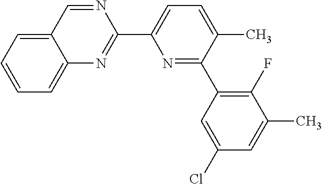 | 2.01 | 364.04 | B | |
| 67 | 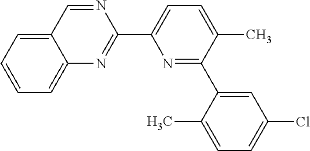 | 1.89 | 346.05 | B | |
| 68 | 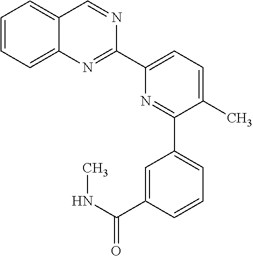 | 1.22 | 355.09 | B | |
| 69 | 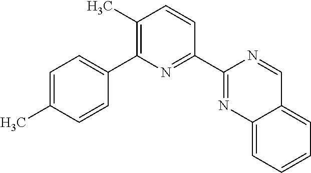 | | | | 182-183 |
| 70 | 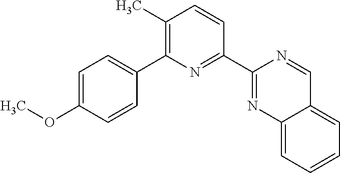 | | | | 176-177 |
| 71 | 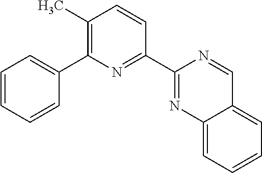 | | | | 191-193 |
| 72 | 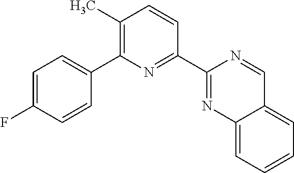 | | | | 183-186 |
| 73 | 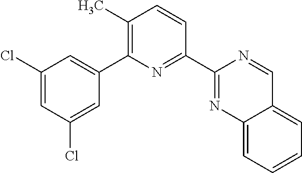 | | | | oil |
| 74 | 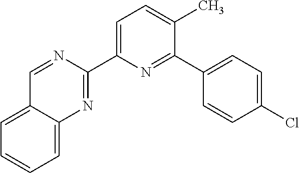 | 1.56 | 332.80 | B | oil |
| 75 | 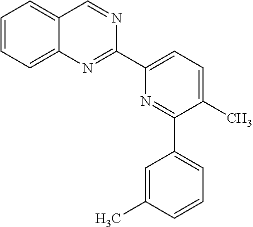 | 1.38 | 312.40 | B | oil |
| 76 | 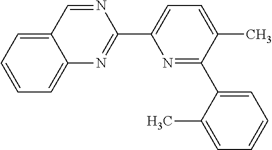 | 1.28 | 312.40 | B | 143-144 |
| 77 | 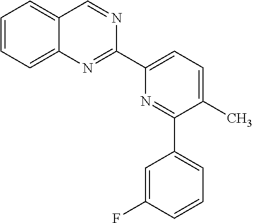 | 1.44 | 316.40 | B | oil |
| 78 | 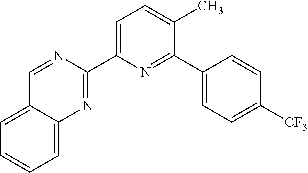 | 1.7 | 366.40 | B | |
| 79 | 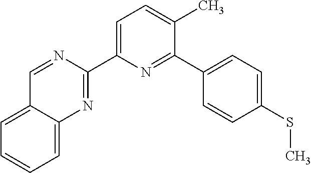 | 1.46 | 344.50 | B | |
| 80 | 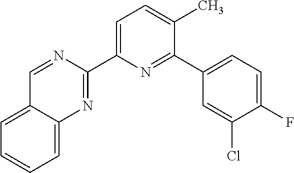 | 1.74 | 350.80 | B | |
| 81 | 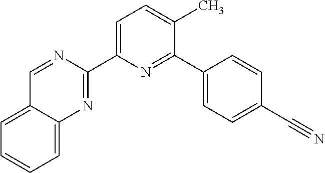 | 1.41 | 323.40 | B | |
| 82 | 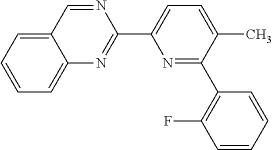 | 1.41 | 316.40 | B | oil |
| 83 | 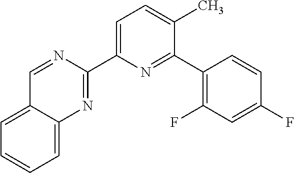 | 1.49 | 334.30 | B | |
| 84 | 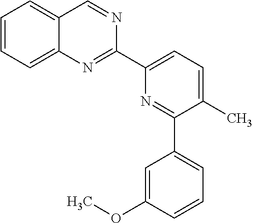 | 1.32 | 328.40 | B | oil |
| 85 | 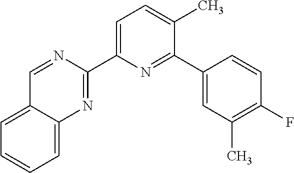 | 1.48 | 330.40 | B | 137-139 |
| 86 | 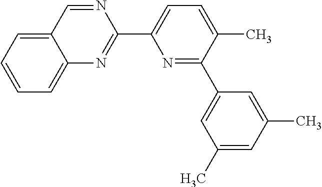 | 1.51 | 326.40 | B | oil |
| 87 | 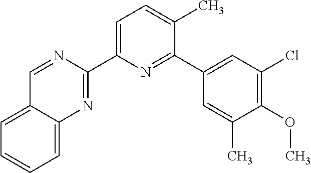 | 1.69 | 376.90 | B | |
| 88 | 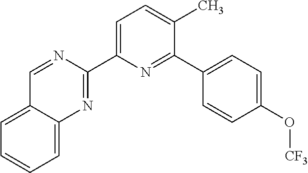 | 1.82 | 382.40 | B | |
| 89 | 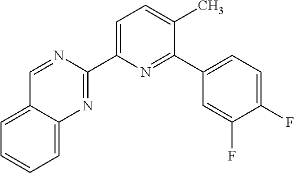 | 1.52 | 334.30 | B | 158-160 |
| 90 | 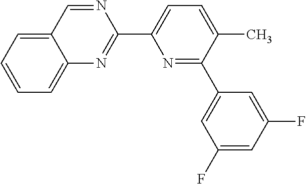 | 1.56 | 334.30 | B | 141-143 |
| 91 | 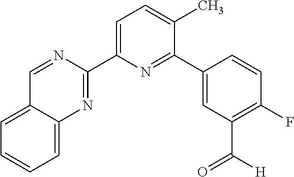 | 1.34 | 344.40 | B | |
| 92 | 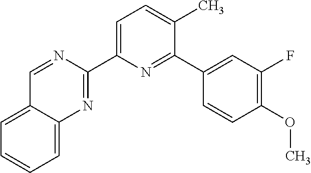 | 1.39 | 346.40 | B | 173-174 |
| 93 | 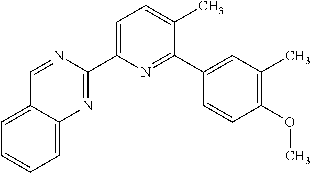 | 1.34 | 342.40 | B | oil |
| 94 | 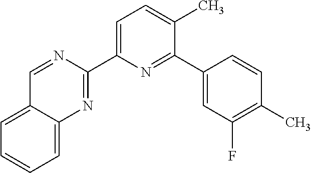 | 1.54 | 330.40 | B | oil |
| 95 | 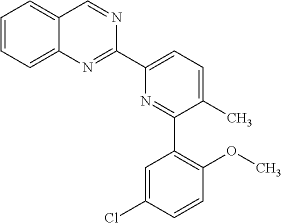 | 1.52 | 362.80 | B | |
| 96 | 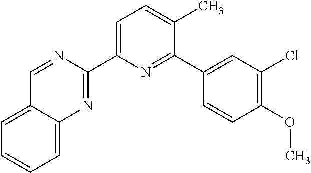 | 1.51 | 362.80 | B | |
| 97 | 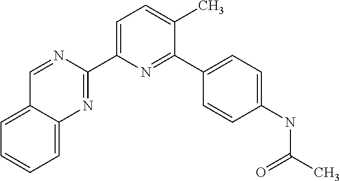 | 0.95 | 355.40 | B | |
| 98 | 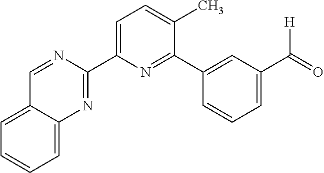 | 1.74 | 326.40 | B | |
| 99 | 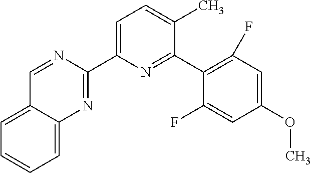 | 1.66 | 364.40 | B | |
| 100 | 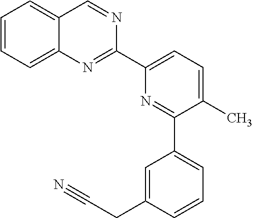 | 1.26 | 337.40 | B | |
| 101 | 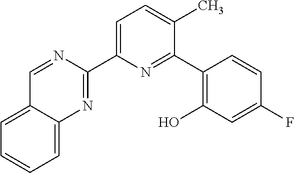 | 1.46 | 332.40 | B | |
| 102 | 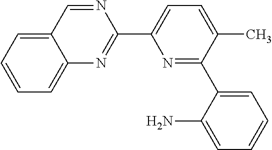 | 0.57 | 313.27 | C | |
| 103 | 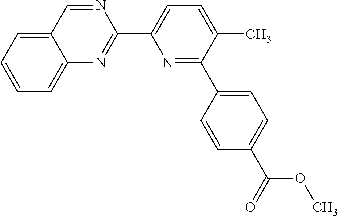 | 0.89 | 356.26 | C | |
| 104 | 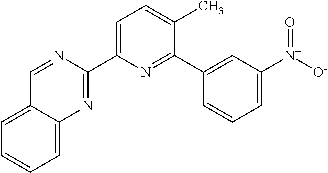 | 0.89 | 343.24 | C | |
| 105 | 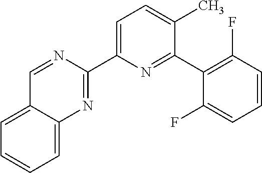 | 0.9 | 334.25 | C | |
| 106 | 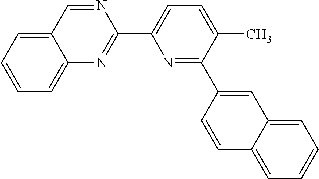 | 1.01 | 348.28 | C | |
| 107 | 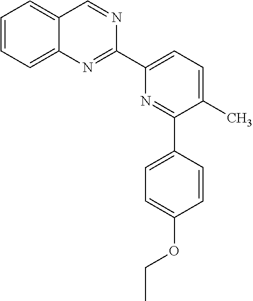 | 0.88 | 342.27 | C | |
| 108 | 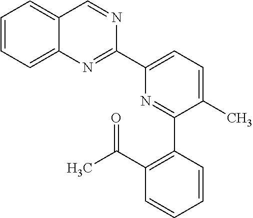 | 0.67 | 340.25 | C | |
| 109 | 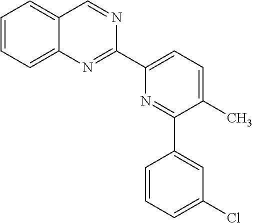 | 1 | 332.23 | C | |
| 110 | 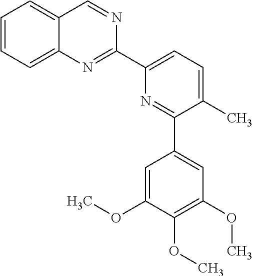 | 0.79 | 388.3 | C | |
| 111 | 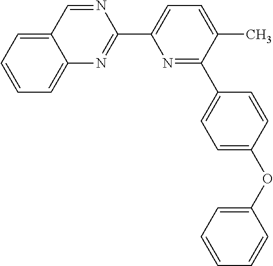 | 1.09 | 390.29 | C | |
| 112 | 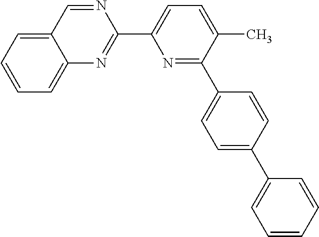 | 1.1 | 374.3 | C | |
| 113 | 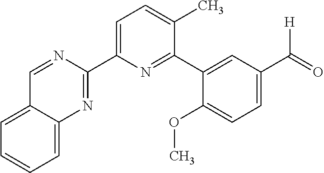 | 0.72 | 356.25 | C | |
| 114 | 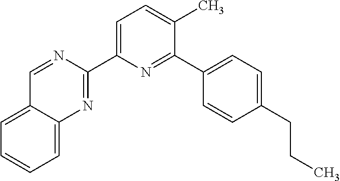 | 1.08 | 340.31 | C | |
| 115 | 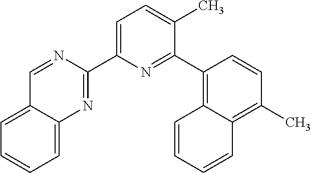 | 1.04 | 362.3 | C | |
| 116 | 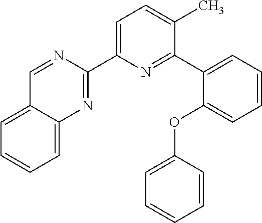 | 1.03 | 390.28 | C | |
| 117 | 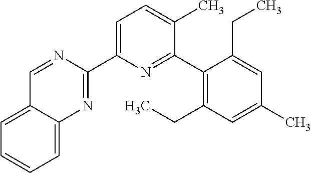 | 1.17 | 368.35 | C | |
| 118 | 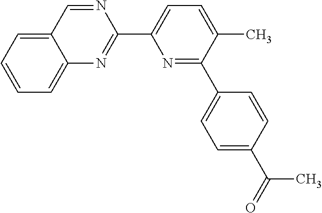 | 0.81 | 340.28 | C | |
| 119 | 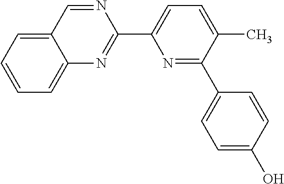 | 0.51 | 314.28 | C | |
| 120 | 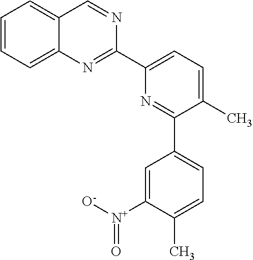 | 0.96 | 357.28 | C | |
| 121 | 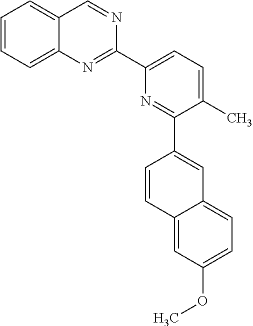 | 0.99 | 378.3 | C | |
| 122 | 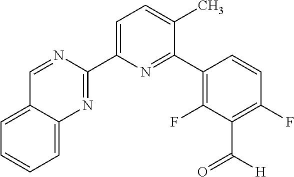 | 0.81 | 362.11 | C | |
| 123 | 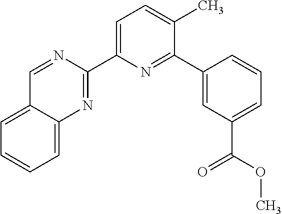 | 0.88 | 356.27 | C | |
| 124 | 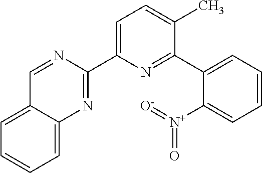 | 0.81 | 343.22 | C | |
| 125 | 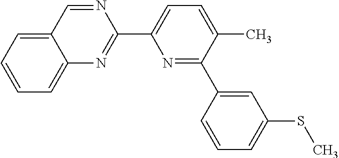 | 0.96 | 344.27 | C | |
| 126 | 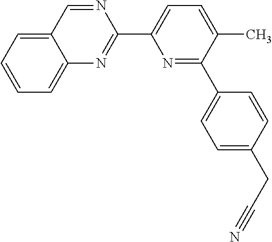 | 0.76 | 337.28 | C | |
| 127 | 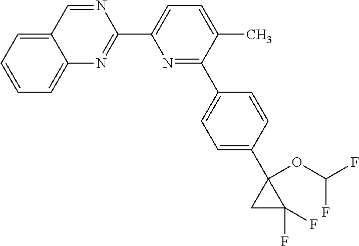 | 1.02 | 440.29 | C | |
| 128 | 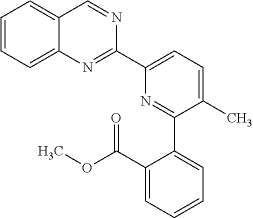 | 0.75 | 356.28 | C | |
| 129 | 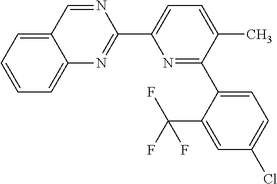 | 1.08 | 400.22 | C | |
| 130 | 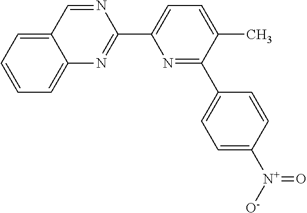 | 0.9 | 343.26 | C | |
| 131 | 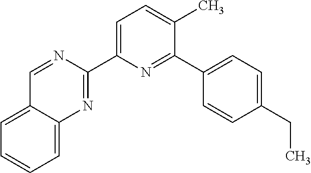 | 0.99 | 326.31 | C | |
| 132 | 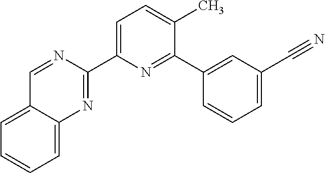 | 0.82 | 323.32 | C | |
| 133 | 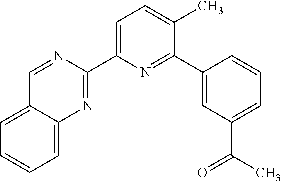 | 0.8 | 340.29 | C | |
| 134 | 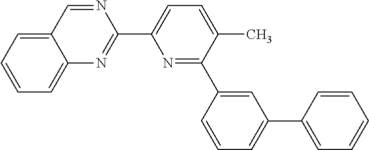 | 1.09 | 374.31 | C | |
| 135 | 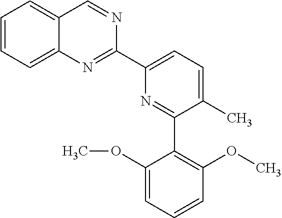 | 0.63 | 358.27 | C | |
| 136 | 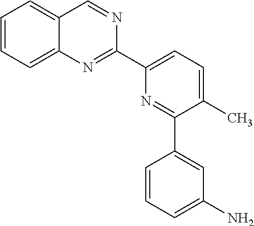 | 0.44 | 313.29 | C | |
| 137 | 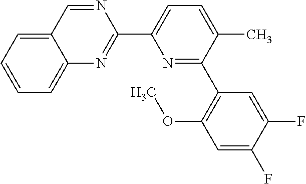 | 0.9 | 364.27 | C | |
| 138 | 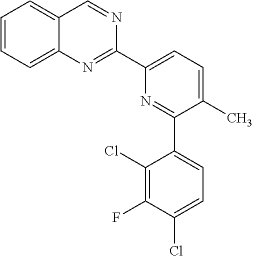 | 1.07 | 384.18 | C | |
| 139 | 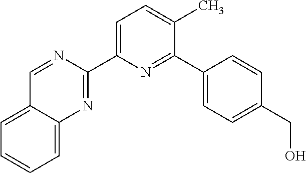 | 0.58 | 328.29 | C | |
| 140 | 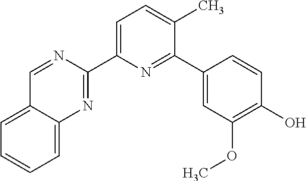 | 0.57 | 344.24 | C | |
| 141 | 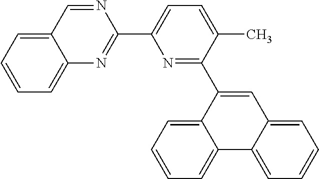 | 1.13 | 398.31 | C | |
| 142 | 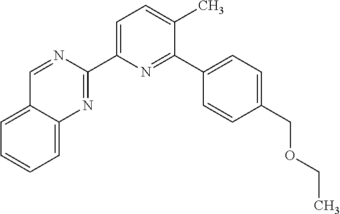 | 0.92 | 356.33 | C | |

Table 3 shows retention time and (M+H)⁺ value and/or melting point value measured for selected compounds of the formula I.a where R¹ is Methyl and A is optionally substituted aryl

TABLE 4

| | | RT | (M + H)⁺ | | MP |
|---|---|---|---|---|---|
| Entry | Structure | (min) | (measured) | LC/MS | ° C. |
| 1 | 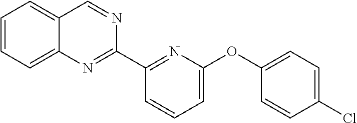 | 1.66 | 334.07 | A | |
| 2 | 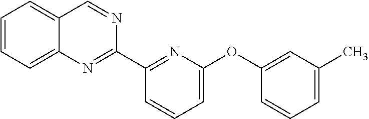 | 1.6 | 314.14 | A | |
| 3 | 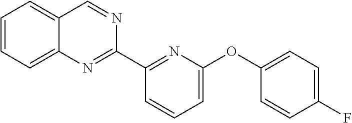 | 1.5 | 318.11 | A | |
| 4 | 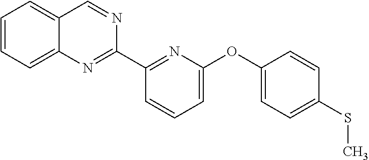 | 1.64 | 346.08 | A | |
| 5 | 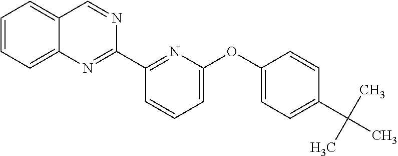 | 1.95 | 356.42 | A | |
| 6 | 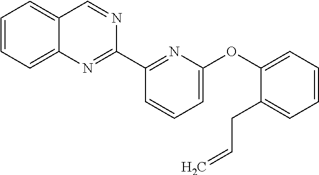 | 1.74 | 340.30 | A | |
| 7 | 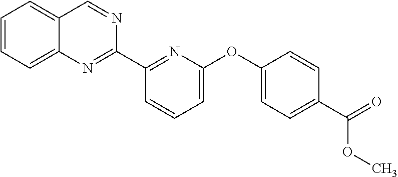 | 1.51 | 358.08 | A | |
| 8 | 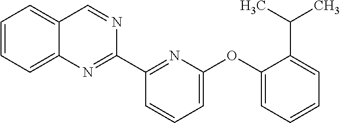 | 1.83 | 342.38 | A | |
| 9 | 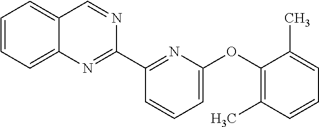 | 1.67 | 328.26 | A | |
| 10 | 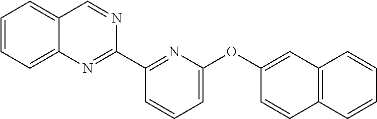 | 1.73 | 350.38 | A | |
| 11 | 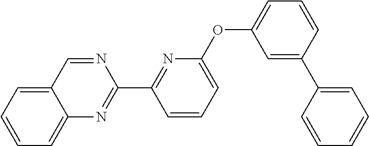 | 1.93 | 376.14 | A | |
| 12 | 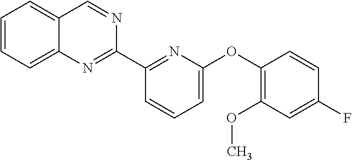 | 1.47 | 348.35 | A | |
| 13 | 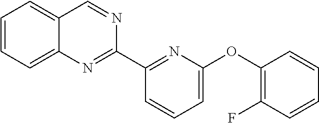 | 1.48 | 318.17 | A | |
| 14 | 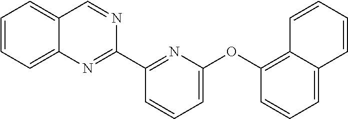 | 1.71 | 350.22 | A | |
| 15 | 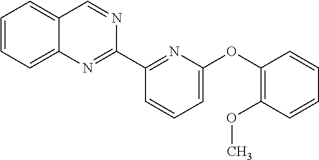 | 1.37 | 330.29 | A | |
| 16 | 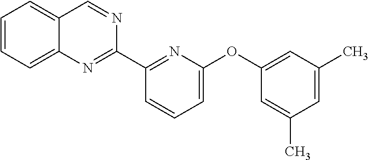 | 1.73 | 328.25 | A | |
| 17 | 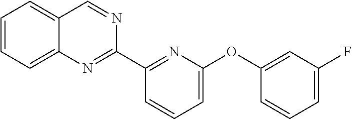 | 1.54 | 318.10 | A | |
| 18 | 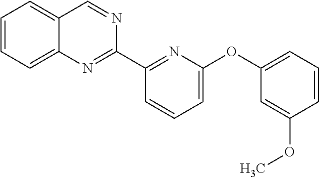 | 1.49 | 330.23 | A | |
| 19 | 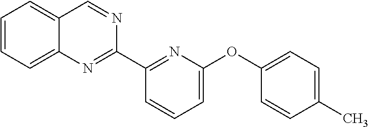 | 1.6 | 314.25 | A | |
| 20 | 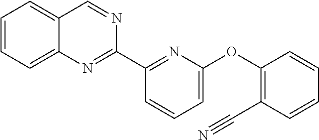 | 1.38 | 325.10 | A | |
| 21 | 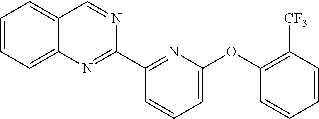 | 1.7 | 368.17 | A | |
| 22 | 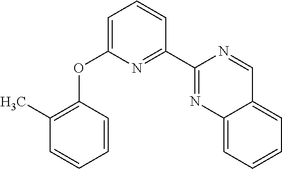 | X | X | X | oil |
| 23 | 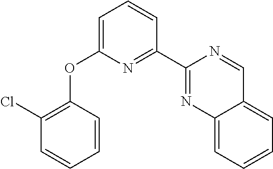 | X | X | X | 137-138 |
| 24 | 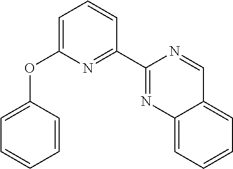 | 1.81 | 300.00 | E | oil |

Table 4 shows retention time and (M+H)⁺ value and/or melting point value measured for selected compounds of the formula I.a where R¹ is H and A is optionally substituted aryloxy

TABLE 5

| | | RT | (M + H)⁺ | | MP |
|---|---|---|---|---|---|
| Entry | Structure | (min) | (measured) | LC/MS | ° C. |
| 1 | 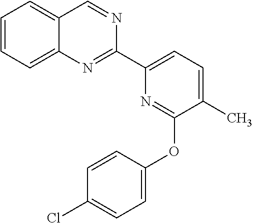 | 2.04 | 348.07 | B | |
| 2 | 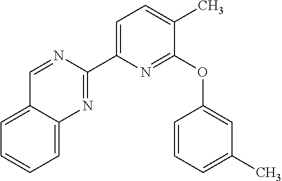 | 1.96 | 328.14 | B | |
| 3 | 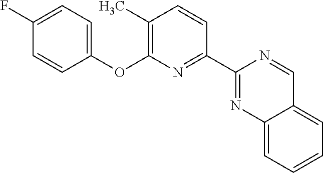 | 1.91 | 332.10 | B | 137-138 |
| 4 | 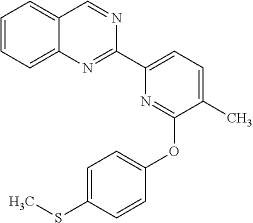 | 2.02 | 360.07 | B | |
| 5 | 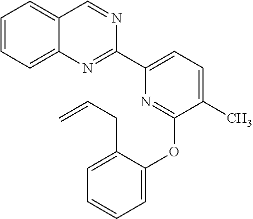 | 2.1 | 354.16 | B | |
| 6 | 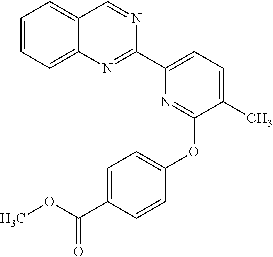 | 1.87 | 372.09 | B | |
| 7 | 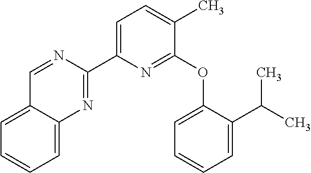 | 2.21 | 356.14 | B | |
| 8 | 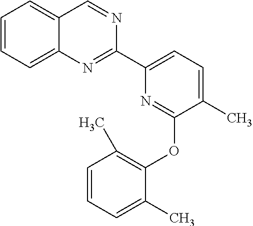 | 2.08 | 342.16 | B | |
| 9 | 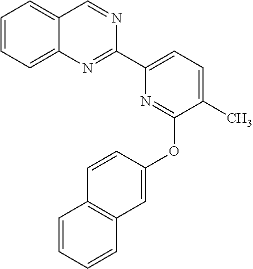 | 2.11 | 364.10 | B | |
| 10 | 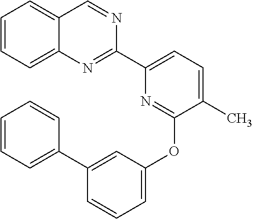 | 2.27 | 390.14 | B | |
| 11 | 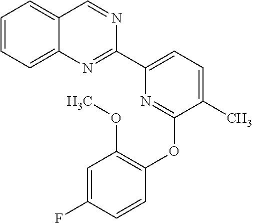 | 1.84 | 362.09 | B | |
| 12 | 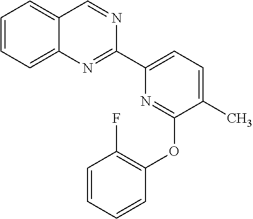 | 1.88 | 332.10 | B | |
| 13 | 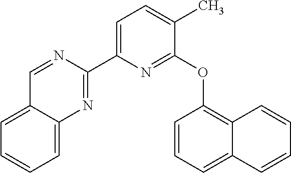 | 2.09 | 364.10 | B | |
| 14 | 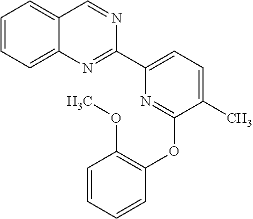 | 1.76 | 344.11 | B | |
| 15 | 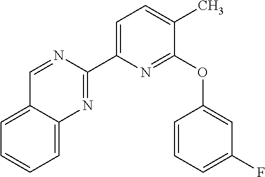 | 1.92 | 332.10 | B | |
| 16 | 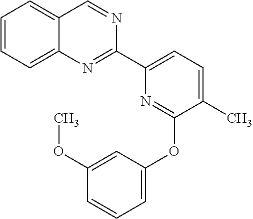 | 1.87 | 344.11 | B | |
| 17 | 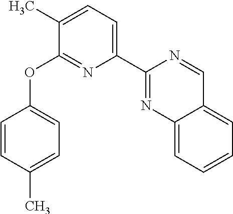 | 1.98 | 328.14 | B | |
| 18 | 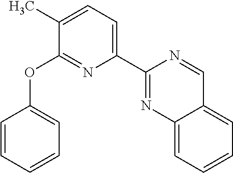 | | | | oil |
| 19 | 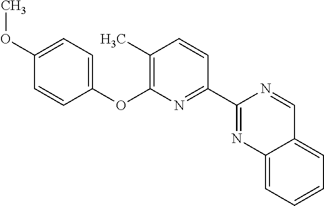 | | | | 125-126 |
| 20 | 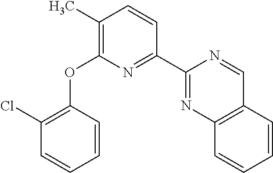 | 2.02 | 349.00 | E | |

Table 5 shows retention time and (M+H)⁺ value and/or melting point value measured for selected compounds of the formula I.a where R1 is Methyl and A is optionally substituted aryloxy

TABLE 6

| | | RT | (M + H)⁺ | | MP |
|---|---|---|---|---|---|
| Entry | Structure | (min) | (measured) | LC/MS | ° C. |
| 1 | 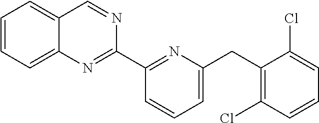 | 1.76 | 366.00 | A | |
| 2 | 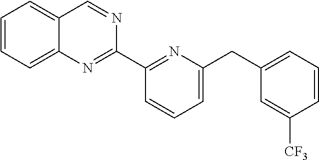 | 1.73 | 366.28 | A | |
| 3 | 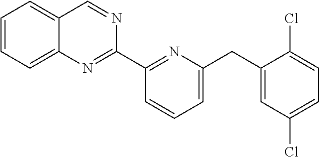 | 1.86 | 366.01 | A | |
| 4 | 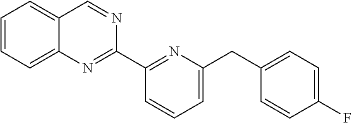 | 1.41 | 316.15 | A | |
| 5 | 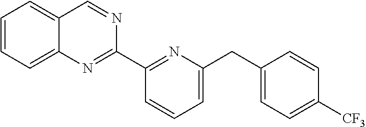 | 1.77 | 366.14 | A | |
| 6 | 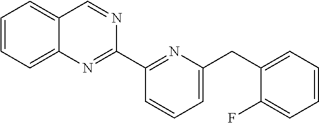 | 1.45 | 316.13 | A | |
| 7 | 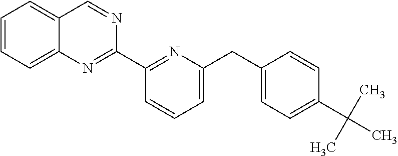 | 1.89 | 354.27 | A | |
| 8 | 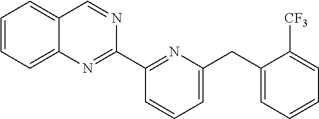 | 1.78 | 366.26 | A | |
| 9 | 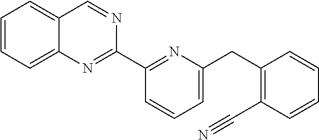 | 1.41 | 323.20 | A | |
| 10 | 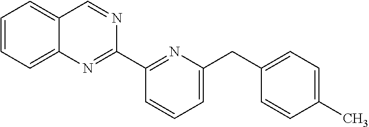 | 1.46 | 312.29 | A | |
| 11 | 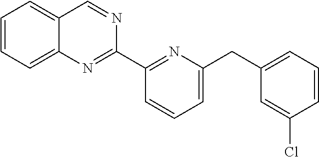 | 1.62 | 332.07 | A | |
| 12 | 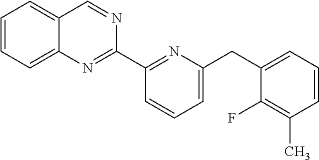 | 1.61 | 330.20 | A | |
| 13 | 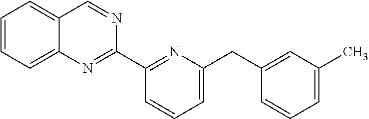 | 1.46 | 312.23 | A | |
| 14 | 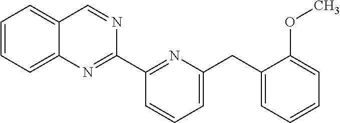 | 1.14 | 328.14 | A | |
| 15 | 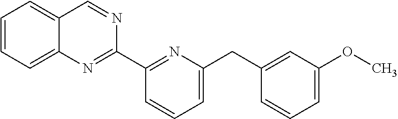 | 1.34 | 328.29 | A | |
| 16 | 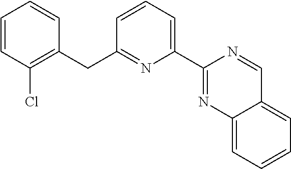 | | | | 132-133 |
| 17 | 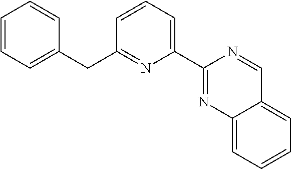 | | | | 102-103 |
| 18 | 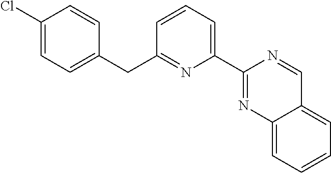 | | | | 112-113 |
| 19 | 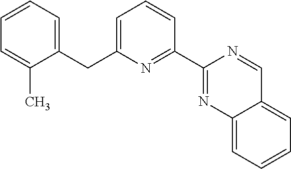 | | | | oil |
| 20 | 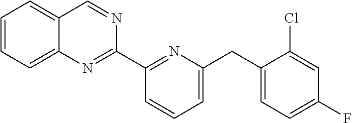 | 2.21 | 350.27 | B | |
| 21 | 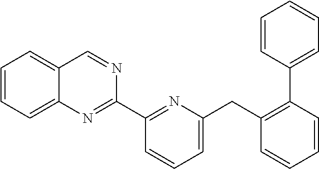 | 2.27 | 374.31 | B | |
| 22 | 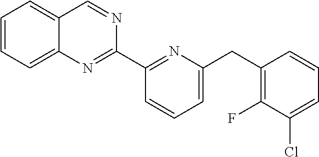 | 2.21 | 350.27 | B | |
| 23 | 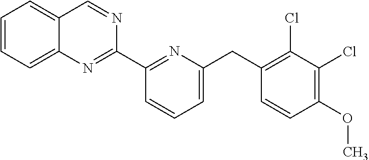 | 2.27 | 396.21 | B | |
| 24 | 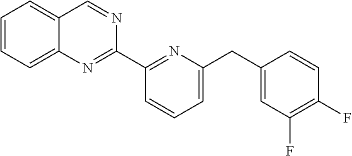 | 2.09 | 334.28 | B | |
| 25 | 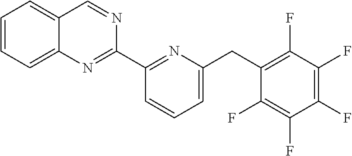 | 2.35 | 388.22 | B | |
| 26 | 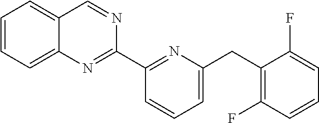 | 2.11 | 334.28 | B | |
| 27 | 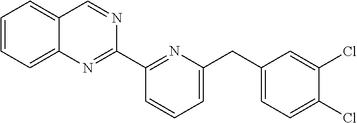 | 2.33 | 366.19 | B | |
| 28 | 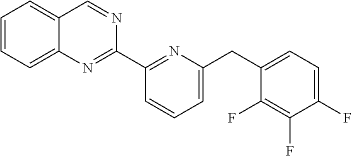 | 2.19 | 352.28 | B | |
| 29 | 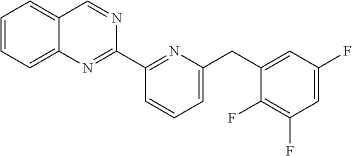 | 2.19 | 352.28 | B | |
| 30 | 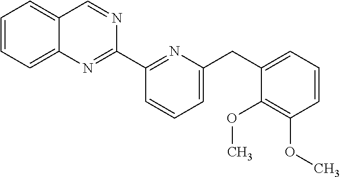 | 1.87 | 358.33 | B | |
| 31 | 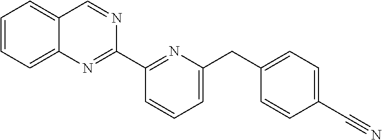 | 1.91 | 323.30 | B | |
| 32 | 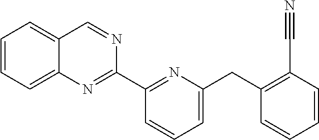 | 1.95 | 323.30 | B | |
| 33 | 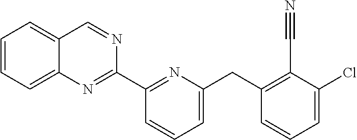 | 2.13 | 357.28 | B | |
| 34 | 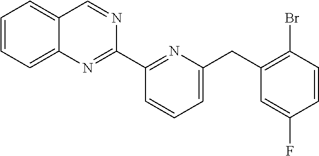 | 2.25 | 394.20 | B | |
| 35 | 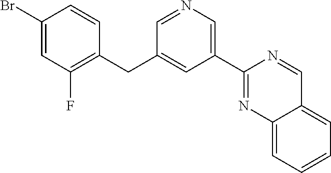 | 2.29 | 394.20 | B | |
| 36 | 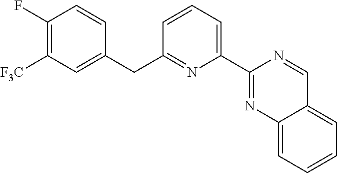 | 2.29 | 384.25 | B | |
| 37 | 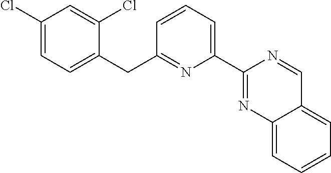 | 2.35 | 366.19 | B | |
| 38 | 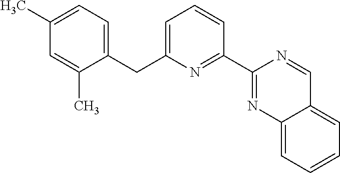 | 2.15 | 326.36 | B | |
| 39 | 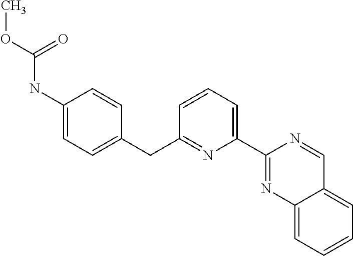 | 1.79 | 371.32 | B | |
| 40 | 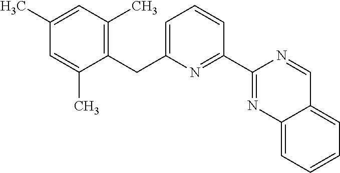 | 2.29 | 340.33 | B | |
| 41 | 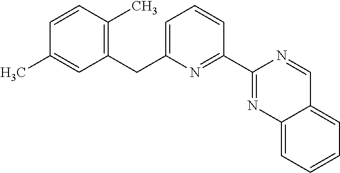 | 2.19 | 326.36 | B | |
| 42 | 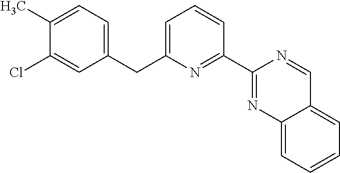 | 2.27 | 346.31 | B | |
| 43 | 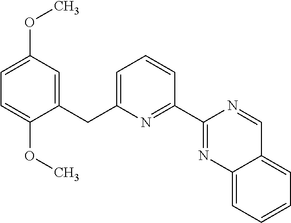 | 1.87 | 358.33 | B | |

Table 6 shows retention time and (M+H)⁺ value and/or melting point value measured for selected compounds of the formula I.a where R¹ is H and A is optionally substituted arylalkyl.

TABLE 7

| | | RT | (M + H)⁺ | | MP |
|---|---|---|---|---|---|
| Entry | Structure | (min) | (measured) | LC/MS | ° C. |
| 1 | 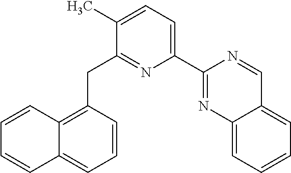 | 1.59 | 362.15 | B | |
| 2 | 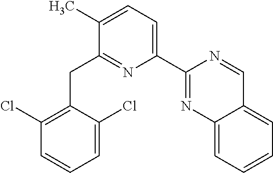 | 1.8 | 380.01 | B | |
| 3 | 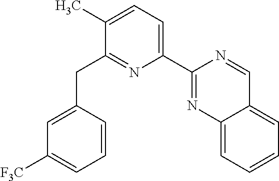 | 1.78 | 380.11 | B | |
| 4 | 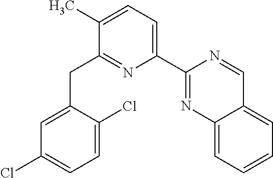 | 1.93 | 380.07 | B | |
| 5 | 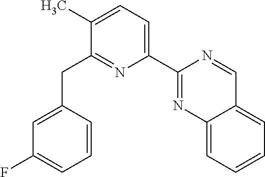 | 1.46 | 330.15 | B | |
| 6 | 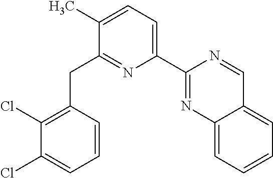 | 1.89 | 380.05 | B | |
| 7 | 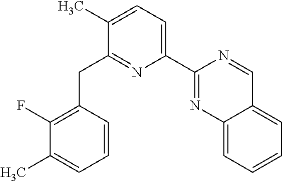 | 1.6 | 344.17 | B | |
| 8 | 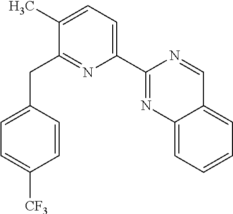 | 1.81 | 380.14 | B | |
| 9 | 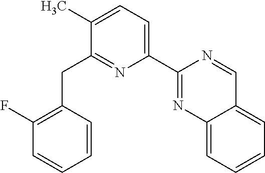 | 1.49 | 330.15 | B | |
| 10 | 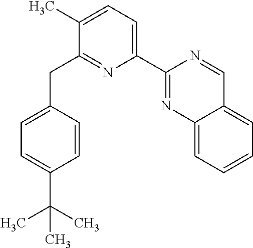 | 1.82 | 368.19 | B | |
| 11 | 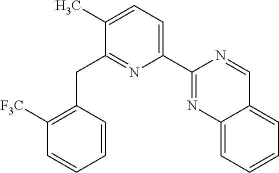 | 1.83 | 380.09 | B | |
| 12 | 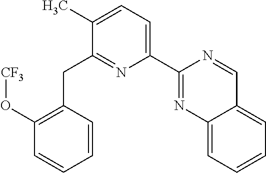 | 1.84 | 396.11 | B | |
| 13 | 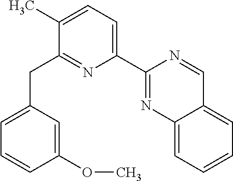 | 1.35 | 342.16 | B | |
| 14 | 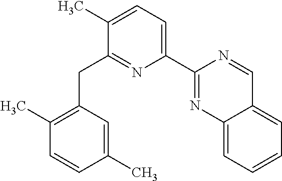 | 1.47 | 340.17 | B | |
| 15 | 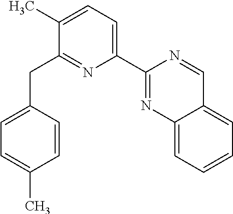 | 1.47 | 326.19 | B | |
| 16 | 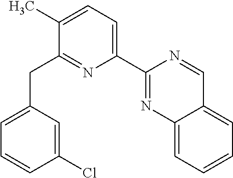 | 1.66 | 346.12 | B | |
| 17 | 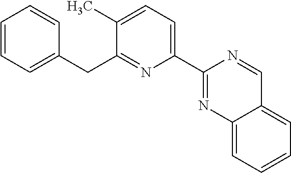 | | | | 164-165 |
| 18 | 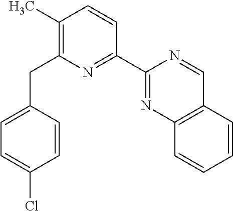 | | | | 171-172 |
| 19 | 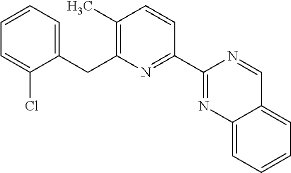 | | | | 166-167 |
| 20 | 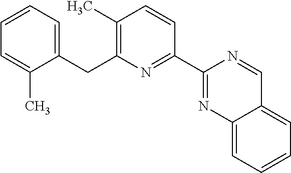 | | | | 177-180 |
| 21 | 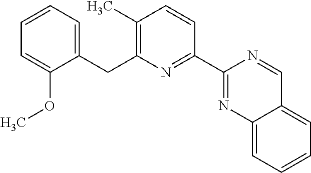 | | | | 140-146 |

Table 7 shows retention time and (M+H)⁺ value and/or melting point value measured for selected compounds of the formula I.a where R¹ is Methyl and A is optionally substituted arylalkyl.

TABLE 8

| | | RT | (M + H)⁺ | | MP |
|---|---|---|---|---|---|
| Entry | Structure | (min) | (measured) | LC/MS | ° C. |
| 1 | 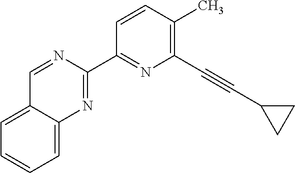 | 1.69 | 286.22 | A | |
| 2 | 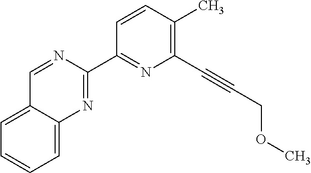 | 1.25 | 290.20 | A | |
| 3 | 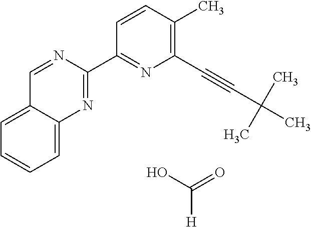 | 1.8 | 302.24 | A | |
| 4 | 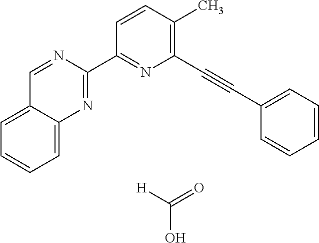 | 1.79 | 322.19 | A | |
| 6 | 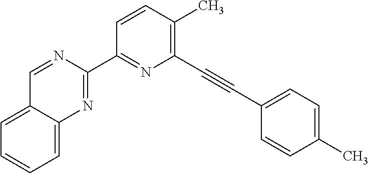 | 1.99 | 336.19 | A | |
| 7 | 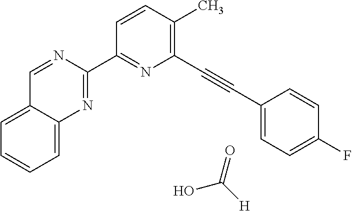 | 1.87 | 340.16 | A | |
| 8 | 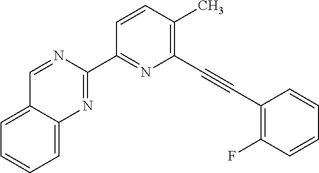 | 1.84 | 340.16 | A | |
| 9 | 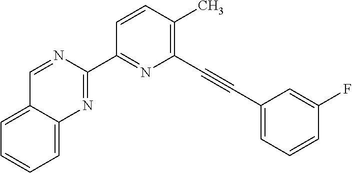 | 1.83 | 340.16 | A | |
| 10 | 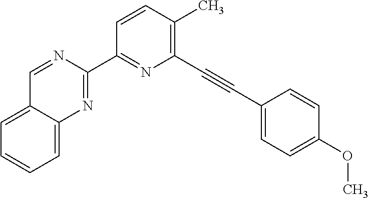 | 1.88 | 352.16 | A | |
| 11 | 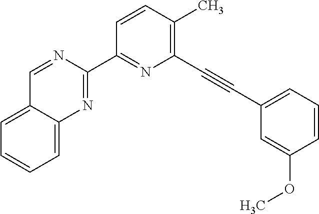 | 1.83 | 352.16 | A | |
| 12 | 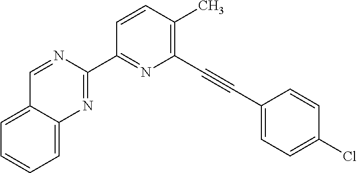 | 2.03 | 356.11 | A | |
| 13 | 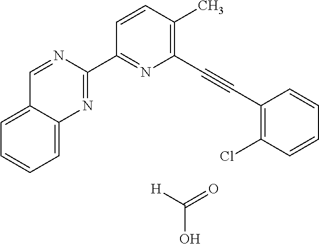 | 1.96 | 356.12 | A | |
| 14 | 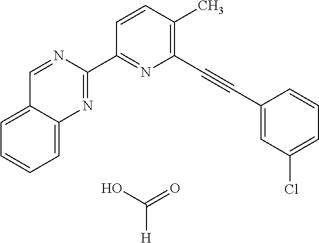 | 1.97 | 356.12 | A | |
| 15 | 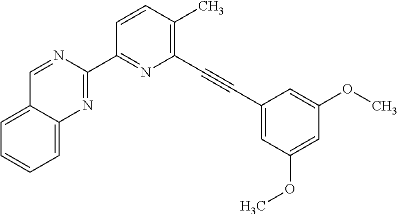 | 1.92 | 382.16 | A | |
| 16 | 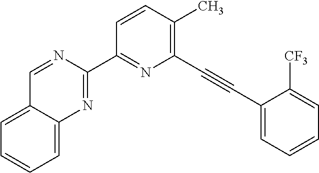 | 1.98 | 390.14 | A | |
| 17 | 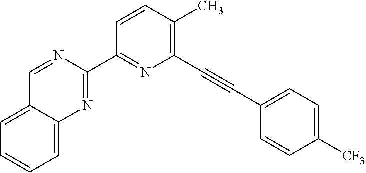 | 2.07 | 390.14 | A | |
| 18 | 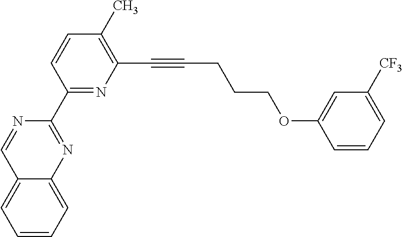 | 2.18 | 448.18 | A | |
| 19 | 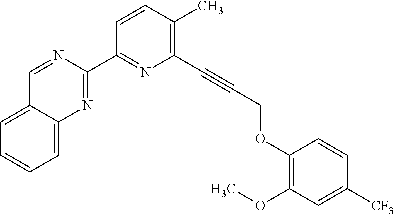 | 1.91 | 450.15 | A | |
| 20 | 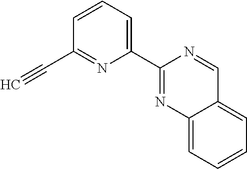 | | | | 193-194 |
| 21 | 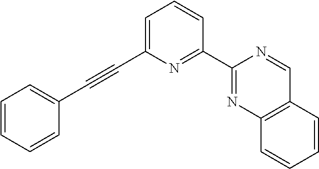 | | | | 136-137 |
| 22 | 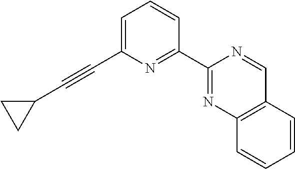 | 1.77 | 272 | E | |
| 23 | 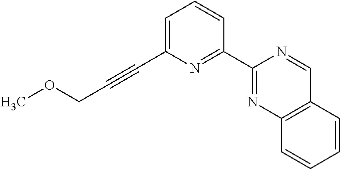 | 1.77 | 272 | E | |

Table 8 shows retention time and (M+H)⁺ value and/or melting point value measured for selected compounds of the formula I.a where R¹ is Methyl or H and A is optionally substituted C₂₋₈-alkynyl.

TABLE 9

| | | RT | (M + H)⁺ | | MP |
|---|---|---|---|---|---|
| Entry | Structure | (min) | (measured) | LC/MS | ° C. |
| 1 | 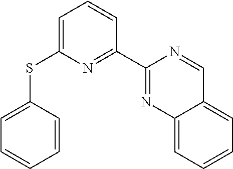 | | | | 161-162 |
| 2 | 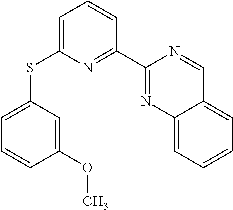 | 1.91 | 346.00 | E | oil |
| 3 | 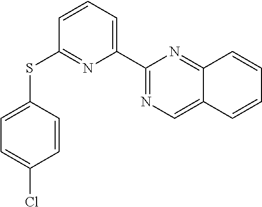 | | | | 151-152 |
| 4 | 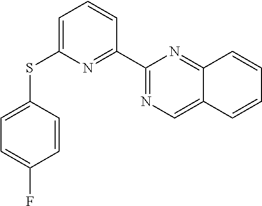 | | | | 166-168 |
| 5 | 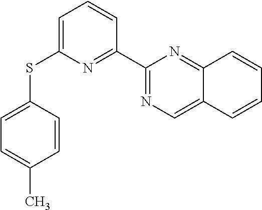 | | | | 156-158 |
| 6 | 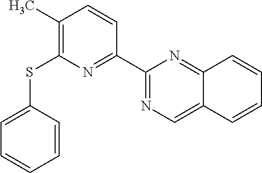 | | | | 124-126 |
| 7 | 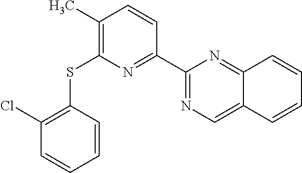 | | | | 142-144 |
| 8 | 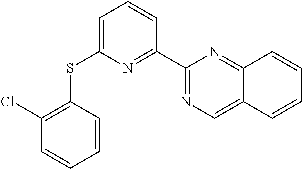 | | | | 135-138 |
| 9 | 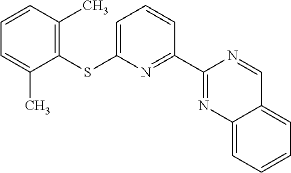 | | | | 126-128 |

Table 9 shows retention time and (M+H)⁺ value and/or melting point value measured for selected compounds of the formula I.a where R¹ is methyl or H and A is arylthio

TABLE 10

| | | RT | (M + H)⁺ | | MP |
|---|---|---|---|---|---|
| Entry | Structure | (min) | (measured) | LS/MS | ° C. |
| 1 | 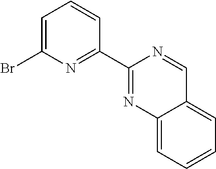 | 1.6 | 288.00 | E | |
| 2 | 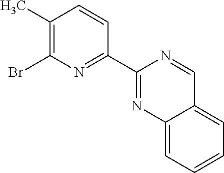 | 1.71 | 302 | E | 168-169 |
| 3 | 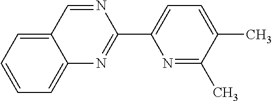 | 0.29 | 236.19 | B | |
| 4 | 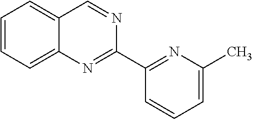 | 0.39 | 222.19 | A | 134-136 |
| 5 | 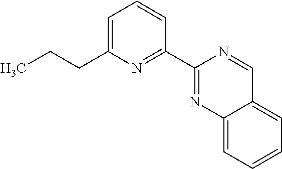 | 1.34 | 250 | E | |
| 6 | 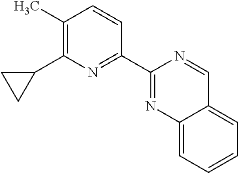 | 1.45 | 262 | E | 158-159 |
| 7 | 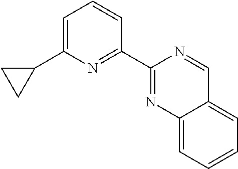 | 1.31 | 248 | E | 139-140 |
| 8 | 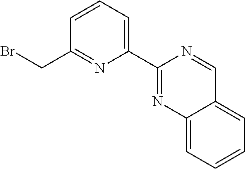 | 1.58 | 302 | E | 142-143 |
| 9 | 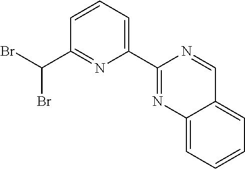 | 1.78 | 380 | E | |
| 10 | 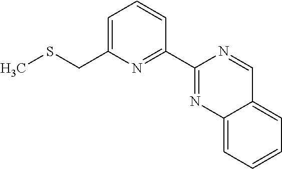 | 1.51 | 268 | E | 101-103 |
| 11 | 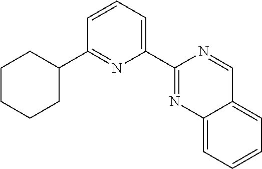 | 1.66 | 290 | E | |
| 12 | 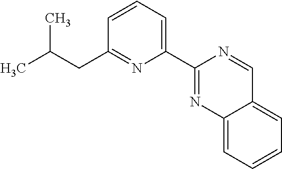 | 1.46 | 264 | E | |
| 13 | 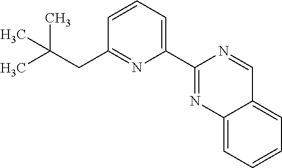 | 1.57 | 278 | E | 106-107 |
| 14 | 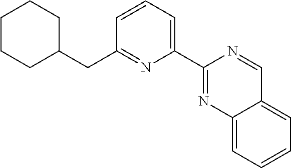 | 1.74 | 304 | E | 95-96 |
| 15 | 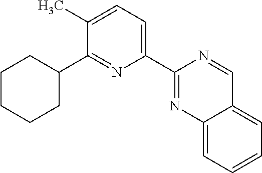 | 1.71 | 304 | E | 173-174 |
| 16 | 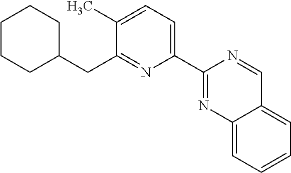 | 1.68 | 318 | E | 180-181 |
| 17 | 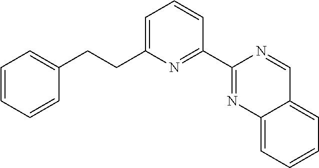 | 1.7 | 312 | E | |
| 18 | 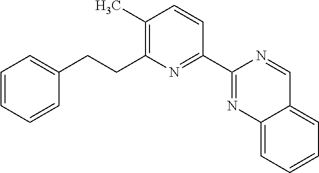 | 1.64 | 326 | E | |
| 19 | 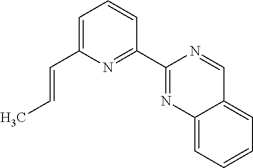 | 1.46 | 248.00 | E | |
| 20 | 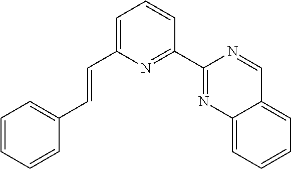 | 1.83 | 310 | E | 128-129 |
| 21 | 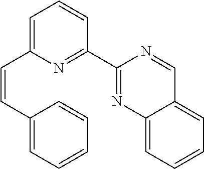 | 1.78 | 310 | E | |
| 22 | 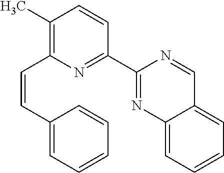 | 1.7 | 324 | E | 129-130 |
| 23 | 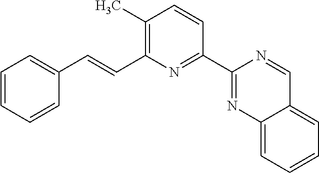 | 1.94 | 324 | E | |
| 24 | 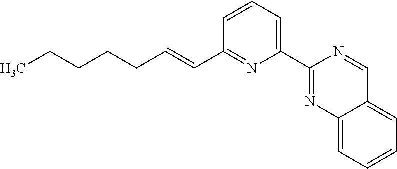 | 1.58 | 304.00 | A | |
| 25 | 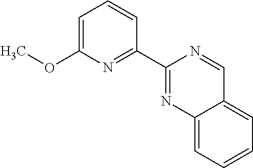 | 1.51 | 238 | E | |
| 26 | 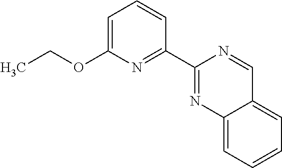 | 1.64 | 252 | E | 110-112 |
| 27 | 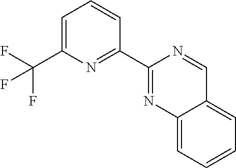 | | | | 136-138 |
| 28 | 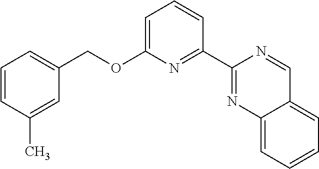 | 2.01 | 328 | E | 68-70 |
| 29 | 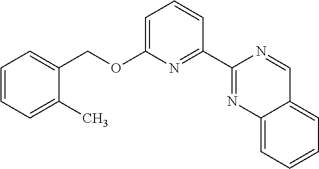 | 1.99 | 328 | E | 72-75 |
| 30 | 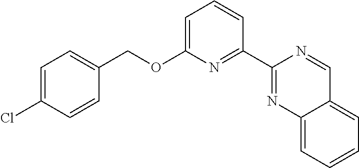 | 2.03 | 348 | E | 111-114 |
| 31 | 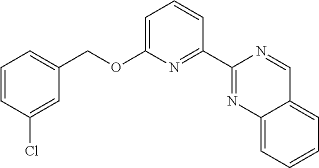 | 2.04 | 348 | E | 111-115 |
| 32 | 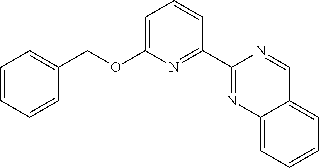 | 2.01 | 314 | E | |
| 33 | 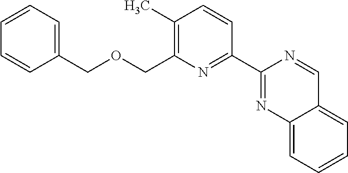 | | | | 103-105 |
| 34 | 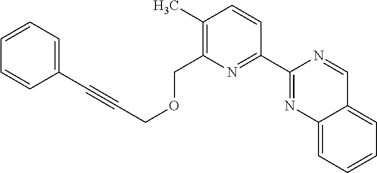 | 1.86 | 366 | E | |
| 35 | 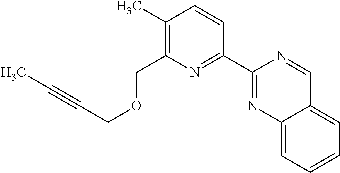 | | | | 85-87 |

Table 10 shows retention time and (M+H)⁺ value and/or melting point value measured for selected compounds of the formula I.a where R¹ is H or methyl and A is halogen, unsubstituted and substituted C₁₋₈ alkyl, C₂₋₈ alkenyl, C₃₋₁₀ cycloalkyl, substituted and unsubstituted C₁₋₈ alkoxy, C₁₋₈ haloalkyl and arylalkyloxy

TABLE 11

| | | RT | (M + H)⁺ | | MP |
|---|---|---|---|---|---|
| Entry | Structure | (min) | (measured) | LC/MS | ° C. |
| 1 | 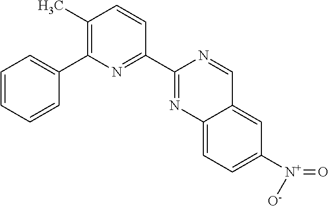 | 1.89 | 343 | E | 201-204 |
| 2 | 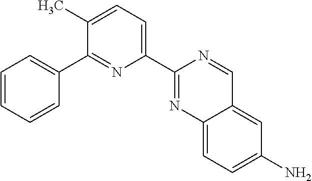 | 1.46 | 313 | E | 209-212 |
| 3 | 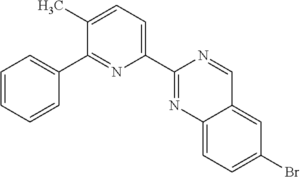 | 2.00 | 378 | E | 188-189 |
| 4 | 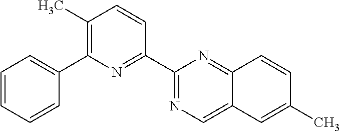 | 1.86 | 312 | E | 164-166 |
| 5 | 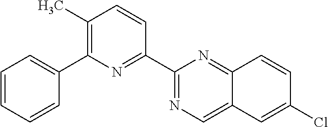 | 1.93 | 332 | E | 184-185 |
| 6 | 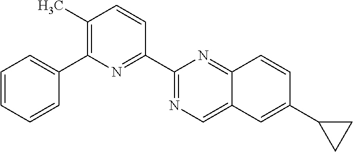 | 1.98 | 338 | E | 155-157 |
| 7 | 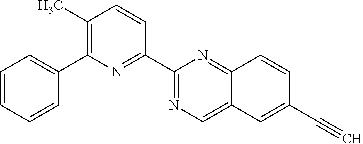 | 1.92 | 322 | E | 183-185 |
| 8 | 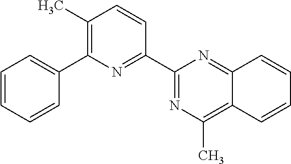 | 1.79 | 312 | E | 141-143 |
| 9 | 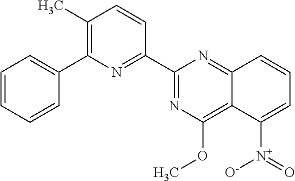 | 1.85 | 373 | E | 245-246 |
| 10 | 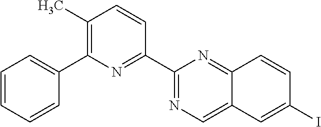 | 2.05 | 424 | E | 195-196 |
| 11 | 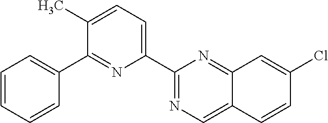 | 1.96 | 332 | E | 172-174 |
| 12 | 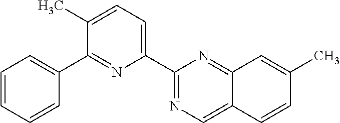 | 1.87 | 312 | E | 156-158 |
| 13 | 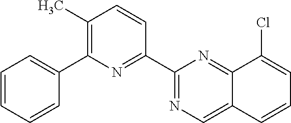 | 1.96 | 332 | E | 179-180 |
| 14 | 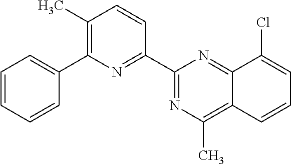 | 1.99 | 346 | E | 175-176 |
| 15 | 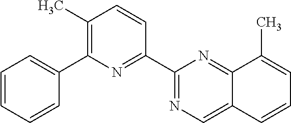 | 1.94 | 312 | E | 157-158 |
| 16 | 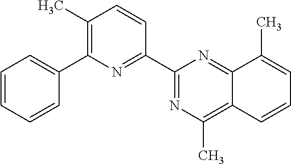 | | | | 154-156 |
| 17 | 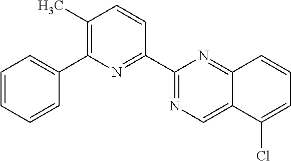 | | | | 219-220 |
| 8 | 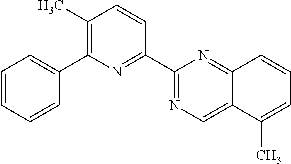 | | | | 187-188 |
| 19 | 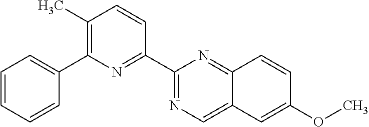 | | | | 169-170 |
| 20 | 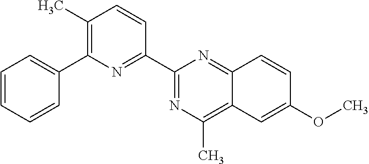 | | | | 164-165 |
| 21 | 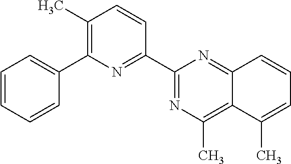 | | | | 178-181 |

Table 11 shows retention time and (M+H)⁺ value and/or melting point value measured for selected compounds of the formula I where R¹ is Methyl, A is unsubstituted phenyl and at least one substituent among R¹, R², R³, R⁴, R⁵, R⁶ is different from H

TABLE 12

| | | RT | (M + H)⁺ | | MP |
|---|---|---|---|---|---|
| Entry | Structure | (min) | (measured) | LC/MS | ° C. |
| 1 | 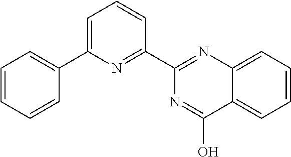 | | | | 181-182 |
| 2 | 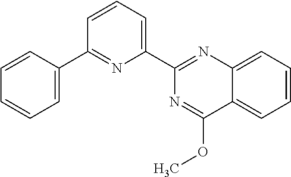 | 1.80 | 328 | E | |
| 3 | 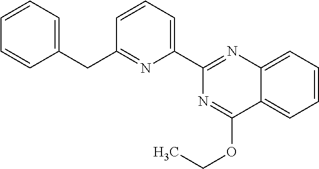 | 1.98 | 348 | E | |
| 4 | 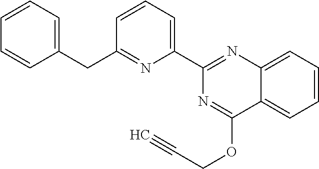 | 1.89 | 352 | E | |
| 5 | 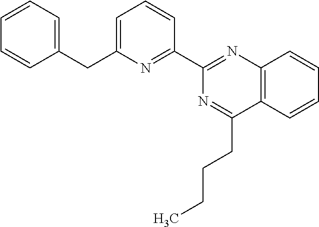 | 2.05 | 354 | E | |
| 6 | 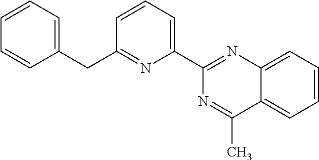 | 1.73 | 312 | E | |
| 7 | 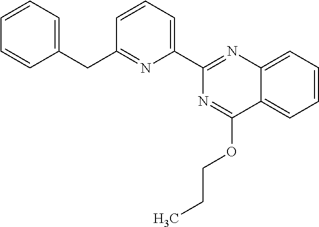 | 2.02 | 356 | E | |
| 8 | 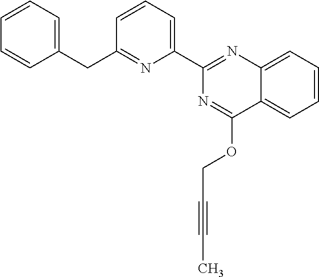 | 1.96 | 366 | E | |
| 9 | 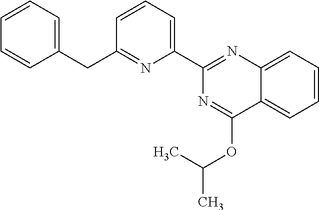 | 1.97 | 356 | E | |
| 10 | 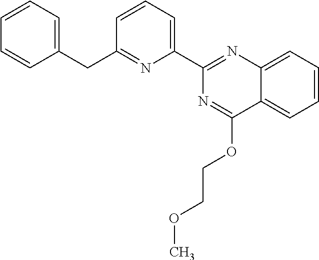 | 1.78 | 372 | E | |
| 11 | 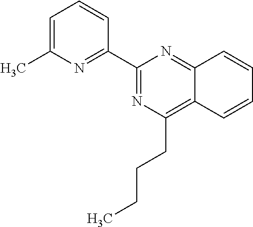 | 1.53 | 278 | E | |
| 12 | 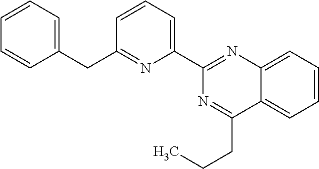 | 1.95 | 340 | E | |
| 13 | 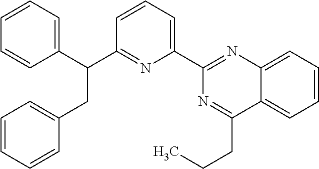 | 2.24 | 430 | E | |
| 14 | 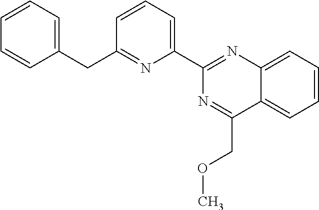 | 1.70 | 342 | E | |
| 15 | 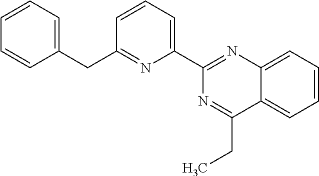 | 1.85 | 326 | E | |
| 16 | 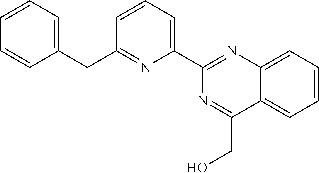 | 1.80 | 328 | E | |

Table 12 shows retention time and (M+H)⁺ value and/or melting point value measured for selected compounds of the formula I where R¹ is H or methyl, A is C₁₋₈ alkyl, or arylalkyl and at least one substituent among R¹, R², R³, R⁴, R⁵, R⁶ is different from H

TABLE 13

| | | RT | (M + H)⁺ | | MP |
|---|---|---|---|---|---|
| Entry | STRUCTURE | (min) | (measured) | LC/MS | ° C. |
| 1 | 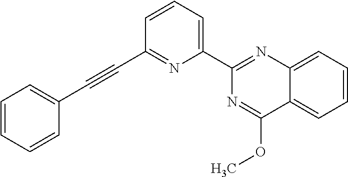 | 2.03 | 338 | E | |
| 2 | 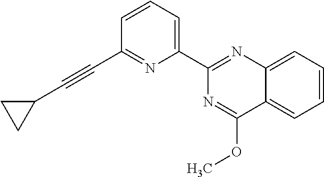 | 1.88 | 302 | E | |
| 3 | 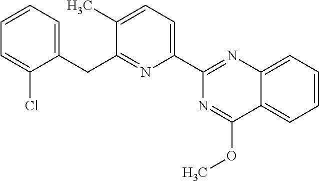 | | | | 167-170 |
| 4 | 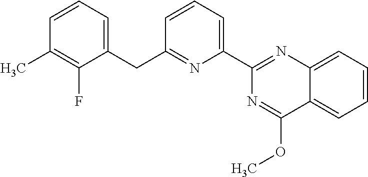 | 1.94 | 360 | E | |
| 5 | 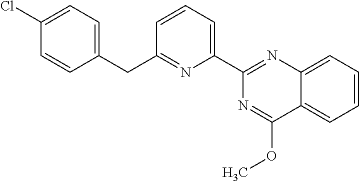 | | | | 121-123 |
| 6 | 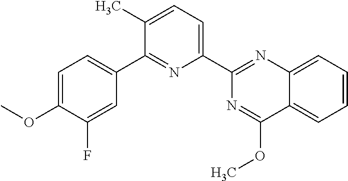 | | | | 173-175 |
| 7 | 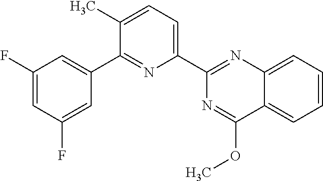 | | | | 165-167 |
| 8 | 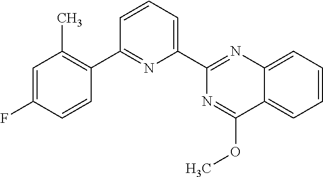 | 1.99 | 346 | E | |
| 9 | 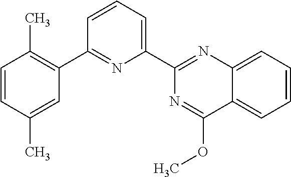 | 2.03 | 342 | E | |
| 10 | 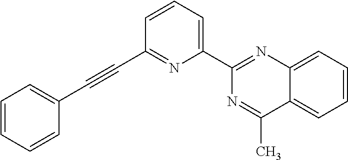 | | | | 131-133 |
| 11 | 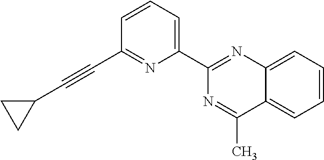 | 1.84 | 286 | E | |
| 12 | 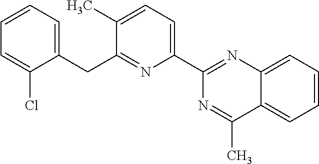 | | | | 137-139 |
| 13 | 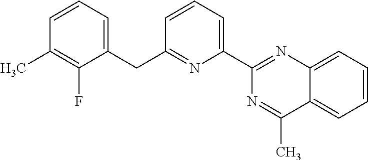 | 1.89 | 344 | E | |
| 14 | 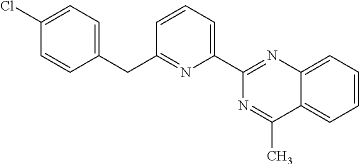 | | | | 114-116 |
| 15 | 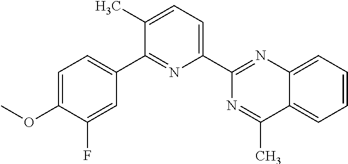 | | | | 186-188 |
| 16 | 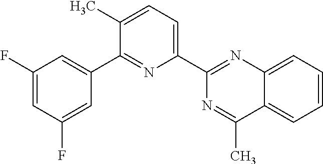 | | | | 172-174 |
| 17 | 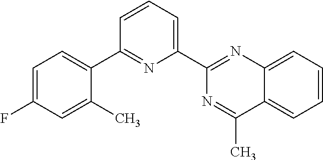 | | | | 187-189 |
| 18 | 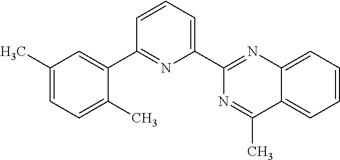 | | | | 99-101 |

Table 13 shows retention time and (M+H)⁺ value and/or melting point value measured for selected compounds of the formula I where R¹ is H or methyl, A is C₂₋₁₀ alkynyl, aryl or arylalkyl and R² is C₁₋₈ alkyl or C₁₋₈ alkoxy.

TABLE 14

| | | RT | (M + H)⁺ | | |
|---|---|---|---|---|---|
| Entry | STRUCTURE | (min) | (measured) | LC/MS | ° C. |
| 1 | 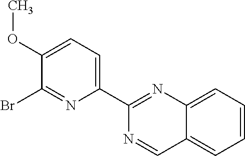 | | | | 189-191 |
| 2 | 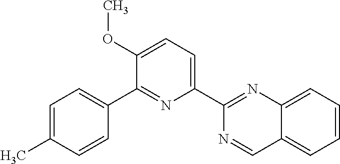 | | | | 131-133 |
| 3 | 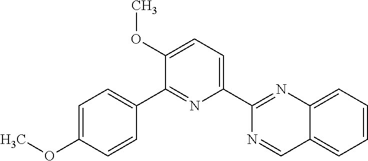 | | | | 155-157 |
| 4 | 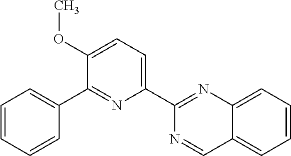 | | | | 168-169 |
| 5 | 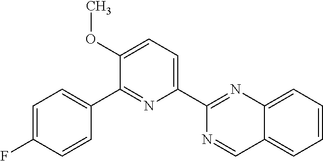 | | | | 153-155 |
| 6 | 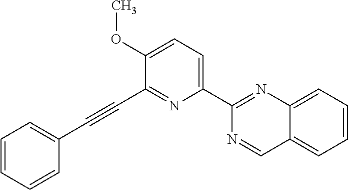 | | | | 58-60 |
| 7 | 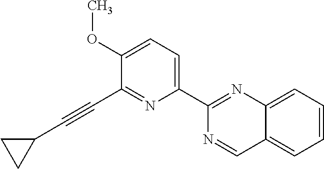 | 1.70 | 302 | E | |
| 8 | 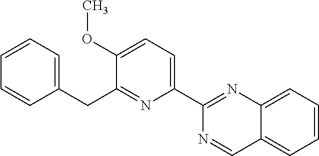 | 1.78 | 328 | E | |
| 9 | 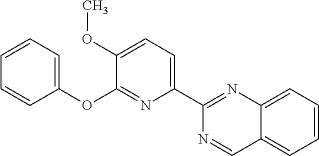 | 1.76 | 330 | E | |

Table 14 shows retention time and (M+H)⁺ value and/or melting point value measured for selected compounds of the formula I where R¹ Methoxy and A is halogen, C₂₋₁₀ alkynyl, aryl, aryloxy and arylalkyl

### Example 6

### Biological Examples

### Alternaria solani/Tomato/Preventative (Alternaria on Tomato)

4-week old tomato plants cv. Roter Gnom are treated with the formulated test compound in a spray chamber. The test plants are inoculated by spraying them with a spore suspension two days after application. The inoculated test plants are incubated at 22/18° C. (day/night) and 95% rh in a greenhouse and the percentage leaf area covered by disease is assessed when an appropriate level of disease appears on untreated check plants (5-7 days after application).

### Compounds (Table/Entry)

3/70, 3/71, 3/72, 3/74, 3/75, 3/76, 3/77, 3/82, 3/83, 3/84, 3/85, 3/86, 3/89, 3/90, 3/92, 3/93, 3/94, 3/95, 3/101, 5/18, 5/4, 6/16, 6/17, 6/18, 7/17, 7/18, 11/4, 4/22, 6/19, 9/2, 2/3, 2/4, 2/6, 2/9, 2/13, 2/15, 2/28, 2/30, 2/32, 2/33, 2/37, 2/38, 2/46, 2/54, 2/55, 2/60, 2/66, 2/68, 2/70, 2/73, 2/90, 2/94, 9/4, 9/6, 9/7, 9/8, 4/10, 6/11, 6/12, 3/9, 3/11, 3/17, 3/21, 3/26, 3/36, 3/37, 3/38, 3/46, 3/53, 3/56, 11/8, 5/13, 7/7, 8/1, 12/6, at 200 ppm give at least 80% disease control in this test when compared to untreated control leaf disks under the same conditions, which show extensive disease development.

Botryotinia fuckeliana (Botrytis cinerea)/Tomato/Preventative (Botrytis on Tomato)

4-week old tomato plants cv. Roter Gnom are treated with the formulated test compound in a spray chamber. The test plants are inoculated by spraying them with a spore suspension two days after application. The inoculated test plants are incubated at 20° C. and 95% rh in a greenhouse and the percentage leaf area covered by disease is assessed when an appropriate level of disease appears on untreated check plants (5-6 days after application).

Compounds (Table/Entry) 3/69, 3/71, 3/72, 3/75, 3/76, 3/83, 3/85, 3/89, 3/90, 3/92, 3/94, 3/101, 5/18, 6/16, 6/17, 7/19, 2/1, 2/6, 2/13, 2/37, 2/55, 2/60, 6/11, 6/12, 10/7, 3/9, 3/21, 3/26, 3/36, 3/38, 3/53, 8/1, at 200 ppm give at least 80% disease control in this test when compared to untreated control leaf disks under the same conditions, which show extensive disease development.

Erysiphe necator (Uncinula necator)/Grape/Preventative (Powdery Mildew on Grape)

5-week old grape seedlings cv. Gutedel are treated with the formulated test compound in a spray chamber. The test plants are inoculated by shaking plants infected with grape powdery mildew above them 1 day after application. The inoculated test plants are incubated at 24/22° C. (day/night) and 70% rh under a light regime of 14/10 h (light/dark) and the percentage leaf area covered by disease is assessed when an appropriate level of disease appears on untreated check plants (7-9 days after application).

Compounds (Table/Entry) 3/75, 3/85, 3/89, 3/90, 3/92, 6/16, 6/17, 2/54, 2/55, 2/68, 10/4, 6/11, 6/12, 10/7, 3/21, 3/38, 3/53, 11/8, 7/7, 12/2, 12/3, 12/6

at 200 ppm give at least 80% disease control in this test when compared to untreated control leaf disks under the same conditions, which show extensive disease development.

Mycosphaerella arachidis (Cercospora arachidicola)/Peanut/Preventative

3-week old peanut plants cv. Georgia Green are treated with the formulated test compound in a spray chamber. The test plants are inoculated by spraying them with a spore suspension on their lower leaf surface one day after application. After an incubation period of 4 days under a plastic hood at 23° C. and 100% rh, the test plants are kept at 23° C./20° C. (day/night) and 70% rh in a greenhouse. The percentage leaf area covered by disease is assessed when an appropriate level of disease appears on untreated check plants (12-14 days after application).

Compounds (Table/Entry) 3/75, 3/76, 3/85, 3/89, 3/90, 3/92, 5/17, 5/3, 6/17, 7/17, 2/1, 2/6, 2/13, 2/26, 2/37, 2/54, 2/55, 6/11, 6/12, 10/7, 10/4, 3/9, 3/26, 3/38, 3/46, 3/53, 3/56, 11/8, 8/1, 12/2, at 200 ppm give at least 80% disease control in this test when compared to untreated control leaf disks under the same conditions, which show extensive disease development.

Mycosphaerella graminicola (Septoria tritici)/Wheat/Preventative (Septoria tritici Leaf Spot on Wheat)

2-week old wheat plants cv. Riband are treated with the formulated test compound in a spray chamber. The test plants are inoculated by spraying a spore suspension on them one day after application. After an incubation period of 1 day at 22° C./21° C. (day/night) and 95% rh, the test plants are kept at 22° C./21° C. (day/night) and 70% rh in a greenhouse. The percentage leaf area covered by disease is assessed when an appropriate level of disease appears on untreated check plants (16-19 days after application).

Compounds (Table/Entry) 3/71, 3/74, 3/75, 3/76, 3/77, 3/82, 3/83, 3/85, 3/89, 3/90, 3/92, 3/93, 3/94, 3/101, 6/16, 6/18, 7/17, 6/19, 2/73, 6/10, 6/11, 6/12, 6/15, 3/9, 11/8, 12/2 at 200 ppm give at least 80% disease control in this test when compared to untreated control leaf disks under the same conditions, which show extensive disease development.

Phytophthora infestans/Potato/Preventative (Late Blight on Potato)

2-week old potato plants cv. Bintje are treated with the formulated test compound in a spray chamber. The test plants are inoculated by spraying them with a sporangia suspension 2 days after application. The inoculated test plants are incubated at 18° C. with 14 h light/day and 100% rh in a growth chamber and the percentage leaf area covered by disease is assessed when an appropriate level of disease appears on untreated check plants (5-7 days after application).

Compounds (Table/Entry) 3/71, 3/72, 3/75, 3/76, 3/77, 3/85, 3/90, 3/92, 5/18, 6/17, 7/17, 2/55, 2/60 at 200 ppm give at least 80% disease control in this test when compared to untreated control leaf disks under the same conditions, which show extensive disease development.

Plasmopara viticola/Grape/Preventative (Grape Downy Mildew)

5-week old grape seedlings cv. Gutedel are treated with the formulated test compound in a spray chamber. The test plants are inoculated by spraying a sporangia suspension on their lower leaf surface one day after application. The inoculated test plants are incubated at 22° C. and 100% rh in a greenhouse and the percentage leaf area covered by disease is assessed when an appropriate level of disease appears on untreated check plants (6-8 days after application).

Compounds (Table/Entry) 3/69, 3/71, 3/72, 3/73, 3/74, 3/75, 3/76, 3/77, 10/3, 3/82, 3/83, 3/84, 3/85, 3/86, 3/89, 3/90, 3/92, 3/93, 3/94, 3/95, 3/101, 5/18, 5/17, 11/2, 5/3, 5/4, 6/16, 6/17, 11/5, 4/22, 7/19, 6/19, 9/2, 2/3, 2/6, 2/9, 2/13, 2/26, 2/28, 2/30, 2/37, 2/46, 2/54, 2/55, 2/60, 2/68, 2/70, 2/73, 2/79, 2/90, 2/94, 4/10, 6/15, 10/7, 3/9, 3/11, 3/21, 3/26, 3/36, 3/37, 3/38, 3/46, 3/53, 3/56, 11/8, 7/7, 8/1, 12/2, at 200 ppm give at least 80% disease control in this test when compared to untreated control leaf disks under the same conditions, which show extensive disease development.

Pyrenophora teres (Helminthosporium teres)/Barley/Preventative (Net Blotch on Barley)

1-week old barley plants cv. Regina are treated with the formulated test compound in a spray chamber. The test plants are inoculated by spraying them with a spore suspension 2 days after application. The inoculated test plants are incubated at 20° C. and 95% rh and the percentage leaf area covered by disease is assessed when an appropriate level of disease appears on untreated check plants (5-7 days after application).

Compounds (Table/Entry) 3/69, 3/70, 3/71, 3/72, 3/73, 3/74, 3/76, 3/82, 3/83, 3/84, 3/85, 3/86, 3/89, 3/90, 3/92, 3/93, 3/94, 3/95, 3/101, 5/18, 5/3, 6/16, 6/17, 6/18, 7/18, 11/4, 11/5, 4/22, 6/19, 9/2, 2/3, 2/6, 2/9, 2/28, 2/32, 2/54, 2/55, 2/90, 9/4, 9/6, 9/7, 9/8, 4/15, 6/11, 6/12, 6/15, 4/23, 3/9, 3/26, 3/38, 3/53, 11/8, 5/13, 7/7, at 200 ppm give at least 80% disease control in this test when compared to untreated control leaf disks under the same conditions, which show extensive disease development.

Phaeosphaeria nodorum (Septoria nodorum)/Wheat/Leaf Disc Preventative (Glume Blotch)

Wheat leaf segments cv. Kanzler are placed on agar in a multiwell plate (24-well format) and sprayed with the formulated test compound diluted in water. The leaf disks are inoculated with a spore suspension of the fungus 2 days after application. The inoculated test leaf disks are incubated at 20° C. and 75% rh under a light regime of 12 h light/12 h darkness in a climate cabinet and the activity of a compound is assessed as percent disease control compared to untreated when an appropriate level of disease damage appears in untreated check leaf disks (5-7 days after application).

Compounds (Table/Entry) 2/38, 2/50, 2/57, 2/60, 2/61, 2/66, 2/104, 2/105, 2/108, 2/110, 2/111, 2/112, 2/113, 2/115, 2/116, 2/117, 2/119, 2/121, 2/124, 2/125, 2/126, 2/128, 2/130, 2/131, 2/132, 2/133, 3/102, 3/103, 3/104, 3/105, 3/106, 3/107, 3/108, 3/109, 3/110, 3/114, 3/118, 3/120, 3/123, 3/124, 3/125, 3/126, 3/128, 3/129, 3/131, 3/132, 3/133, 3/134, 3/135, 3/137, 3/138, 3/139, 3/142, 4/24, 6/20, 6/21, 6/22, 6/23, 6/24, 6/26, 6/27, 6/28, 6/29, 6/30, 6/31, 6/32, 6/33, 6/34, 6/35, 6/37, 6/38, 6/39, 6/40, 6/41, 6/42, 6/43, 7/20, 7/21, 10/17, 10/20, 10/21, 10/22, 10/23, 11/11, 11/12, 11/13, 11/14, 11/15, 11/16, 11/18, 11/19, 11/20, 11/21, 12/2, 12/3, 12/5, 12/6, 12/9, 12/10, 12/13, 12/15, 12/16, 14/2, 14/3, 14/4, 14/5 at 200 ppm give at least 80% disease control in this test when compared to untreated control leaf disks under the same conditions, which show extensive disease development.

Although the invention has been described with reference to preferred embodiments and examples thereof, the scope of the present invention is not limited only to those described embodiments. As will be apparent to persons skilled in the art, modifications and adaptations to the above-described invention can be made without departing from the spirit and scope of the invention, which is defined and circumscribed by the appended claims. All publications cited herein are hereby incorporated by reference in their entirety for all purposes to the same extent as if each individual publication were specifically and individually indicated to be so incorporated by reference.

1. A compound of formula I: 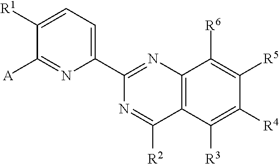
wherein:
R¹ is hydrogen, hydroxyl, halo, cyano, C₁₋₈ alkyl, C₁₋₈ haloalkyl, C₁₋₈ alkoxy, C₁₋₈ haloalkoxy, C₁₋₈ alkylthio or C₃₋₁₀ cycloalkyl;
R² is hydrogen, hydroxyl, halo, C₁₋₈ alkyl or C₁₋₈ alkoxy;
R³, R⁴, R⁵ and R⁶ are, independently, hydrogen, hydroxyl, halo, cyano, nitro, amino, C₁₋₈ alkyl, C₂₋₈ alkenyl, C₂₋₈ alkynyl, C₁₋₈ haloalkyl, C₁₋₈ alkoxy, C₁₋₈ haloalkoxy, C₁₋₈ alkylthio or C₃₋₁₀ cycloalkyl;
A is halo, C₁₋₈ alkyl, C₂₋₈ alkenyl, C₂₋₈ alkynyl, C₁₋₈ haloalkyl, C₁₋₈ alkoxy, C₃₋₁₀ cycloalkyl, C₃₋₁₀ cycloalkyloxy, aryl, arylalkyl, aryloxy, arylalkyloxy or arylthio;
or a salt or a N-oxide thereof.

2. A compound of claim 1, wherein R¹ is hydrogen, halo, C₁₋₈ alkyl, C₁₋₈ alkoxy, C₁₋₈ haloalkyl, or C₁₋₈ alkylthio.

3. A compound of claim 2, wherein R¹ is hydrogen, halo, C₁₋₃ alkyl, C₁₋₃ alkoxy, C₁₋₃ haloalkyl, or C₁₋₃ alkylthio.

4. A compound of claim 3, wherein R¹ is hydrogen, C₁₋₃ alkyl, C₁₋₃ alkoxy or C₁₋₄ haloalkyl.

5. A compound of claim 4, wherein R¹ is hydrogen, methyl, ethyl, methoxy or trifluoromethyl.

6. A compound of claim 5, wherein R¹ is hydrogen or methyl.

7. A compound of claim 1, wherein R² is hydrogen, hydroxyl, halo, C₁₋₃ alkyl or C₁₋₃ alkoxy.

8. A compound of claim 7, wherein R² is hydrogen, hydroxyl, fluoro, chloro, methyl or methoxy.

9. A compound of claim 8, wherein R² is hydrogen, hydroxyl, chloro or methoxy.

10. A compound of claim 1, wherein R³, R⁴, R⁵ and R⁶ are, independently, hydrogen, halo, cyano, C₁₋₈ alkyl, C₁₋₈ haloalkyl, C₁₋₈ alkoxy or C₁₋₈ haloalkoxy.

11. A compound of claim 10, wherein R³, R⁴, R⁵ and R⁶ are, independently, hydrogen, halo, cyano, C₁₋₃ alkyl, C₁₋₃ haloalkyl, C₁₋₃ alkoxy or C₁₋₃ haloalkoxy.

12. A compound of claim 11, wherein R³, R⁴, R⁵ and R⁶ are, independently, hydrogen, halo, cyano, C₁₋₃ alkyl or C₁₋₃ alkoxy.

13. A compound of claim 12, wherein R³, R⁴, R⁵ and R⁶ are, independently, hydrogen, bromo, chloro, fluoro, methyl or methoxy.

14. A compound of claim 1, wherein A is halo, C₁₋₈ haloalkyl, optionally substituted aryl, optionally substituted arylalkyl or optionally substituted aryloxy.

15. A compound of claim 14, wherein A is halo, optionally substituted phenyl, optionally substituted naphthyl, optionally substituted benzyl, optionally substituted phenoxy, optionally substituted phenylthio or optionally substituted arylethynyl.

16. A compound of claim 15, wherein A is halogen, optionally substituted phenyl, optionally substituted benzyl or optionally substituted phenoxy.

17. A compound of claim 16, wherein A is optionally substituted phenyl.

18. A compound of claim 1, where in R¹ is hydrogen, C₁₋₃ alkyl, C₁₋₃ haloalkyl or C₁₋₃ alkoxy, R² is hydrogen, hydroxyl, halo, C₁₋₃ alkyl or C₁₋₃ alkoxy, R³, R⁴, R⁵ and R⁶ are, independently hydrogen, halo, C₁₋₃ alkyl, C₁₋₃ haloalkyl or C₁₋₃ alkoxy and A is halo, optionally substituted aryl, optionally substituted arylalkyl, optionally substituted aryloxy or optionally substituted arylthio, wherein the optional substituents are selected from halo, C₁₋₃ alkyl, C₁₋₃ haloalkyl and C₁₋₃ alkoxy or a combination of any of these substituents.

19. A compound of claim 18, wherein R¹ is hydrogen, methyl, ethyl, trifluoromethyl or methoxy, R² is hydrogen, hydroxyl, fluoro, chloro, methyl or methoxy, R³, R⁴, R⁵ and R⁶ are, independently, hydrogen, fluoro, chloro, methyl, trifluoromethyl or methoxy and A is bromo, iodo, optionally substituted phenyl, optionally substituted phenylmethyl, optionally substituted phenoxy, optionally substituted phenylthio or optionally substituted phenylethynyl, wherein the optional substituents are selected from fluoro, chloro, methyl, trifluoromethyl or methoxy or a combination of any of these substituents.

20. A compound of claim 1, which is:
2-(5-methyl-6-o-tolylpyridin-2-yl)-quinazoline (Compound I.a 096);
2-[6-(4-fluoro-3-methylphenyl)-5-methylpyridin-2-yl]-quinazoline (Compound I.a 681),
2-[6-(3-fluoro-4-methoxy-phenyl)-5-methylpyridin-2-yl]-quinazoline (Compound I.a 581);
2-[6-(3,5-dimethylphenyl)-5-methylpyridin-2-yl]-quinazoline (Compound I.a 881);
2-[6-(3,5-difluorophenyl)-5-methylpyridin-2-yl]-quinazoline (Compound I.a 831);
2-[6-(3,4-difluorophenyl)-5-methylpyridin-2-yl]-quinazoline (Compound I.a 421);
6-Methyl-2-(5-methyl-6-phenylpyridin-2-yl)-quinazoline (Compound I.s 021);
2-(6-benzylpyridin-2-yl)-quinazoline (Compound I.a 017);
2-[6-(2-chlorobenzyl)-pyridin-2-yl]-quinazoline (Compound I.a 067);
2-[6-(2-methylbenzyl)-pyridin-2-yl]-quinazoline (Compound I.a 092);
2-(6-benzyl-5-methylpyridin-2-yl)-quinazoline (Compound I.a 022); and
2-(6-benzylpyridin-2-yl)-6-methylquinazoline (Compound I.s 017).

21. A process for the preparation of a compound of formula I, wherein R² is hydrogen, which comprises:
(i) reacting a compound of formula II with an oxidation agent: 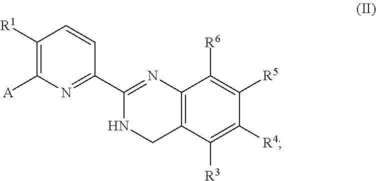
or
(ii) reacting a compound of formula (VIII) with an oxidation agent: 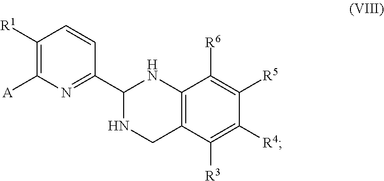
or
(iii) reacting a compound of formula XIII or a salt thereof: 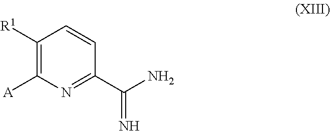
and a benzaldehyde of formula XIV: 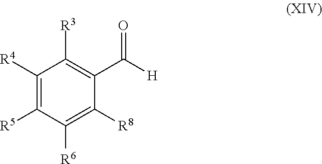
with a base,
wherein R¹, R³, R⁴, R⁵, R⁶ and A are as defined in claim 1 and R⁸ is a halogen or an amino group.

22. A method of preventing and/or controlling fungal infection in plants and/or plant propagation material comprising applying to the plant or plant propagation material or the locus thereof a fungicidally effective amount of a compound of formula I.

23. A composition for the control of fungal infection comprising a compound of formula I and an agriculturally acceptable carrier or diluent.

24. A composition of claim 23, which further comprises at least one additional fungicidally active compound.

25. A composition of claim 24, wherein the additional fungicidally active compound is acibenzolar-S-methyl, azoxystrobin, chlorothalonil, cyproconazole, cyprodinil, difenoconazole, fenpropidin, fluazinam, fludioxonil, hexaconazole, isopyrazam, mandipropamid, mefenoxam, penconazole, propiconazole, pyroquilon, sedaxane or thiabendazole.
